(19) United States
(12) Patent Application Publication
(10) Pub. No.: US 2006/0035977 A1
(43) Pub. Date: Feb. 16, 2006

(54) Uses of acylated aminopropanediols and sulphur and nitrogen analogues of same f

(75) Inventor: Jamila Najib, Santes (FR)

Correspondence Address: NIXON & VANDERHYE, PC, 901 NORTH GLEBE ROAD, 11TH FLOOR, ARLINGTON, VA, 22203, US

(73) Assignee: Gentif, Loos (FR)

(21) Appl. No.: 10/542,056

(22) PCT Filed: Feb. 12, 2004
(86) PCT No.: PCT/FR04/00320

§ 371 Date: Sep. 21, 2005

(30) Foreign Application Priority Data
Feb. 12, 2003 (FR) 03/01689

(51) Int. Cl. A61K 31/16

(52) U.S. Cl. 514/599

(57) ABSTRACT

The invention relates to the use of molecules, particularly in the fields of human and veterinary health and cosmetics. The inventive compounds are acylated aminopropanediols and the nitrogen- and sulfur-containing analogues thereof and have advantageous pharmacological and cosmetic properties. In particular, the inventive compounds can be used to prevent and/or treat dyslipidemias, cardiovascular diseases, syndrome X, restenosis, diabetes, obesity, hypertension, some cancers, dermatological diseases, and, in the field of cosmetics, to combat skin ageing and the effects of same, in particular the development of wrinkles and the like.

The invention relates to the use of acylated aminopropanediol derivatives and the nitrogen- and sulfur-containing analogues thereof, pharmaceutical and cosmetic compositions comprising same, the therapeutic applications thereof, in particular to prevent or treat cardiovascular diseases, syndrome X, restenosis, diabetes, obesity, hypertension, some cancers, dermatological diseases and in the field of cosmetics; to prevent or treat the effects of skin ageing, in particularly the development of wrinkles and the like.

In particular, the inventive compounds have advantageous antioxidant and anti-inflammatory pharmacological properties. The invention also describes methods of therapeutic treatment using said compounds and the pharmaceutical and cosmetic compositions comprising same.

Atherosclerosis and the cardiovascular complications thereof are the leading cause of morbidity and mortality in highly industrialized countries. Atherosclerosis and its complications are also an important consequence of type II diabetes. A clear cause-effect relationship has been demonstrated between dyslipidemias and cardiovascular diseases. Elevated levels of circulating LDL-cholesterol are unfavorable. The risk associated with high LDL-cholesterol is amplified by elevated triglyceride levels. The importance of the stability of atherosclerotic lesions in the occurrence of cardiovascular accidents has also been demonstrated. The role of LDL oxidation in the development of atherosclerotic plaque and weakening thereof is better understood.

Pharmacological treatments of atherosclerosis are aimed at lowering circulating levels of cholesterol and triglycerides, increasing the stability of atherosclerotic plaque, decreasing mechanical constraints on the vessels (lowering blood pressure) and reducing accessory risk factors such as diabetes.

Fibrates and statins are among the medicaments currently used in the treatment of dyslipidemias. Metformin, sulfonylurea, thiazolidinediones are used in the treatment of type II diabetes.

Fibrates are widely used in the treatment of hypertriglyceridemias. They also have beneficial effects on hypercholesterolemia. Generally they are well tolerated but may cause side effects such as cutaneous reactions, neurological effects, muscle and gastrointestinal effects. Toxicities are rare (renal, muscle, joint, skin, hepatitis, etc.). Their carcinogenic potential is high in rodents but this has not been demonstrated in man.

Statins are widely used in the treatment of hypercholesterolemia. It has been shown that treating patients who have had a first vascular accident considerably reduces the risk of recurrence. Signs or symptoms of hepatitis or myopathy have been described occasionally.

Thiazolidinediones (troglitazone) have recently come into use for the treatment of insulin resistance. For this reason, post-marketing experience is insufficient to make an objective estimate of the full adverse effect profile of these drugs. In this context, the observed increase in the frequency of colon tumors in an animal model predisposed to colon cancer (Min mice with an APC gene mutation) is unfavorable. Moreover, one thiazolidinedione (troglitazone) was very recently withdrawn from the market due to problems with hepatic toxicity.

The principal drugs used for the pharmacological treatment of atherosclerosis (fibrates, statins) have a pleiotropic spectrum of action. Fibrates activate a class of nuclear receptors (PPARα, PPARγ, etc.) involved in coordinating the expression of proteins responsible for lipid transport or metabolism. The pleiotropic nature of the fibrate spectrum of action lies in the diversity of PPAR target genes. Statins reduce de novo cholesterol synthesis by inhibiting the activity of HMG-COA reductase.

The present invention relates to the use as medicament of a family of compounds exhibiting advantageous pharmacological properties and which can be used for the preventive or curative treatment of various pathologies.

The compounds of the invention are represented by general formula (I): 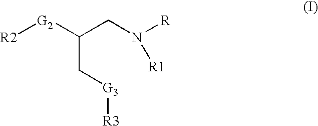
in which:

G2 and G3 independently represent an oxygen atom, a sulfur atom or a N—R4 group, G2 and G3 not simultaneously representing a N—R4 group,

R and R4 independently represent a hydrogen atom or a linear or branched alkyl group, saturated or not, optionally substituted, containing from 1 to 5 carbon atoms,

R1, R2 and R3, which are the same or different, represent a hydrogen atom, a CO—R5 group or a group corresponding to the formula CO—(CH₂)₂ₙ₊₁—X—R6, at least one of the groups R1, R2 or R3 being a group corresponding to the formula CO—(CH₂)₂ₙ₊₁—X—R6,

R5 is a linear or branched alkyl group, saturated or not, optionally substituted, possibly comprising a cyclic group, the main chain of which contains from 1 to 25 carbon atoms,

X is a sulfur atom, a selenium atom, a SO group or a SO₂ group,

n is a whole number comprised between 0 and 11,

R6 is a linear or branched alkyl group, saturated or not, optionally substituted, possibly comprising a cyclic group, the main chain of which contains from 3 to 23 carbon atoms, preferably 10 to 23 carbon atoms and optionally one or more heterogroups, selected in the group consisting of an oxygen atom, a sulfur atom, a selenium atom, a SO group and SO₂ group.

In compounds represented by general formula (I) according to the invention, the R5 group or groups, which are the same or different, preferably represent a linear or branched alkyl group, saturated or unsaturated, substituted or not, the main chain of which contains from 1 to 20 carbon atoms, even more preferably 7 to 17 carbon atoms, still more preferably 14 to 17. In compounds represented by general formula (I) according to the invention, the R5 group or groups, which are the same or different, can also represent a lower alkyl group containing 1 to 6 carbon atoms, such as in particular the methyl, ethyl, propyl, isopropyl, butyl, isobutyl, tertbutyl, pentyl or hexyl group.

In compounds represented by general formula (I) according to the invention, the R6 group or groups, which are the same or different, preferably represent a linear or branched alkyl group, saturated or unsaturated, substituted or not, the main chain of which contains from 3 to 23 carbon atoms, preferably from 13 to 20 carbon atoms, even more preferably from 14 to 17 carbon atoms, and still more preferably 14 carbon atoms.

Specific examples of saturated long chain alkyl groups for R5 or R6 are in particular the groups C₇H₁₅, C₁₀H₂₁, C₁₁H₂₃, C₁₃H₂₇, C₁₄H₂₉, C₁₅H₃₁, C₁₆H₃₃, C₁₇H₃₅. Specific examples of unsaturated long chain alkyl groups for R5 or R6 are in particular the groups C₁₄H₂₇, C₁₄H₂₅, C₁₅H₂₉, C₁₇H₂₉, C₁₇H₃₁, C₁₇H₃₃, C₁₉H₂₉, C₁₉H₃₁, C₂₁H₃₁, C₂₁H₃₅, C₂₁H₃₇, C₂₁H₃₉, C₂₃H₄₅ or the alkyl chains of eicosapentanoic (EPA) C_{20:5} (5, 8, 11, 14, 17) and docosahexanoic (DHA) C_{22:6} (4, 7, 10, 13, 16, 19) acids.

Examples of branched long chain alkyl groups are in particular the groups (CH₂)_{n′}—CH(CH₃)C₂H₅, (CH═C(CH₃)—(CH₂)₂)_{n′}—CH═C(CH₃)₂ ou (CH₂)₂ₓ₊₁—C(CH₃)₂—(CH₂)_{n′″—CH}₃ (x being a whole number equal to or comprised between 1 and 11, n′ being a whole number equal to or comprised between 1 and 22, n″ being a whole number equal to or comprised between 1 and 5, n′″ being a whole number equal to or comprised between 0 and 22, and (2x+n′″) being less than or equal to 22, preferably less than or equal to 20).

As indicated earlier, the alkyl groups R5 or R6 can optionally comprise a cyclic group. Examples of cyclic groups are in particular cyclopropyl, cyclobutyl, cyclopentyl and cyclohexyl.

As indicated earlier, the alkyl groups R5 or R6 can optionally be substituted by one or more substituents, which are the same or different. The substituents are preferably selected in the group consisting of a halogen atom (iodine, chlorine, fluorine, bromine) and a —OH, ═O, —NO₂, —NH₂, —CN, —O—CH₃, —CH₂—OH, —CH₂OCH₃, —CF₃ and —COOZ group (Z being a hydrogen atom or an alkyl group, preferably containing from 1 to 5 carbon atoms).

The invention also has as object the use of the optical and geometrical isomers of said compounds, the racemates, salts, hydrates thereof and the mixtures thereof.

In a preferred manner, the invention concerns the use of compounds represented by formula (I) in which the groups G2R2 and G3R3 do not simultaneously represent hydroxyl groups.

Compounds represented by formula (Ia) are compounds corresponding to formula (I) according to the invention in which a single one of the groups R1, R2 or R3 represents a hydrogen atom.

Compounds represented by formula (Ib) are compounds corresponding to formula (I) according to the invention in which two of the groups R1, R2 or R3 represent a hydrogen atom.

The invention also has as object the use of prodrugs of compounds represented by formula (I) or of compositions comprising same, which, after administration to a subject, are converted to compounds represented by formula (I) which display therapeutic activities similar to compounds represented by formula (I).

Moreover, in the group CO—(CH₂)₂ₙ₊₁—X—R6, X most preferably represents a sulfur or selenium atom and advantageously a sulfur atom.

Moreover, in the group CO—(CH₂)₂ₙ₊₁—X—R6, n is preferably comprised between 0 and 3, more specifically comprised between 0 and 2 and in particular is equal to 0.

In the compounds represented by general formula (I) according to the invention, R6 can contain one or more heterogroups, preferably 0, 1 or 2, more preferably 0 or 1, selected in the group consisting of an oxygen atom, a sulfur atom, a selenium atom, a SO group or a SO₂ group.

A specific example of a CO—(CH₂)₂ₙ₊₁—X—R6 group according to the invention is the group CO—CH₂—S—C₁₄H₂₉.

Preferred compounds in the spirit of the invention are therefore compounds represented by general formula (I) hereinabove in which at least one of the groups R1, R2 and R3 represents a CO—(CH₂)₂ₙ₊₁—X—R6 group in which X represents a sulfur or selenium atom and preferably a sulfur atom and/or R6 is a saturated and linear alkyl group containing from 3 to 23 carbon atoms, preferably 13 to 20 carbon atoms, preferably 14 to 17, more preferably 14 to 16, and even more preferably 14 carbon atoms.

Other particular inventive compounds are those in which at least two of the groups R1, R2 and R3 are CO—(CH₂)₂ₙ₊₁—X—R6 groups, which are the same or different, in which X represents a sulfur or selenium atom and preferably a sulfur atom.

Particular compounds according to the invention are those in which G2 represents an oxygen or sulfur atom, and preferably an oxygen atom. In said compounds, R2 advantageously represents a group corresponding to the formula CO—(CH₂)₂ₙ₊₁—X—R6 such as defined hereinabove.

Particularly preferred compounds are compounds represented by general formula (I) hereinabove in which:
G3 is a N—R4 group in which R4 is a hydrogen atom or a methyl group, and G2 is an oxygen atom; and/or
R2 represents a CO—(CH₂)₂ₙ₊₁—X—R6 group such as defined hereinabove.

Other preferred compounds are compounds represented by general formula (I) hereinabove in which R1, R2 and R3, which are the same or different, preferably the same, represent a CO—(CH₂)₂ₙ₊₁—X—R6 group such as defined hereinabove, in which X represents a sulfur or selenium atom and preferably a sulfur atom and/or R6 is a saturated and linear alkyl group containing from 13 to 17 carbon atoms, preferably 14 to 17, even more preferably 14 carbon atoms, in which n is preferably comprised between 0 and 3, and in particular is equal to 0. More specifically, preferred compounds are compounds represented by general formula (I) in which R1, R2 and R3 represent CO—CH₂—S—C₁₄H₂₉ groups.

Examples of preferred inventive compounds are given in FIG. 1.

Thus, the invention more particularly has as object the use of compounds represented by formula (I) selected in the group consisting of:

3-(tetradecylthioacetylamino)propane-1,2-diol;

1-tetradecylthioacetylamino-2,3-(dipalmitoyloxy)propane;

3-tetradecylthioacetylamino-1,2-(ditetradecylthioacetyloxy)propane;

3-palmitoylamino-1,2-(ditetradecylthioacetyloxy)propane;

1,3-di(tetradecylthioacetylamino)propan-2-ol;

1,3-diamino-2-(tetradecylthioacetyloxy)propane;

1,3-ditetradecylthioacetylamino-2-(tetradecylthioacetyloxy)propane;

1,3-dioleylamino-2-(tetradecylthioacetyloxy)propane;

1,3-ditetradecylthioacetylamino-2-(tetradecylthioacetylthio)propan; and

1-tetradecylthioacetylamino-2,3-di(tetradecylthioacetylthio)propane.

Thus, the invention has as object the use of at least one compound such as described hereinabove for preparing pharmaceutical compositions intended for the treatment of various pathologies, particularly pathologies involving a deregulation of lipid and/or glucose metabolism, pathologies related to inflammation, and/or pathologies related to cell proliferation and/or differentiation.

The pathologies related to deregulations of lipid and/or glucose metabolism which are treated in accordance with the invention are selected in particular in the group consisting of metabolic syndrome (syndrome X), diabetes, atherosclerosis and obesity.

The pathologies related to inflammation which are treated in accordance with the invention are selected in particular in the group consisting of atherosclerosis, an allergy, asthma, eczema, psoriasis and pruritus.

The pathologies related to cell proliferation and/or differentiation which are treated in accordance with the invention are selected in particular in the group consisting of carcinogenesis, psoriasis and atherosclerosis.

More particularly, the invention has as object the use of compounds represented by formula (I) such as defined hereinabove for preparing a pharmaceutical composition for the treatment or prophylaxis of cardiovascular diseases, syndrome X, restenosis, type I or II diabetes, preferably type II, obesity, hypertension, in particular arterial hypertension, cancers, in particular cancer of the anus, rectum, colon, intestine, duodenum, stomach, prostate, testicles, bladder, kidney, pancreas, liver, larynx, breast, lungs, leukemia and melanomas, and dermatological diseases. The invention also relates to the use thereof in cosmetic compositions or for preparing cosmetic compositions, in order to prevent or treat the effects of intrinsic or extrinsic (due to the sun's rays in particular) skin ageing, characterized in particular by the development of wrinkles, spots on the skin and the like. In fact, it was found in a surprising manner that compounds represented by formula (I) concurrently exhibit PPAR activator, antioxidant and anti-inflammatory properties.

The invention also has as object a pharmaceutical or cosmetic composition comprising, in a pharmaceutically or cosmetically acceptable support, a compound represented by general formula (I) such as described hereinabove, optionally in association with another active therapeutic agent, intended for the preventive or curative treatment of the pathologies and disorders noted hereinabove.

The invention also has as object a method for treating the pathologies and disorders noted hereinabove, comprising administering to a subject, particularly animal or in particular human, an effective dose of a compound represented by formula (I) or of a pharmaceutical composition such as defined hereinabove. Treatment is understood to mean either preventive or curative treatment.

Advantageously, the compounds represented by formula (I) which are used are such as defined hereinabove.

The pharmaceutical or cosmetic compositions according to the invention advantageously comprise one or more pharmaceutically or cosmetically acceptible excipients or vehicles. Examples include pharmaceutically or cosmetically compatible saline, physiologic, isotonic, buffered solutions and the like, known to those skilled in the art. The compositions may contain one or more agents or vehicles selected from among dispersives, solubilizers, stabilizers, surfactants, preservatives, and the like. Agents or vehicles that may be used in the formulations (liquid and/or injectable and/or solid) comprise in particular methylcellulose, hydroxymethylcellulose, carboxymethylcellulose, polysorbate 80, mannitol, gelatin, lactose, vegetable oils, acacia and the like. The compositions may be formulated as injectable suspensions, gels, oils, tablets, suppositories, powders, gelatin capsules, capsules, and the like, possibly by means of pharmaceutical forms or devices allowing sustained and/or delayed release. For this type of formulation, an agent such as cellulose, carbonates or starches is advantageously used.

The compounds or compositions of the invention may be administered in different ways and in different forms. For instance, they may be administered systemically, by the oral route, parentally, by inhalation or by injection, such as for example by the intravenous, intramuscular, subcutaneous, transdermal, intra-arterial route, etc. For injections, the compounds are generally prepared in the form of liquid suspensions, which may be injected through syringes or by infusion, for instance. In this respect, the compounds are generally dissolved in pharmaceutically compatible saline, physiologic, isotonic, buffered solutions and the like, known to those skilled in the art. For instance, the compositions may contain one or more agents or vehicles selected from among dispersives, solubilizers, emulsifiers, stabilizers, surfactants, preservatives, buffers, and the like. Agents or vehicles that may be used in the liquid and/or injectable formulations comprise in particular methylcellulose, hydroxymethylcellulose, carboxymethylcellulose, polysorbate 80, mannitol, gelatin, lactose, vegetable oils, acacia, liposomes, and the like.

The compositions may thus be administered in the form of gels, oils, tablets, suppositories, powders, gelatin capsules, capsules, aerosols, and the like, possibly by means of pharmaceutical forms or devices allowing sustained and/or delayed release. For this type of formulation, an agent such as cellulose, carbonates or starches is advantageously used.

The compounds may be administered orally in which case the agents or vehicles used are preferably selected in the group consisting of water, gelatin, gums, lactose, starch, magnesium stearate, talc, an oil, polyalkylene glycol, and the like.

For parenteral administration, the compounds are preferably administered in the form of solutions, suspensions or emulsions in particular with water, oil or polyalkylene glycols to which, in addition to preservatives, stabilizers, emulsifiers, etc., it is also possible to add salts to adjust osmotic pressure, buffers, and the like.

For a cosmetic use, the inventive compounds may be administered in any common cosmetic formulation, in particular cream, such as for example care creams, sun creams, oils, gels, lotions and the like.

It is understood that the injection rate and/or injected dose may be adapted by those skilled in the art according to the patient, the pathology, the mode of administration, etc. Typically, the compounds are administered at doses ranging from 1 μg to 2 g per dose, preferably from 0.1 mg to 1 g per dose. The doses may be administered once a day or several times a day, as the case may be. Moreover, the compositions of the invention may further comprise other active substances or agents.

The invention also concerns methods for preparing the hereinabove compounds. The compounds of the invention can be prepared from commercially available products, by employing a combination of chemical reactions known to those skilled in the art.

According to one method of the invention, compounds represented by formula (I) in which (i) G2 and G3 are oxygen or sulfur atoms or a N—R4 group, (ii) R and, as the case may be, R4, represent an identical linear or branched alkyl group, saturated or not, optionally substituted, containing from 1 to 5 carbon atoms and (iii) R1, R2 and R3, which are the same or different, represent a CO—R5 group or a CO—(CH₂)₂ₙ₊₁—X—R6 group, are obtained from a compound represented by formula (I) in which (i) G2 or G3 are oxygen or sulfur atoms or a NH group, (ii) R is a hydrogen atom and (iii) R1, R2 and R3, which are the same or different, represent a CO—R5 or CO—(CH₂)₂ₙ₊₁—X—R6 group, and a compound corresponding to the formula A1-LG in which A1 represents the group R or, as the case may be, R4 and LG is a reactive group selected for example in the group consisting of Cl, Br, mesyl, tosyl, etc., possibly in the presence of coupling agents or activators known to those skilled in the art.

In a first embodiment, compounds represented by formula (I) in which (i) G2 and G3 are oxygen or sulfur atoms or a NH group, (ii) R is a hydrogen atom and (iii) R1, R2 and R3, which are the same, represent a CO—(CH₂)₂ₙ₊₁—X—R6 group, are obtained from a compound represented by formula (I) in which (i) G2 or G3 are oxygen or sulfur atoms or a NH group, (ii) R is a hydrogen atom and (iii) R1, R2 and R3 are hydrogen atoms and a compound corresponding to the formula A°—CO-A in which A is a reactive group selected for example in the group consisting of OH, Cl, O—CO-A° and O—R7, R7 being an alkyl group, and A° is the (CH₂)₂ₙ₊₁—X—R6 group, possibly in the presence of coupling agents or activators known to those skilled in the art.

Compounds represented by formula (I) according to the invention in which (i) G2 and G3 are oxygen atoms or a NH group, (ii) R is a hydrogen atom and (iii) R1, R2 and R3 are hydrogen atoms or represent a CO—R5 or CO—(CH₂)₂ₙ₊₁—X—R6 group can be obtained by different methods which enable the synthesis of compounds in which the groups carried on a same heteroatom (nitrogen or oxygen) have the same meaning.

According to a first embodiment, a molecule of 1-aminoglycerol, 1,3-diaminoglycerol or 1,2-diaminoglycerol (obtained by adapting the protocol described by (Morris, Atassi et al. 1997)) is reacted with a compound corresponding to the formula A°—CO-A1 in which A1 is a reactive group selected for example in the group consisting of OH, Cl and OR7, R7 being an alkyl group, and A° is the R5 group or the (CH₂)₂ₙ₊₁—X—R6 group, possibly in the presence of coupling agents or activators known to those skilled in the art. Said reaction respectively yields particular forms of compounds represented by formula (I), named compounds (IIa-c), and can be carried out by adapting the protocols described by (Urakami and Kakeda 1953); (Shealy, Frye et al. 1984); (Marx, Piantadosi et al. 1988); (Rahman, Ziering et al. 1988) and (Nazih, Cordier et al. 1999). In compounds (IIb-c), the groups carried on a same heteroatom, respectively, (R1 and R3) and (R1 and R2) have the same meaning.

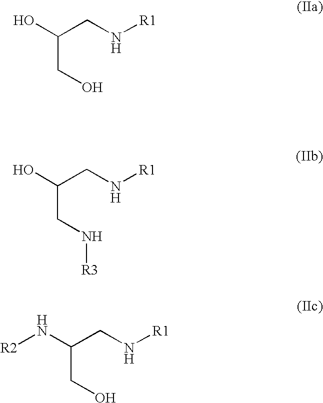

Compounds represented by formula (I) according to the invention in which (i) G2 and G3 are oxygen atoms or a NH group, (ii) R is a hydrogen atom and (iii) R1, R2 and R3, which are the same or different, represent a CO—R5 or CO—(CH₂)₂ₙ₊₁—X—R6 group, can be obtained from a compound having formula (IIa-c) and a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art. Said reaction enables the synthesis of compounds in which the groups carried on a same heteroatom (nitrogen or oxygen), respectively (R1 and R2), (R1 and R3) or (R2 and R3) have the same meaning. Advantageously, said reaction is carried out according to the protocol described for example in (Urakami and Kakeda 1953) and (Nazih, Cordier et al. 1999).

According to another particular method of the invention (diagram 1), compounds represented by formula (I) in which (i) G2 and G3 are oxygen atoms or a NH group (ii) R is a hydrogen atom and (iii) R1, R2 and R3, which are the same or different, represent a CO—R5 or CO—(CH₂)₂ₙ₊₁—X—R5 group, can be obtained according to the following steps:
a) reacting 1-aminoglycerol, 1,3-diaminoglycerol or 1,2-diaminoglycerol with a compound (PG)₂O in which PG is a protective group to give a compound having general formula (IIIa-c). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
b) reacting the compound having formula (IIIa-c) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (IVa-c), in which R2 and R3 represent a CO—R5 or CO—(CH₂)₂ₙ₊₁—X—R6 group and PG is a protective group;
c) deprotecting the compound (IVa-c), according to conventional conditions known to those skilled in the art, to give a compound represented by general formula (I) in which (i) G2 and G3 represent an oxygen atom or a NH group, (ii) R and R1 are hydrogen atoms and (iii) R2 and R3 represent a CO—R5 or CO—(CH₂)₂ₙ₊₁—X—R6 group;
d) reacting a compound represented by general formula (I) in which (i) G2 and G3 represent an oxygen atom or a NH group, (ii) R and R1 are hydrogen atoms and (iii) R2 and R3 represent a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly-in the presence of coupling agents or activators known to those skilled in the art.

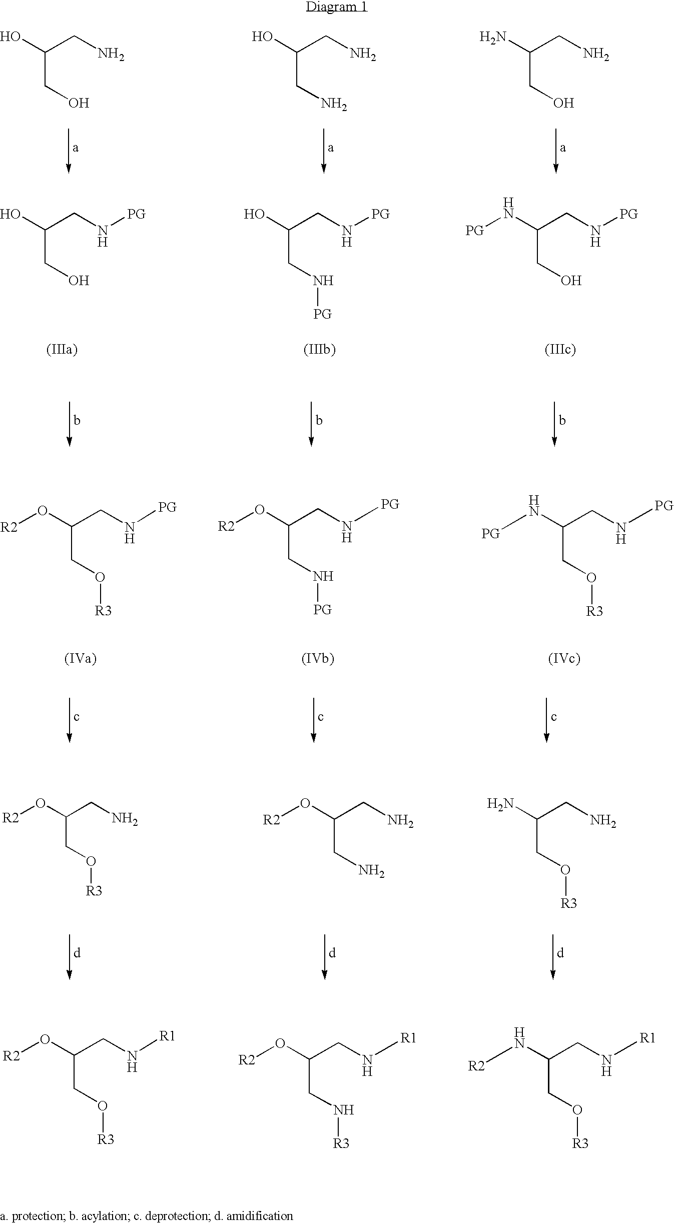

Compounds represented by formula (I) according to the invention in which (i) G2 and G3 are oxygen atoms, (ii) R is a hydrogen atom and (iii) R1, R2 and R3, which are the same or different, represent a CO—R5 or CO—(CH₂)₂ₙ₊₁—X—R6 group, can be obtained in different ways.

According to a first method, a compound represented by formula (I) according to the invention, in which (i) G2 and G3 are oxygen atoms, (ii) R and R2 are hydrogen atoms and (iii) R1, R3, which are the same or different, represent a CO—R5 or CO—(CH₂)₂ₙ₊₁—X—R6 group, is reacted with a compound corresponding to the formula A°—CO-A2 in which A2 a reactive group selected for example in the group consisting of OH and Cl, and A° is the R5 group or the (CH₂)₂ₙ₊₁—X—R6 group, possibly in the presence of coupling agents or activators known to those skilled in the art.

According to this method of preparation, compounds represented by formula (I) in which (i) G2 and G3 are oxygen atoms, (ii) R and R2 are hydrogen atoms and (iii) R1 and R3, which are the same or different, represent a CO—R5 or CO—(CH₂)₂ₙ₊₁—X—R6 group, can be obtained from a compound represented by formula (IIa) such as defined hereinabove and a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

According to another particular inventive method, compounds represented by formula (I) in which (i) G2 and G3 are oxygen atoms, (ii) R is a hydrogen atom and (iii) R1, R2 and R3, which are the same or different, represent a CO—R5 or CO—(CH₂)₂ₙ₊₁—X—R6 group, can be obtained from a compound represented by formula (I) according to the invention in which (i) G2 and G3 are oxygen atoms, (ii) R, R2 and R3 represent a hydrogen atom and (iii) R1 is a CO—R5 or CO—(CH₂)₂ₙ₊₁—X—R6 group (compound of formula (IIa)) according to the following steps (diagram 2):
a) reacting a compound represented by formula (IIa) with a compound PG-E in which PG is a protective group and E is a reactive group selected for example in the group consisting of OH and a halogen, to give a compound represented by general formula (V) in which R1 is a CO—R5 or CO—(CH₂)₂ₙ₊₁—X—R6 group. Advantageously, the reaction can be carried out by adapting the protocols described by (Marx, Piantadosi et al. 1988) and (Gaffney and Reese 1997) in which PG-E can represent triphenylmethyl chloride or 9-phenylxanthene-9-ol or else 9—Chloro-9-phenylxanthene;
b) reacting a compound represented by formula (V) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence: of coupling agents or activators known to those skilled in the art to give a compound represented by general formula (VI), in which R1 and R2, which are the same or different, represent a CO—R5 or CO—(CH₂)₂ₙ₊₁—X—R6 group and PG is a protective group;
c) deprotecting the compound (VI), in conditions known to those skilled in the art, to give a compound represented by general formula (I) in which (i) G2 and G3 are oxygen atoms, (ii) R and R3 are hydrogen atoms and (iii) R1 and R2, which are the same or different, represent a CO—R5 or CO—(CH₂)₂ₙ₊₁—X—R6 group;
d) reacting a compound represented by general formula (I) in which (i) G2 and G3 are oxygen atoms, (ii) R and R3 are hydrogen atoms and (iii) R1 and R2, which are the same or different, represent a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

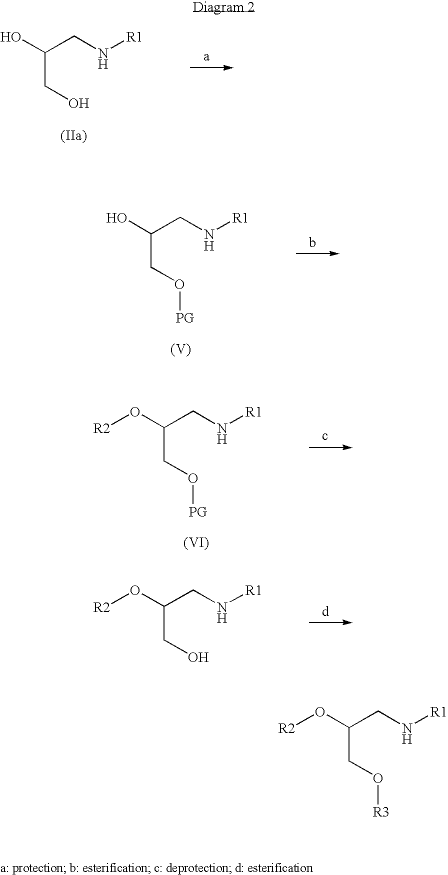

In an advantageous manner, the hereinabove steps are carried out according to the protocols described by (Marx, Piantadosi et al. 1988).

According to another method of the invention, compounds represented by formula (I) in which (i) G2 or G3 represent an oxygen atom or a N—R4 group, (ii) at least one of the groups G2 or G3 represents a N—R4 group, (iii) R and R4 independently represent linear or branched alkyl groups, saturated or not, optionally substituted, containing from 1 to 5 carbon atoms and (iv) R1, R2 and R3, which are the same or different, represent a CO—R5 group or a CO—(CH2)₂ₙ₊₁—X—R6 group, are obtained by reacting a compound represented by formula (I) in which (i) one of the groups G2R2 or G3R3 represents a hydroxyl group and the other group G2R2 or G3R3 represents a NR4R2 or NR4R3 group, respectively, with R2 or R3 representing a CO—R5 group or a CO—(CH₂)₂ₙ₊₁—X—R6 group, (ii) R and R4 independently represent a linear or branched alkyl group, saturated ornot, optionally substituted, containing from 1 to 5 carbon atoms and (iii) R1 represents a CO—R5 group or a CO—(CH2)₂ₙ₊₁—X—R6 group, with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

Compounds represented by formula (I) according to the invention in which (i) one of the groups G2R2 or G3R3 represents a hydroxyl group and the other group G2R2 or G3R3 represents a NR4R2 or NR4R3 group, respectively, with R2 or R3 representing a CO—R5 group or a CO—(CH₂)₂ₙ₊₁—X—R6 group, (ii) R and R4 independently represent linear or branched alkyl groups, saturated or not, optionally substituted, containing from 1 to 5 carbon atoms and (iii) R1 represents a CO—R5 group or a CO—(CH₂)₂ₙ₊₁—X—R6 group, are obtained from compounds represented by formula (I) according to the invention in which one of the groups G2R2 or G3R3 represents a hydroxyl group and the other group G2R2 or G3R3 represents a NR4R2 or NR4R3 group, respectively, with R2 or R3 representing a CO—R5 group or a CO—(CH₂)₂ₙ₊₁—X—R6 group, (ii) R and R4 independently represent a group such as defined hereinabove and (iii) R1 is a hydrogen atom and a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

In a first embodiment, compounds represented by formula (I) according to the invention in which (i) G2 is an oxygen atom, (ii) G3 represents a N—R4 group, (iii) R and R4 independently represent different linear or branched alkyl groups, saturated or not, optionally substituted, containing from 1 to 5 carbon atoms, (iv) R1 and R2 are hydrogen atoms and (v) R3 represents a CO—R5 group or a CO—(CH₂)₂ₙ₊₁—X—R6 group are obtained in the following manner (diagram 3):
a) reacting 1-aminoglycerol with a compound corresponding to the formula R—CHO in which R represents a linear or branched alkyl group, saturated or not, optionally substituted, containing from 1 to 5 carbon atoms and CHO is the aldehyde function in the presence of reducing agents known to those skilled in the art to give a compound represented by formula (VII) in which R is a group such as defined hereinabove. Advantageously, said reaction can be carried out by adapting the protocols described by (Antoniadou-Vyzas, Foscolos et al. 1986);
b) reacting a compound represented by formula (VII) with a compound (PG)₂O in which PG is a protective group to give a compound represented by general formula (VIII). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
c) reacting a compound represented by formula (VIII) with a compound corresponding to the formula LG-E in which E represents a halogen and LG is a reactive group selected for example in the group consisting of mesyl, tosyl, etc., to give a compound represented by general formula (IX) by adapting the method described by (Kitchin, Bethell et al. 1994);
d) reacting a compound represented by formula (IX) with a compound corresponding to the formula R4-NH₂ in which R4 represents a linear or branched alkyl group, saturated or not, optionally substituted, containing from 1 to 5 carbon atoms and NH₂ represents the amine function, according to the method described by (Ramalingan, Raju et al. 1995), to give a compound corresonding to formula (X) in which R and R4, optionally different, are such as defined hereinabove;
e) reacting a compound represented by formula (X) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (XI) in which R and R4 represent different linear or branched alkyl groups, saturated or not, optionally substituted, containing from 1 to 5 carbon atoms, R3 represents the R5 group or the (CH₂)₂ₙ₊₁—X—R6 group and PG is a protective group;
f) deprotecting the compound (XI) in conditions known to those skilled in the art.

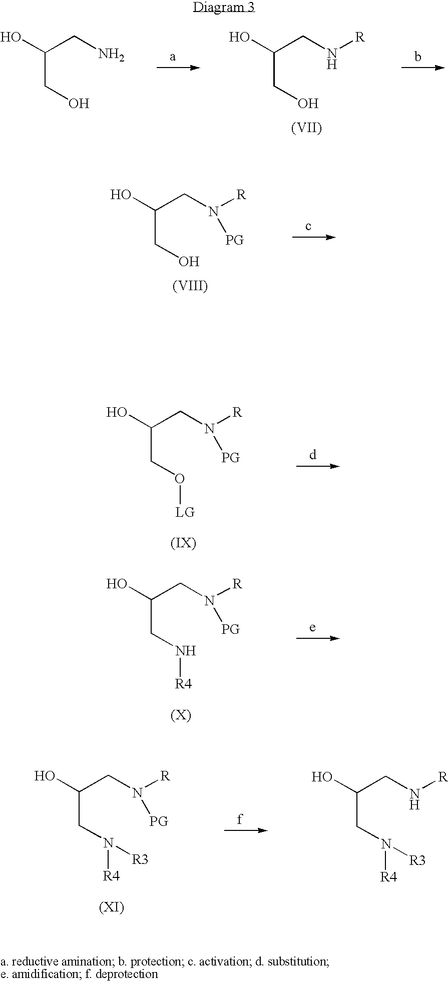

According to a second embodiment, compounds represented by formula (I) according to the invention in which (i) G3 is an oxygen atom, (ii) G2 represents a N—R4 group, (iii) R and R4 represent different linear or branched alkyl groups, saturated or not, optionally substituted, containing from 1 to 5 carbon atoms, (iv) R1 and R3 are hydrogen atoms and (v) R2 represents a CO—R5 group or a CO—(CH₂)₂ₙ₊₁—X—R6 group are obtained in the following manner (diagram 4):
a) reacting a compound represented by formula (VIII) with a compound PG′- E in which PG′ is a protective group and E is a reactive group selected for example in the group consisting of OH or a halogen, to give a compound represented by general formula (XII) in which R represents a linear or branched alkyl group, saturated or not, optionally substituted, containing from 1 to 5 carbon atoms and PG is another protective group such as defined hereinabove. Advantageously, the reaction can be carried out by adapting the protocols described by (Marx, Piantadosi et al. 1988) and (Gaffney and Reese 1997) in which PG′-E can represent triphenylmethyl chloride or 9-phenylxanthene-9-ol or else 9—Chloro-9-phenylxanthene;
b) reacting a compound represented by formula (XII) such as defined hereinabove with a compound corresponding to the formula LG-E in which E represents a halogen and LG is a reactive group selected for example in the group consisting of mesyl, tosyl, etc., to give a compound represented by general formula (XIII) in which R represents a linear or branched alkyl group, saturated or not, optionally substituted, containing from 1 to 5 carbon atoms and PG and PG′ are protective groups, by adapting the method described by (Kitchin, Bethell et al. 1994);
c) reacting a compound represented by formula (XIII) such as defined hereinabove with a compound corresponding to the formula R4-NH₂ in which R4 represents a linear or branched alkyl group, saturated or not, optionally substituted, containing from 1 to 5 carbon atoms and NH₂ represents the amine function, according to the method described by (Ramalingan, Raju et al. 1995), to obtain a compound represented by formula (XIV) in which R and R4 are independently such as defined hereinabove;
d) reacting a compound represented by formula (XIV) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (XV) in which R and R4 independently represent linear or branched alkyl groups, saturated or not, optionally substituted, containing from 1 to 5 carbon atoms, R2 represents a CO—R5 group or a CO—(CH₂)₂ₙ₊₁—X—R6 group, PG and PG′ are protective groups;
e) deprotecting a compound represented by formula (XV) in conventional conditions known to those skilled in the art to obtain a compound represented by general formula (I) according to the invention in which (i) R and R4 independently represent linear or branched alkyl groups, saturated or not, optionally substituted, containing from 1 to 5 carbon atoms, (ii) R1 and R3 are hydrogen atoms and (iii) R2 represents a CO—R5 group or a CO—(CH₂)₂ₙ₊₁—X—R6 group.

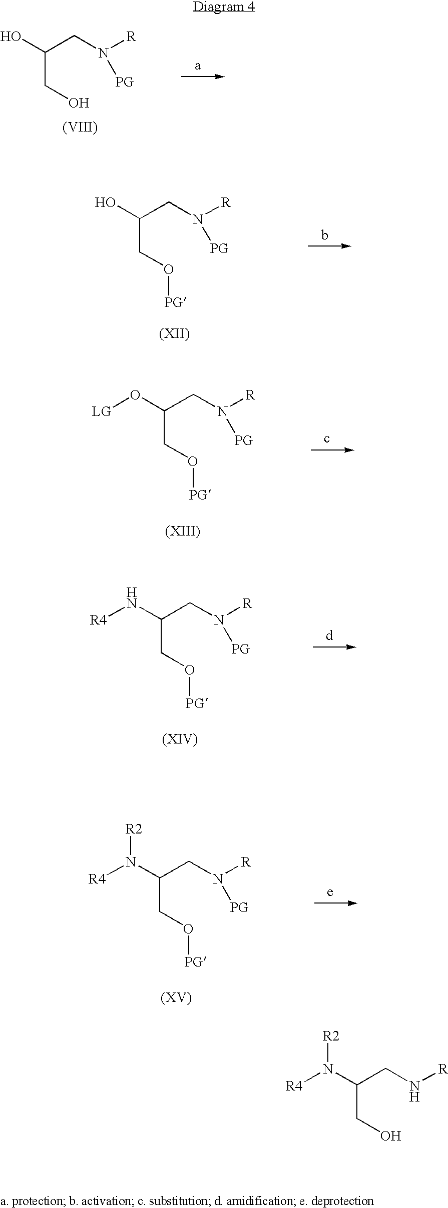

Compounds represented by formula (I) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group can be obtained by different methods.

According to a first embodiment, compounds represented by formula (I) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group, R1, R2 and/or R3 having the same meaning when they are carried on a same heteroatom (sulfur or nitrogen), can be obtained in the following manner (diagram 5A):
a) reacting a compound represented by formula (IIa-c) with a compound corresponding to the formula LG-E in which E represents a halogen and LG is a reactive group selected for example in the group consisting of mesyl, tosyl, etc., to give a compound represented by general formula (XVIa-c);
b) reacting a compound represented by formula (XVIa-c) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XVIIa-c). Advantageously, said reaction can be carried out by adapting the protocol described by (Gronowitz, Herslöf et al. 1978);
c) deprotecting a compound represented by formula (XVIIa-c), in conventional conditions known to those skilled in the art, and for example in basic medium, to give a compound represented by general formula (I) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group;
d) reacting a compound represented by general formula (I) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group, with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

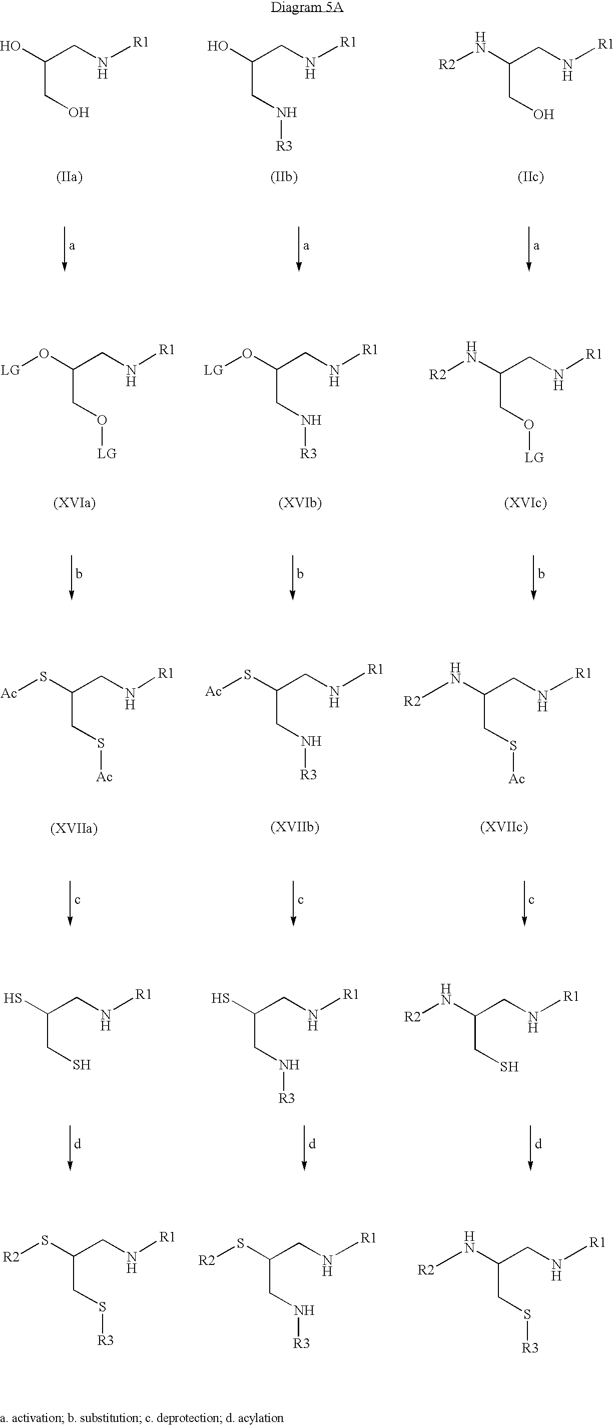

According to a similar synthetic method, compounds having formula (I) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group, R1, R2 and/or R3 having the same meaning when they are carried on a same heteroatom (sulfur or nitrogen), can be prepared in the following manner (diagram 5B):
a) reacting a compound represented by formula (IIa-c) with a compound corresponding to the formula (LG)2 in which LG is a reactive group selected for example in the group consisting of iodine, bromine, etc., possibly in the presence of activators known to those skilled in the art to give a compound represented by general formula (XVId-f);
b) reacting a compound represented by formula (XVId-f) with a compound corresponding to the formula HS⁻B⁺ in which B is a counter-ion selected for example in the group consisting of sodium or potassium, preferably sodium to give a compound represented by general formula (I) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group;
c) reacting a compound represented by general formula (I) in which (i) G2 and G3 represent a sulfur atom or a NH group and (ii) R1, R2 and R3, which are the same or different, represent a hydrogen atom or a CO—R5 or CO—(CH₂)₂ₙ₊₁—X—R6 group, with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

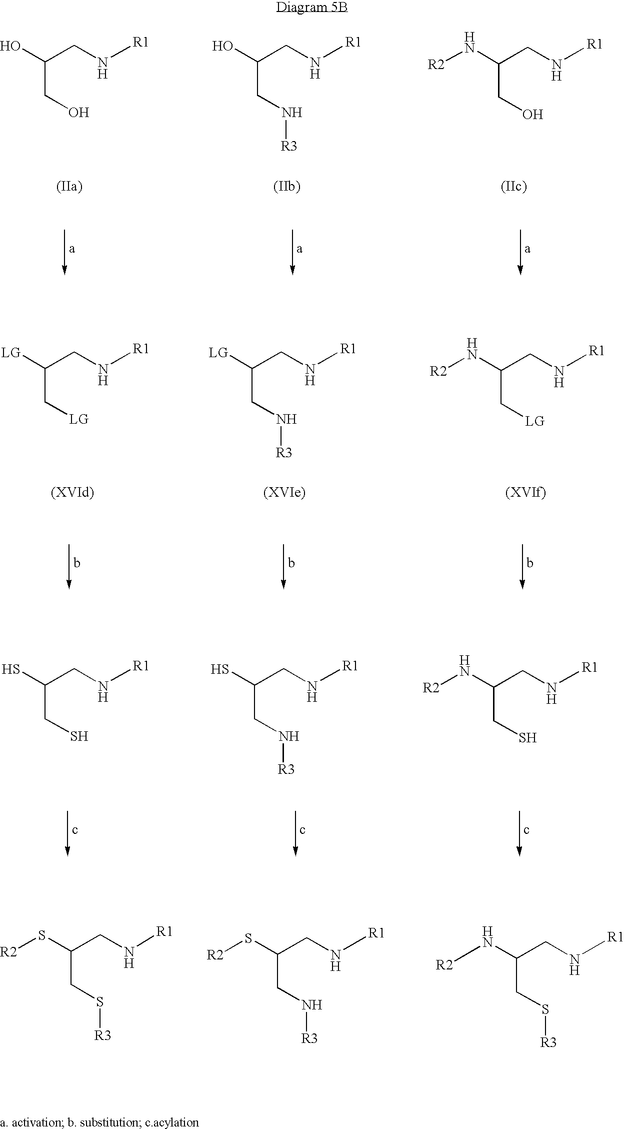

Said reaction enables the synthesis of compounds represented by general formula (I) in which the groups carried on a same heteroatom (nitrogen or sulfur) respectively (R2 and R3), (R1 and R3) and (R1 and R2) have the same meaning.

The above steps can be carried out in an advantageous manner according to the protocols described by (Adams, Doyle et al. 1960) and (Gronowitz, Herslöf et al. 1978).

According to another method of the invention, compounds represented by formula (I) according to the invention in which (i) G2 and G3 are sulfur atoms or a NH group, (ii) R is a hydrogen atom and (iii) R1, R2 and R3 are hydrogen atoms or represent a CO—R5 or CO—(CH₂)₂ₙ₊₁—X—R6 group can be prepared from compounds represented by formula (IIIa-c) by a method comprising (diagram 6):
a) reacting a compound represented by formula (IIIa-c) with a compound corresponding to the formula LG-E in which E represents a halogen and LG is a reactive group selected for example in the group consisting of mesyl, tosyl, etc., to give a compound represented by general formula (XVIIIa-c) in which PG represents a protective group;
b) reacting a compound represented by formula (XVIIIa-c) with a compound corresponding to the formula Ac—S⁻B⁺ in which Ac represents a short acyl group, preferably the acetyl group, and B is a counter-ion selected for example in the group consisting of sodium and potassium, preferably potassium to give a compound represented by general formula (XIXa-c). Advantageously, said reaction can be carried out by adapting the protocol described by (Gronowitz, Herslöf et al. 1978);
c) deprotecting the sulfur atom of a compound (XIXa-c) in conditions known to those skilled in the art, to give a compound represented by general formula (XXa-c);
d) reacting a compound represented by general formula (XXa-c) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXIa-c) in which R2 and R3 represent a CO—R5 or CO—(CH₂)₂ₙ₊₁—X—R6 group;
e) deprotecting a compound represented by formula (XXIa-c) in conventional conditions known to those skilled in the art, to give a compound represented by formula (I) according to the invention in which (i) G2 and G3 are sulfur atoms or a NH group, (ii) R and R1 are hydrogen atoms and (iii) R2 and R3 represent a hydrogen atom, a CO—R5 or CO—(CH₂)₂ₙ₊₁—X—R6 group.
f) reacting a compound represented by formula (I) according to the invention in which (i) G2 and G3 are sulfur atoms or a NH group, (ii) R and R1 are hydrogen atoms and (iii) R2 and R3 represent a hydrogen atom, a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

Said reaction enables the synthesis of compounds represented by general formula (I) in which the groups carried on a same heteroatom (nitrogen or sulfur) respectively (R2 and R3), (R1 and R3) and (R1 and R2) have the same meaning.

Advantageously, the above steps can be carried out according to the protocols described by (Adams, Doyle et al. 1960), (Gronowitz, Herslöf et al. 1978), (Bhatia and Hajdu 1987) and (Murata, Ikoma et al. 1991).

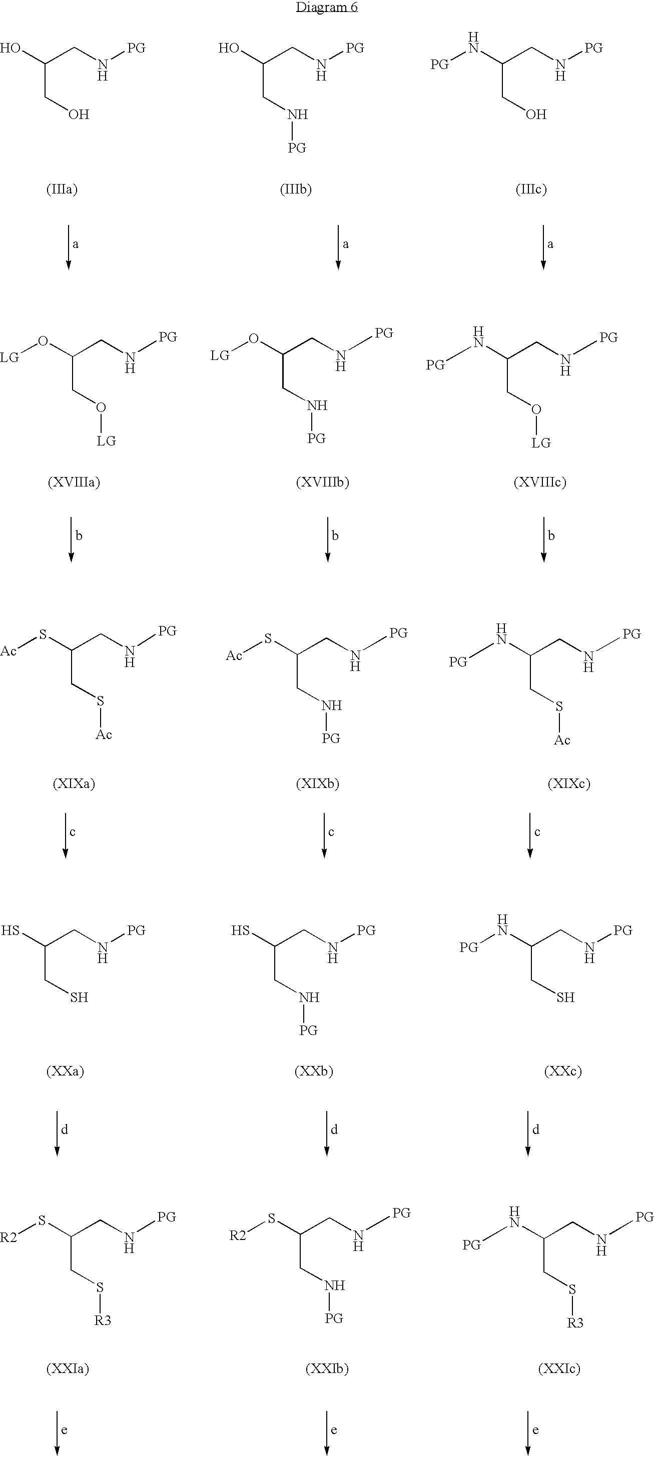

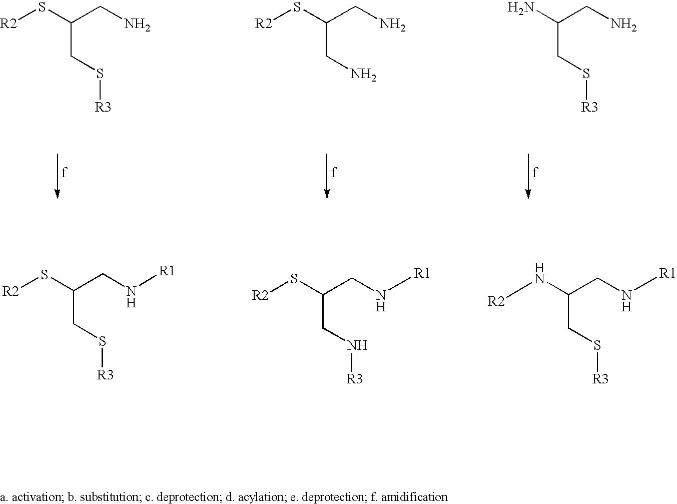

Compounds represented by general formula (I) in which (i) G2 and G3 represent sulfur atoms or a N—R4 group, (ii) R and R4 independently represent a linear or branched alkyl group, saturated or not, optionally substituted, containing from 1 to 5 carbon atoms, (iii) R1, R2 and R3, which are the same or different, represent a CO—R5 group or a CO—(CH₂)₂ₙ₊₁—X—R6 group, are obtained by reacting a compound represented by general formula (I) in which (i) G2 or G3 represent a sulfur atom or a N—R4 group, (ii) R and R4 independently represent groups such as defined hereinabove, (iii) R1 is a hydrogen atom and (iv) R2 and R3, which are the same or different, represent a CO—R5 group or a CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

Compounds represented by general formula (I) in which (i) the groups G2 and G3 represent a sulfur atom or a N—R4 group, (ii) R and R4 independently represent groups such as defined hereinabove, (iii) R1 is a hydrogen atom and (iv) R2 and R3, which are the same or different, represent a CO—R5 group or a CO—(CH₂)₂ₙ₊₁—X—R6 group, can be obtained by the following methods:

In a first embodiment, compounds represented by formula (I) according to the invention in which (i) the group G2 is a sulfur atom, (ii) G3 represents a N—R4 group, (iii) R and R4 independently represent different linear or branched alkyl group, saturated or not, optionally substituted, containing from 1 to 5 carbon atoms, (iv) R1 is a hydrogen atom and (v) R2 and R3, which are the same or different, represent a CO—R5 group or a CO—(CH₂)₂ₙ₊₁—X—R6 group are obtained in the following manner (diagram 7):
a) reacting a compound represented by formula (XI) with a compound corresponding to the formula LG-E in which E represents a halogen and LG is a reactive group selected for example in the group consisting of mesyl, tosyl, etc., to give a compound represented by general formula (XXII) in which PG represents a protective group;
b) reacting a compound represented by formula (XXII) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XXIII). Advantageously, said reaction is carried out by adapting the protocol described by (Gronowitz, Herslöf et al. 1978);
c) deprotecting the sulfur atom of a compound represented by formula (XXIII) in conventional conditions known to those skilled in the art to give a compound represented by general formula (XXIV);
d) reacting a compound represented by general formula (XXIV) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXV) in which R2 and R3, which are the same or different, represent a CO—R5 or CO—(CH₂)₂ₙ₊₁—X—R6 group;
e) deprotectig the compound of formula (XXV) in conditions known to those skilled in the art.

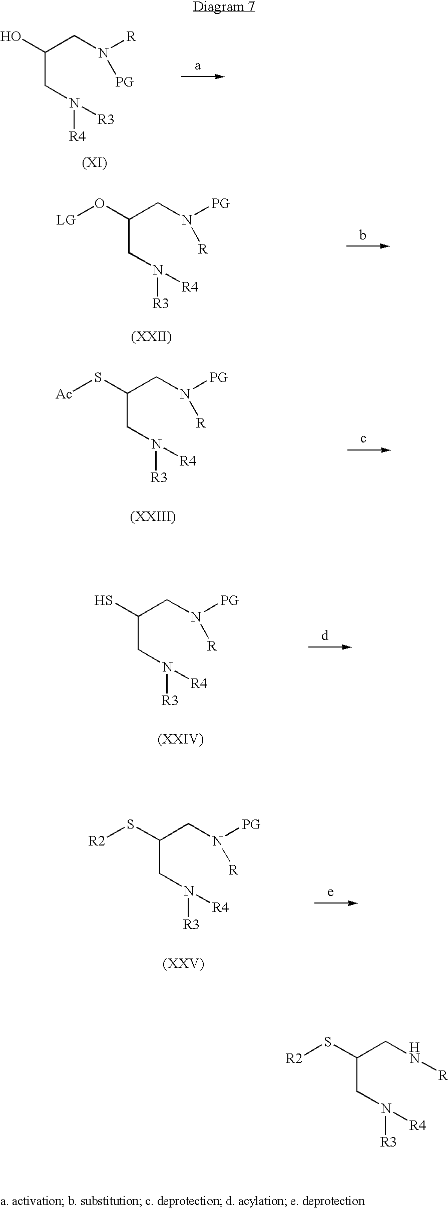

According to another method, compounds represented by formula (I) according to the invention in which (i) G2 represents a N—R4 group, (ii) G3 is a sulfur atom, (iii) R and R4 independently represent different linear or branched alkyl groups, saturated or not, optionally substituted, containing from 1 to 5 carbon atoms, (iv) R1 is a hydrogen atom and (v) R2 and R3, which are the same or different, represent a CO—R5 group or a CO—(CH₁)₂ₙ₊₁—X—R6 group are obtained in the following manner (diagram 8):
a) reacting the compound represented by formula (IX) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XXVI). Advantageously, said reaction can be carried out by adapting the protocol described by (Gronowitz, Herslöf et al. 1978);
b) reacting a compound represented by formula (XXVI) with a compound corresponding to the formula LG-E in which E represents a halogen and LG is a reactive group selected for example in the group consisting of mesyl, tosyl, etc., to give a compound represented by general formula (XXVII) in which PG represents a protective group;
c) reacting the compound (XXVII) with a compound represented by formula R4-NH₂ in which R4 represents a linear or branched alkyl group, saturated or not, optionally substituted, containing from 1 to 5 carbon atoms and NH₂ represents the amine function, according to the method described by (Ramalingan, Raju et al. 1995), to give a compound represented by formula (XXVIII) in which R and R4 independently represent different linear or branched alkyl groups, saturated or not, optionally substituted, containing from 1 to 5 carbon atoms;
d) reacting a compound represented by general formula (XXVIII) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXIX);
e) deprotecting the sulfur atom of a compound represented by formula (XXIX) in conventional conditions known to those skilled in the art to give a compound represented by general formula (XXX);
f) reacting a compound represented by general formula (XXX) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXXI) in which R2 and R3, which are the same or different, represent a CO—R5 or CO—(CH₂)₂ₙ₊₁—X—R6 group;
g) deprotecting a compound represented by formula (XXXI) in conventional conditions known to those skilled in the art.

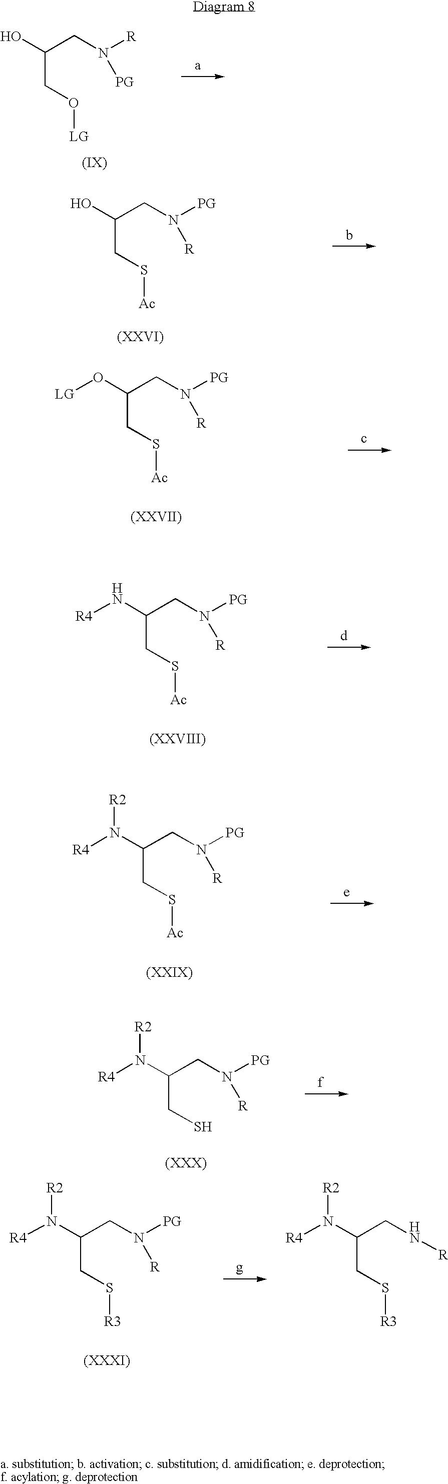

Compounds represented by formula (I) according to the invention in which (i) G2 is a sulfur atom, (ii) G3 is an oxygen atom, (iii) R is a hydrogen atom, (iv) R1 and R2 represent a CO—R5 or CO—(CH₂)₂ₙ₊₁—X—R6 group and (v) R3 is a hydrogen atom or represents a CO—R5 or CO—(CH₂)₂ₙ₊₁—X—R6 group, can be prepared from compounds having formula (V) according to the following method (diagram 9A):
a) reacting the compound (V) with a compound corresponding to the formula LG-E in which E represents a halogen and LG is a reactive group selected for example in the group consisting of mesyl, tosyl, etc., to give a compound represented by general formula (XXXII) in which PG represents a protective group;
b) reacting a compound represented by formula (XXXII) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XXXIII). Advantageously, said reaction can be carried out by adapting the protocol described by (Gronowitz, Herslöf et al. 1978);
c) deprotecting the sulfur atom of a compound (XXXIII), in conventional conditions known to those skilled in the art, to give a compound represented by general formula (XXXIV);
d) reacting a compound represented by general formula (XXXIV) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXXV) in which R1 and R2, which are the same or different, represent a CO—R5 or CO—(CH₂)₂ₙ₊₁—X—R6 group;
e) deprotecting a compound (XXXV) in conventional conditions known to those skilled in the art to give a compound represented by general formula (I) in which G2 is a sulfur atom, G3 is an oxygen atom, R and R3 are hydrogen atoms and R1 and R2, which are the same or different, represent a CO—R5 or CO—(CH₂)₂ₙ₊₁—X—R6 group;
f) reacting a compound represented by general formula (I) in which (i) G2 is a sulfur atom, (ii) G3 is an oxygen atom, (iii) R and R3 are hydrogen atoms and (iv) R1 and R2, which are the same or different, represent a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

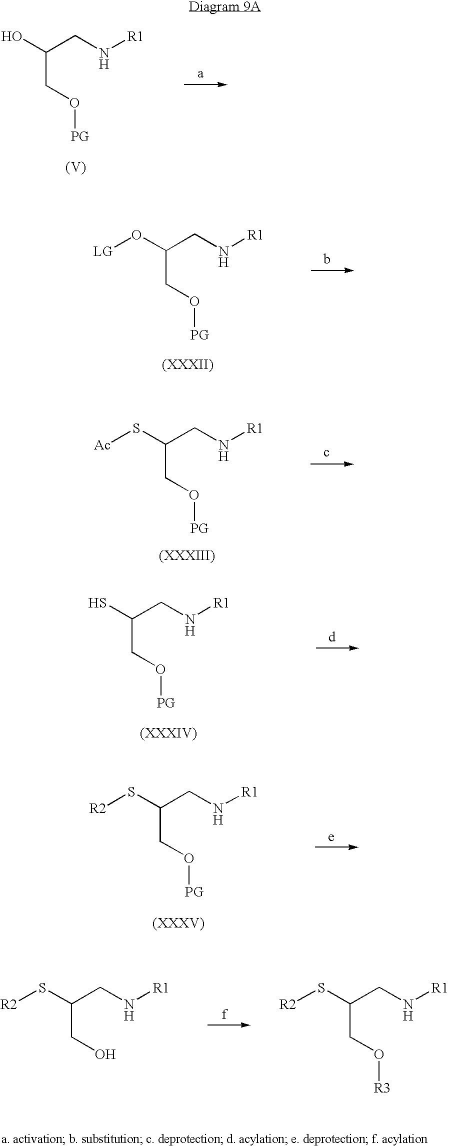

According to a similar method of synthesis, compounds represented by formula (I) according to the invention in which (i) G2 is a sulfur atom, (ii) G3 is an oxygen atom, (iii) R is a hydrogen atom, (iv) R1 and R2 represent a CO—R5 or CO—(CH₂)₂ₙ₊₁—X—R6 group and (v) R3 is a hydrogen atom or represents a CO—R5 or CO—(CH₂)₂ₙ₊₁—X—R6 group, can be prepared from compounds of formula (V) by the following method (diagram 9B):
a) reacting the compound (V) with a compound corresponding to the formula (LG)₂ in which LG is a reactive group selected for example in the group consisting of iodine, bromine, etc., to give a compound represented by general formula (XXXIIa) in which PG represents a protective group;
b) reacting a compound represented by formula (XXXIIa) with a compound corresponding to the formula HS⁻B⁺ in which B is a counter-ion selected for example in the group consisting of sodium and potassium, preferably sodium to give a compound represented by general formula (XXXIV);
c) reacting a compound represented by general formula (XXXIV) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XXXV) in which R1 and R2, which are the same or different, represent a CO—R5 or CO—(CH₂)₂ₙ₊₁—X—R6 group;
d) deprotecting the compound (XXXV) in conventional conditions known to those skilled in the art to give a compound represented by general formula (I) in which G2 is a sulfur atom, G3 is an oxygen atom, R and R3 are hydrogen atoms and R1 and R2, which are the same or different, represent a CO—R5 or CO—(CH₂)₂ₙ₊₁—X—R6 group;
e) reacting a compound represented by general formula (I) in which (i) G2 is a sulfur atom, (ii) G3 is an oxygen atom, (iii) R and R3 are hydrogen atoms and (iv) R1 and R2, which are the same or different, represent a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

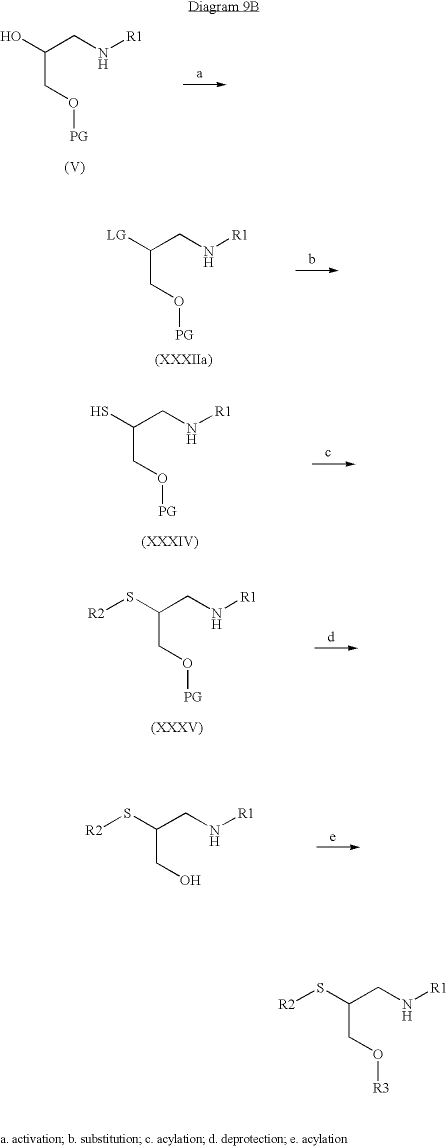

Compounds represented by formula (I) according to the invention in which (i) G2 is a sulfur atom, (ii) G3 is an oxygen atom, (iii) R is a hydrogen atom, (iv) R1 and R3 represent a hydrogen atom or a CO—R5 or CO—(CH₂)₂ₙ₊₁—X—R6 group, which are the same or different, and (v) R2 represents a CO—R5 or CO—(CH₂)₂ₙ₊₁—X—R6 group, can be prepared from compounds having formula (IIIa) by the following method (diagram 10):
a) reacting a compound represented by formula (IIIa) with a compound PG′-E in which PG′ is a protective group and E is a reactive group selected for example in the group consisting of OH and a halogen, to give a compound represented by general formula (XXXVI) in which PG is another protective group such as defined earlier. In an advantageous manner, the reaction can be carried out by adapting the protocols described by (Marx, Piantadosi et al. 1988) and (Gaffney and Reese 1997) in which PG-E can represent triphenylmethyl chloride or 9-phenylxanthene-9-ol or else 9—Chloro-9-phenylxanthene;
b) reacting the compound (XXXVI) with a compound corresponding to the formula LG-E in which E represents a halogen and LG is a reactive group selected for example in the group consisting of mesyl, tosyl, etc., to give a compound represented by general formula (XXXVII) in which PG and PG′ represent judiciously selected protective groups such as defined hereinabove;
c) reacting a compound represented by formula (XXXVII) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (XXXVIII). Advantageously, said reaction can be carried out by adapting the protocol described by (Gronowitz, Herslöf et al. 1978);
d) deprotecting the sulfur atom of a compound (XXXVIII), in conventional conditions known to those skilled in the art, to give a compound represented by general formula (XXXIX);
e) reacting a compound represented by general formula (XXXIX) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XL) in which R2 represents a CO—R5 or CO—(CH₂)₂ₙ₊₁—X—R6 group;
f) deprotecting a compound (XL) in conventional conditions known to those skilled in the art to give a compound represented by general formula (I) in which G2 is a sulfur atom, G3 is an oxygen atom, R, R1 and R3 are hydrogen atoms and R represents a CO—R5 or CO—(CH₂)₂ₙ₊₁—X—R6 group (compound XLI);
g) reacting a compound represented by formula (XLI) with a compound (PG)₂O in which PG is a protective group to give a compound represented by general formula (XLII). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
h) reacting a compound represented by general formula (XLII) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (XLIII);
i) deprotecting a compound (XLIII) in conventional conditions known to those skilled in the art to give a compound represented by general formula (I) in which G2 is a sulfur atom, G3 is an oxygen atom, R and R1 are hydrogen atoms and R2 and R3, which are the same or different, represent a CO—R5 or CO—(CH₂)₂ₙ₊₁—X—R6 group;
j) reacting a compound represented by general formula (I) in which G2 is a sulfur atom, G3 is an oxygen atom, R and R1 are hydrogen atoms and R2 and R3, which are the same or different, represent a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

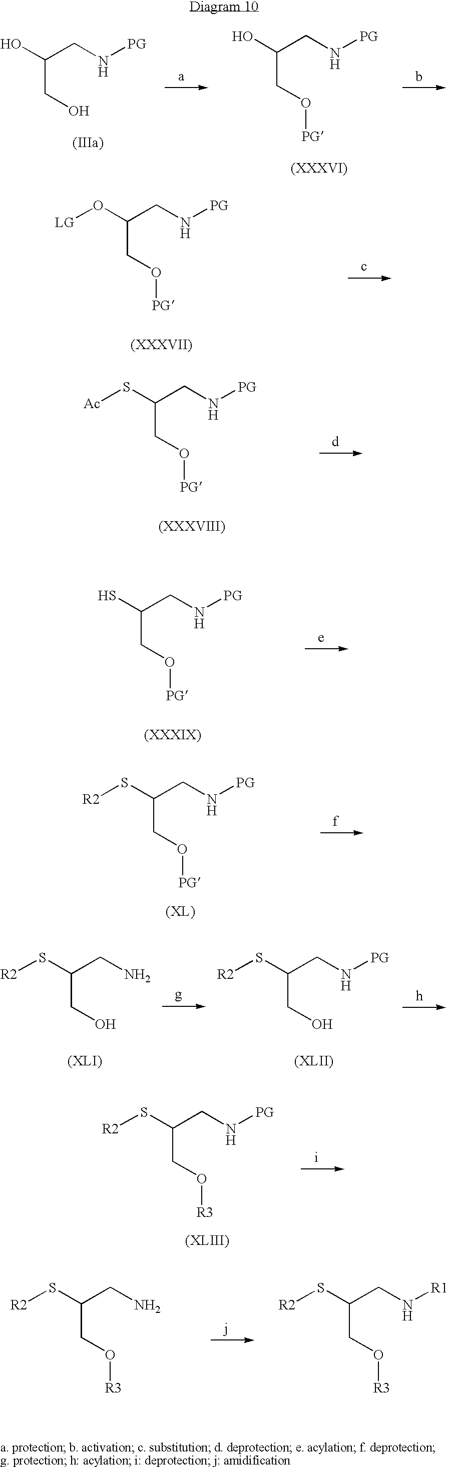

Compounds represented by formula (I) according to the invention in which (i) G2 is an oxygen atom, (ii) G3 is a sulfur atom, (iii) R is a hydrogen atom, (iv) R1 and R3 are hydrogen atoms or represent a CO—R5 or CO—(CH₂)₂ₙ₊₁—X—R6 group and (v) R2 represents a hydrogen atom or a CO—R5 or CO—(CH₂)₂ₙ₊₁—X—R6 group, can be prepared from compounds having formula (IIa) according to the following method (diagram 11):
a) reacting a compound represented by formula (IIa) such as defined hereinabove, with a compound corresponding to the formula LG-E (in stoichiometric amounts) in which E represents a halogen and LG is a reactive group selected for example in the group consisting of mesyl, tosyl, etc., to give a compound represented by general formula (XLIV);
b) reacting a compound represented by formula (XLIV) with a compound corresponding to the formula Ac—S^{—}B⁺ in which Ac represents a short acyl group, preferably the acetyl group, and B is a counter-ion selected for example in the group consisting of sodium and potassium, preferably potassium to give the compound represented by general formula (XLV). Advantageously, said reaction can be carried out by adapting the protocol described by (Gronowitz, Herslöf et al. 1978);
c) reacting a compound-represented by formula (XLV) with a compound PG-E in which PG is a protective group and E is a reactive group selected for example in the group consisting of OH and a halogen, to give a compound represented by general formula (XLVI). Advantageously, the reaction can be carried out by adapting the protocols described by (Marx, Piantadosi et al. 1988) and (Gaffney and Reese 1997), in which PG-E can represent triphenylmethyl chloride or 9-phenylxanthene-9-ol or else 9—Chloro-9-phenylxanthene;
d) deprotecting the sulfur atom of a compound (XLVI), in conditions known to those skilled in the art, to give a compound represented by general formula (XLVII);
e) reacting a compound represented by general formula (XLVII) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (XLVIII) in which R1 and R3, which are the same or different, represent a CO—R5 or CO—(CH₂)₂ₙ₊₁—X—R6 group;
f) deprotecting a compound represented by formula (XLVIII), in conventional conditions known to those skilled in the art, to give a compound represented by general formula (I) in which G2 is an oxygen atom, G3 is a sulfur atom, R and R2 are hydrogen atoms and R1 and R3, which are the same or different, represent a CO—R5 or CO—(CH₂)₂ₙ₊₁—X—R6 group;
g) reacting a compound represented by general formula (I) in which G2 is an oxygen atom, G3 is a sulfur atom, R and R2 are hydrogen atoms and R1 and R3, which are the same or different, represent a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

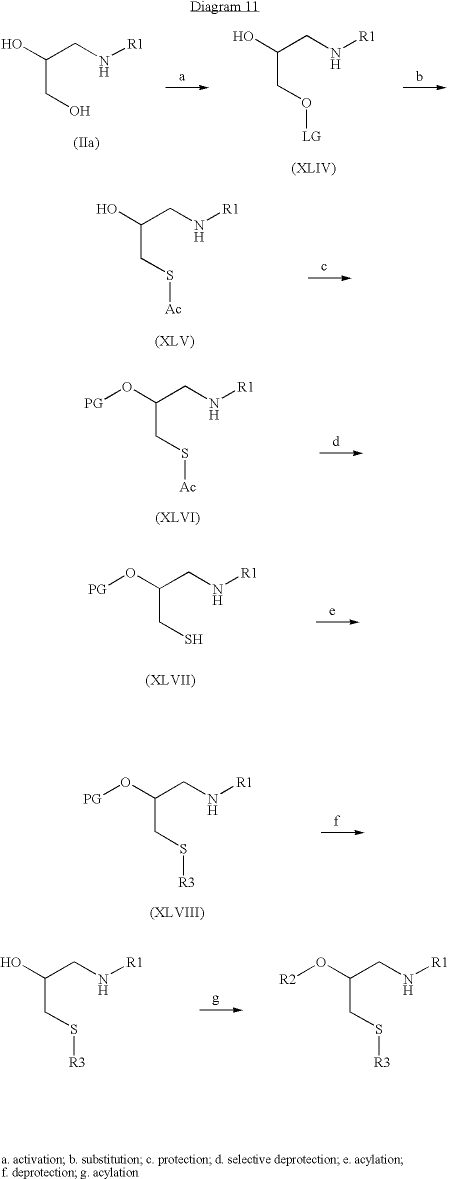

Compounds represented by formula (I) according to the invention in which (i) G2 is an oxygen atom, (ii) G3 is a sulfur atom, (iii) R is a hydrogen atom, (iv) R1 and R3 are hydrogen atoms or represent a CO—R5 or CO—(CH₂)₂ₙ₊₁—X—R6 group, which are the same or different, and (v) R3 represents a CO—R5 or CO—(CH₂)₂ₙ₊₁—X—R6 group, can be rpepared from compounds having formula (Ilia) according to the following method (diagram 12):
a) reacting a compound represented by formula (IIIa) such as defined hereinabove, with a compound corresponding to the formula LG-E (in stoichiometric amounts) in which E represents a halogen and LG is a reactive group selected for example in the group consisting of mesyl, tosyl, etc., to give a compound represented by general formula (XLIX);
b) reacting a compound represented by formula (XLIX) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (L). Advantageously, said reaction can be carried out by adapting the protocol described by (Gronowitz, Herslöf et al. 1978);
c) reacting a compound represented by formula (L) with a compound PG′-E in which PG′ is a protective group and E is a reactive group selected for example in the group consisting of OH and a halogen, to give a compound represented by general formula (LI). Advantageously, the reaction can be carried out by adapting the protocols described by (Marx, Piantadosi et al. 1988) and (Gaffney and Reese 1997) in which PG′-E can represent triphenylmethyl chloride or 9-phenylxanthene-9-ol or else 9—Chloro-9-phenylxanthene;
d) deprotecting the sulfur atom of a compound (LI), in conditions known to those skilled in the art, to give a compound represented by general formula (LII);
e) reacting a compound represented by general formula (LII) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LIII) in which R3 represents a CO—R5 or CO—(CH₂)₂ₙ₊₁—X—R6 group;
f) deprotecting a compound represented by formula (LIII), in conventional conditions known to those skilled in the art, to give a compound represented by general formula (I) in which G2 is an oxygen atom, G3 is a sulfur atom, R and R2 are hydrogen atoms and R3 represents a CO—R5 or CO—(CH₂)₂ₙ₊₁—X—R6 group (compound LIV);
g) reacting a compound represented by formula (LIV) with a compound (PG)₂O in which PG is a protective group to give a compound represented by general formula (LV). Advantageously, the reaction can be carried out by adapting the protocols described by (Nazih, Cordier et al. 2000) and (Kotsovolou, Chiou et al. 2001) in which (PG)₂O represents di-tert-butyl dicarbonate;
h) reacting a compound represented by general formula (LV) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by formula (LVI);
i) deprotecting a compound (LVI) in conventional conditions known to those skilled in the art to give a compound represented by general formula (I) in which G3 is a sulfur atom, G2 is an oxygen atom, R and R1 are hydrogen atoms and R2 and R3, which are the same or different, represent a CO—R5 or CO—(CH₂)₂ₙ₊₁—X—R6 group;
j) reacting a compound represented by general formula (I) in which G3 is a sulfur atom, G2 is an oxygen atom, R and R1 are hydrogen atoms and R2 and R3, which are the same or different, represent a CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

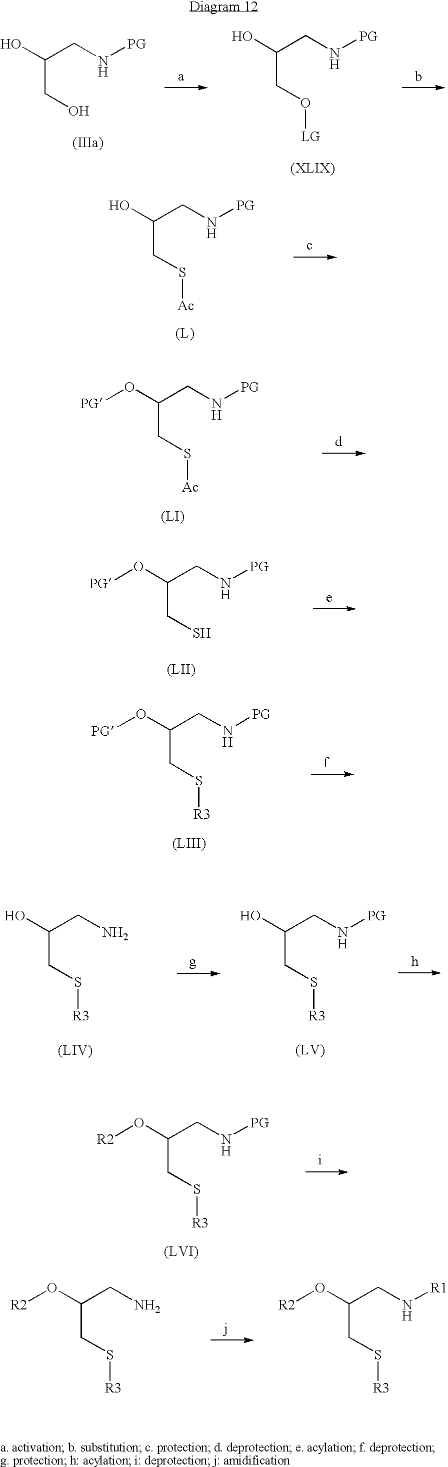

Compounds represented by formula (I) according to the invention in which (i) G2 is an oxygen atom, (ii) G3 is a sulfur atom, (iii) R is a hydrogen atom, (iv) R2 and R3, which are the same, are hydrogen atoms or represent a CO—R5 or CO—(CH₂)₂ₙ₊₁—X—R6 group and (v) R1 represents a CO—R5 or CO—(CH₂)₂ₙ₊₁—X—R6 group, can be prepared from compounds having formula (IIIa) according to the following method (diagram 13):
a) reacting a compound represented by formula (IIIa) such as defined hereinabove, with a compound corresponding to the formula (LG)2 (in stoichiometric amounts) in which LG is a reactive group selected for example in the group consisting of iodine, bromine, etc., to give a compound represented by general formula (XLIXa);
b) reacting a compound represented by formula (XLIXa) with a compound corresponding to the formula Ac—S⁻B⁺ in which Ac represents a short acyl group, preferably the acetyl group, and B is a counter-ion selected for example in the group consisting of sodium and potassium, preferably potassium to give the compound represented by general formula (L);
c) deprotecting the sulfur atom of a compound (L), in conditions known to those skilled in the art, to give a compound represented by general formula (LVII);
d) reacting a compound represented by general formula (LVII) with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art to give a compound represented by general formula (LVI) in which R2 and R3 represent a same CO—R5 or CO—(CH₂)₂ₙ₊₁—X—R6 group;
e) deprotecting a compound represented by formula (LVI), in conventional conditions known to those skilled in the art, to give a compound represented by general formula (I) in which G2 is an oxygen atom, G3 is a sulfur atom, R and R2 are hydrogen atoms and R2 and R3 represent a same CO—R5 or CO—(CH₂)₂ₙ₊₁—X—R6 group;
f) reacting a compound represented by general formula (I) in which G2 is an oxygen atom, G3 is a sulfur atom, R and R2 are hydrogen atoms and R2 and R3 represent a same CO—R5 or CO—(CH₂)₂ₙ₊₁—X—R6 group with a compound corresponding to the formula A°—CO-A2 in which A2 is a reactive group selected for example in the group consisting of OH and Cl, and A° is the R5 group or the (CH₂)₂ₙ₊₁—X—R6 group, possibly in the presence of coupling agents or activators known to those skilled in the art.

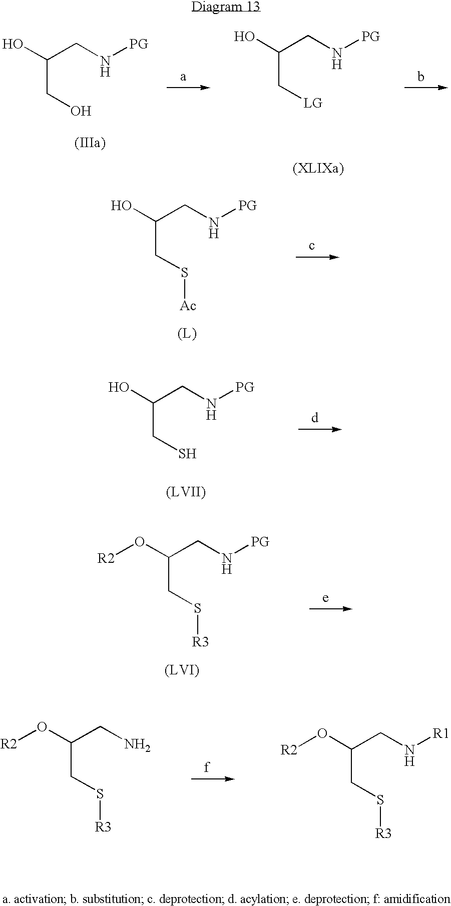

The feasibility, realization and other advantages of the invention are further detailed in the following examples, which are given for purposes of illustration and not by way of limitation.

### LEGENDS OF FIGURES

FIG. 1: Structure of particular inventive compounds whose preparation is described in examples 2, 4, 5, 6, 8, 10 to 14, 16, 18, 19, 21 and 23, and respectively noted on the figure as 1A.2, 1A.4, 1A.5, 1A.6, 1A.8, 1A.10, 1A.11, 1A.12, 1A.13, 1A.14, 1A.16, 1A.18, 1A.19, 1A.21 and 1A.23.

FIG. 2: Evaluation of the PPARα agonist properties of the inventive compounds with the Gal4/PPARα transactivation system

FIG. 3: Evaluation of the effects of an inventive compound (example 11) on plasma cholesterol and triglyceride metabolism in Zucker rats.

FIG. 3A: assay of total plasma cholesterol at D0, D7 and D14 in control animals and in animals treated with compound Ex 11.

FIG. 3B: assay of plasma triglycerides at D0, D7 and D14 in control animals and in animals treated with compound Ex 11.

FIG. 4: Evaluation of the antioxidant properties of the inventive compounds on LDL oxidation by copper (Cu).

FIG. 4a: conjugated diene formation over time or lag phase.

FIG. 4b: LDL oxidation rate.

FIG. 4c: maximum amount of conjugated dienes formed.

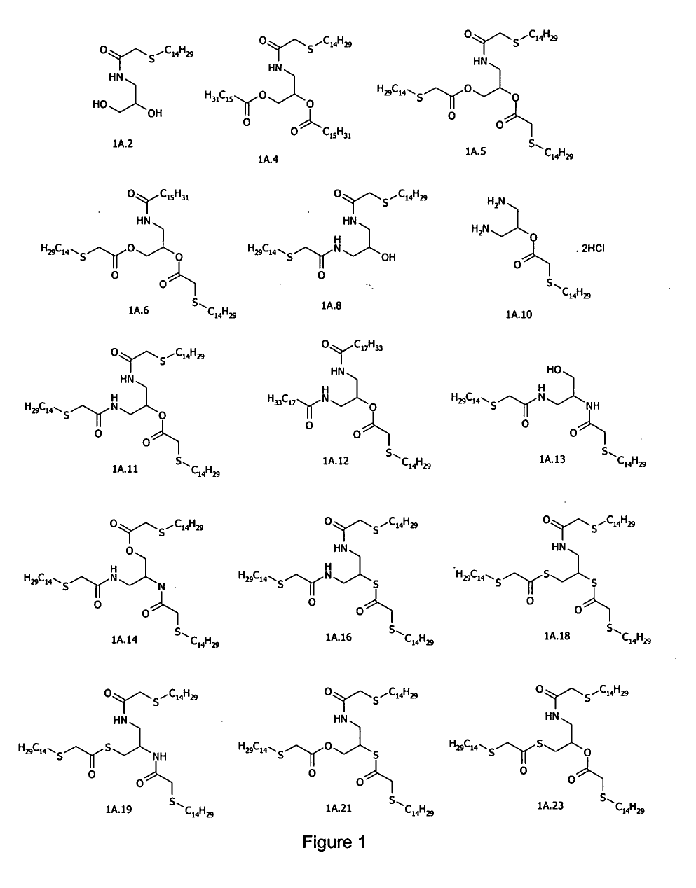

### EXAMPLES

For easier comprehension of the text, the inventive compounds used in the examples concerning the measurement and evaluation of activity are abbreviated as follows: “Ex 2”, for instance, indicates the inventive compound whose preparation is described in example 2.

Thin-layer chromatography (TLC) was carried out on plates coated with MERCK silica gel 60F₂₅₄ 0.2 mm thick. Retention factor is abbreviated Rf.

Column chromatography was carried out on silica gel 60 with a particle size of 40-63 μm (Merck reference 9385-5000).

Melting points (MP) were determined on a Buchi B 540 apparatus by the capillary method.

Infrared (IR) spectra were recorded on a Bruker Fourier transformation spectrometer (Vector 22).

Nuclear magnetic resonance (NMR) spectra were recorded on a Bruker AC 300 spectrometer (300 MHz). Each signal was identified by its chemical shift, intensity, multiplicity (noted s for singlet, sl for broad singlet, d for doublet, dd for split doublet, t for triplet, td for split triplet, quint for quintuplet and m for multiplet) and its coupling constant (J).

Mass spectra (MS) were determined on a Perkin Elmer Sciex API 1 (ESI-MS for ElectroSpray Ionization Mass Spectrometry) or on an Applied Biosystems Voyager DE-STR of the MALDI-TOF type (Matrix-Assisted Laser Desorption/Ionization—Time Of Flight).

### Example 1

### Preparation of tetradecylthioacetic acid

Potassium hydroxide (34.30 g, 0.611 mol), mercaptoacetic acid (20.9 ml, 0.294 mol) and 1-bromotetradecane (50 ml, 0.184 mol) were added in that order to methanol (400 ml). The mixture was stirred overnight at room temperature. A concentrated hydrochloric acid solution (60 ml) dissolved in water (800 ml) was then added. The tetradecylthioacetic acid precipitated. The mixture was stirred overnight at room temperature. The precipitate was then filtered, washed five times with water and dried in a dessicator. The product was recrystallized in methanol.

Yield: 94%

Rf (dichloromethane/methanol 9:1): 0.60

MP: 67-68° C.

IR: νCO acid 1726 and 1684 cm⁻¹

NMR (¹H, CDCl₃): 0.84-0.95 (t, 3H, —CH₃, J=6.5 Hz); 1.20-1.45 (multiplet, 22H, —CH₂—); 1.55-1.69 (quint, 2H, —CH₂—CH₂—S—, J=6.5 Hz); 2.63-2.72 (t, 2H, CH₂—CH₂—S—, J=7.3 Hz); 3.27 (s, 2H, S—CH₂—COOH)

MS (ESI-MS): M−1=287

### Example 2

### Preparation of 3-(tetradecylthioacetylamino)propane-1,2-diol

Tetradecylthioacetic acid (example 1) (14.393 g, 50 mmol) and 3-amino-propane-1,2-diol (5 g, 55 mmol) were placed in a flask and heated at 190° C. for 1 hour. The reaction mixture was cooled to room temperature, taken up in chloroform and washed once with water. The organic phase was dried on magnesium sulfuate, filtered and dried. The residue was stirred in ether and the product was isolated by filtration.

Yield: 22%

Rf (dichloromethane/methanol 9:1): 0.60

MP: 89-92° C.

IR: νNH and OH 3282 cm⁻¹; νCO amide 1640 cm⁻¹

NMR (¹H, CDCl₃): 0.89 (t, 3H, —CH₃, J=6.5 Hz); 1.26 (multiplet, 22H, —CH₂—); 1.57 (m, 2H, —CH₂—CH₂—S—); 2.54 (t, 2H, —CH₂—CH₂—S, J=7.6 Hz); 3.27 (s, 2H, S—CH₂—CONH—); 3.47 (m, 2H, —CONH—CH₂—CHOH—CH₂OH); 3.58 (m, 1H, —CONH—CH₂—CHOH—CH₂OH);3.81 (m, 2H, —CONH—CH₂—CHOH—CH₂OH); 7.33 (sl, 1H, —CONH).

MS (MALDI-TOF): M+1=362 (M+H); M+23=385 (M+Na⁺); M+39=400 (M+K⁺)

### Example 3

### 3-(palmitoylamino)propane-1,2-diol

This compound was synthesized according to the method described hereinabove (example 2) from 3-amino-propane-1,2-diol and palmitic acid.

Yield: 86%

Rf (dichloromethane/methanol 9:1): 0.50

IR: νNH and OH 3312 cm⁻¹; νCO amide 1633 cm⁻¹

MP: 104-108° C.

NMR (¹H, CDCl₃): 0.89 (t, 3H, —CH₃, J=6.5 Hz); 1.28 (multiplet, 24H, —CH₂—); 1.64 (m, 2H, —CH₂—CH₂—CO—); 2.24 (m, 2H, —CH₂—CH₂—CO—); 3.43 (m, 2H, —CONH—CH₂—CHOH—CH₂OH); 3.55 (m, 2H, —CONH—CH₂—CHOH—CH₂OH); 3.78 (m, 1H, —CONH—CH₂—CHOH—CH₂OH); 5.82 (sl, 1H, —CONH—).

MS (MALDI-TOF): M+1=330 (M+H)

### Example 4

### Preparation of 1,2-(dipalmitoyloxy)-3-tetradecylthioacetylaminopropane

3-(tetradecylthioacetylamino)propane-1,2-diol (example 2) (1 g, 2.77 mmol) was dissolved in dichloromethane (200 ml). Dicyclohexylcarbodiimide (1.426 g, 6.91 mmol), dimethylaminopyridine (0.845 g, 6.91 mmol) and palmitic acid (1.773 g, 6.91 mmol) were then added and the mixture was stirred at room temperature for 48 hours. The dicyclohexylurea which precipitated was filtered and washed with dichloromethane. The filtrate was vacuum evaporated. The residue was purified by chromatography on silica gel (eluent:dichloromethane/cyclohexane 6:4).

Yield: 28%

Rf (dichloromethane/cyclohexane 7:3): 0.28

MP: 73-75° C.

IR: νNH 3295 cm⁻¹; νCO ester 1730 cm⁻¹; νCO amide 1663 cm⁻¹

NMR (¹H, CDCl₃): 0.89 (t, 9H, —CH₃, J=6.5 Hz); 1.26 (multiplet, 70H, —CH₂—); 1.57 (multiplet, 6H, —CH₂—CH₂—S— and OCOCH₂—CH₂); 2.33 (t, 4H, OCOCH₂—CH₂—, J=7.3 Hz); 2.51 (t, 2H, CH₂—CH₂—S—, J=7.3 Hz); 3.22 (s, 2H, S—CH₂—CONH—); 3.47 (m, 1H, —CONH—CHaHb-CH—CHcHd-); 3.62 (m, 1H, —CONH—CHaHb-CH—CHcHd); 4.12 (dd, 1H, —CHaHb-CH—CHcHd-, J=12.1 Hz and J=5.7 Hz); 4.36 (dd, 1H, —CHaHb-CH—CHcHd-, J=12.1 Hz and J=4.4 Hz); 5.15 (m, 1H, —CHaHb-CH—CHaHb); 7.20 (m, 1H, —NHCO—).

MS (MALDI-TOF): M+1=838 (M+H); M+23=860 (M+Na⁺); M+39=876 (M+K⁺⁾

### Example 5

### Preparation of 1,2-(ditetradecylthioacetyloxy)-3-tetradecylthioacetylaminopropane

This compound was synthesized according to the method described hereinabove (example 4) from 3-(tetradecylthioacetylamino)propane-1,2-diol (example 2) and tetradecylthioacetic acid (example 1).

Yield: 41%

Rf (dichloromethane): 0.23

IR: νNH 3308 cm⁻¹; νCO ester 1722 and 1730 cm⁻¹; νCO amide 1672 cm⁻¹

MP: 65-67° C.

NMR (¹H, CDCl₃): 0.89 (t, 9H, —CH₃, J=6.4 Hz); 1.26 (multiplet, 66H, —CH₂—); 1.59 (multiplet, 6H, —CH₂—CH₂—S—); 2.53 (t, 2H, —CH₂—CH₂—S—CH₂—CONH—, J=7.3 Hz); 2.64 (t, 4H, CH₂—CH₂—S—CH₂—COO—, J=7.3 Hz); 3.23 (s, 4H, S—CH₂—COO—); 3.24 (s, 2H, S—CH₂—CONH—); 3.52 (m, 1H, —CONH—CHaHb-CH—CHcHd-); 3.67 (m, 1H, —CONH—CHaHb-CH—CHcHd-); 4.22 (dd, 1H, —CHaHb-CH—CHcHd-, J=12.2 Hz and J=5.4 Hz); 4.36 (dd, 1H, —CHaHb-CH—CHcHd-, J=12.2 Hz and J=3.9 Hz); 5.19 (m, 1H, —CHaHb-CH—CHaHb-); 7.18 (m, 1H, —NHCO—).

MS (MALDI-TOF): M+1=902 (M+H); M+23=924 (M+Na⁺); M+39=940 (M+K⁺)

### Example 6

### Preparation of 1,2-(ditetradecylthioacetyloxy)-3-palmitoylaminopropane

This compound was synthesized according to the method described hereinabove (example 4) from 3-(palmitoylamino)propane-1,2-diol (example 3) and tetradecylthioacetic acid (example 1).

Yield: 8%

Rf (ethyl acetate/cyclohexane 2:8): 0.33

IR: νNH 3319 cm⁻¹; νCO ester 1735 cm⁻¹; νCO amide 1649 cm⁻¹

MP: 82-83° C.

NMR (¹H, CDCl₃): 0.89 (t, 9H, —CH₃, J=6.4 Hz); 1.26 (multiplet, 68H, —CH₂—); 1.60 (multiplet, 6H, —CH₂—CH₂—S— and —CH₂—CH₂—CONH—); 2.18 (t, 2H, —CH₂—CH₂—CONH—, J=6.8 Hz); 2.64 (multiplet, 4H, CH₂—CH₂—S—CH₂—COO—); 3.22 (s, 2H, —S—CH₂—COO—); 3.24 (s, 2H, —S—CH₂—COO—); 3.47 (m, 1H, —CONH—CHaHb-CH—CHcHd-); 3.62 (m, 1H, —CONH—CHaHb-CH—CHcHd-); 4.23 (dd, 1H, —CHaHb-CH—CHcHd-, J=11.9 Hz and J=5.6 Hz); 4.36 (dd, 1H, —CHaHb-CH—CHcHd-, J=12.2 Hz and J=4 Hz); 5.15 (m, 1H, —CHaHb-CH—CHaHb-); 5.85 (m, 1H, —NHCO—).

MS (MALDI-TOF): M+1=870 (M+H)

### Example 7

### Preparation of 1,3-di(oleylamino)propan-2-ol

Oleic acid (5.698 g, 0.020 mol) and 1,3-diaminopropan-2-ol (1 g, 0.011 mol) were placed in a flask and heated at 190° C. for 2 hours. The reaction mixture was cooled to room temperature, then taken up in chloroform and washed with water. The aqueous phase was extracted with chloroform and the organic phases were combined, dried on magnesium sulfate, filtered and evaporated to dryness to yield an oily black residue (6.64 g) which was purified by chromatography on silica gel (eluent:dichloromethane/methanol 99:1). The resulting product was then washed with ether and filtered.

Yield: 23%

Rf (dichloromethane/methanol 95:5): 0.43

IR: νNH 3306 cm⁻¹; νCO amide 1646 and 1630 cm⁻¹

MP: 88-92° C.

NMR (¹H, CDCl₃): 0.89 (t, 6H, —CH₃, J=6.2 Hz); 1.28 (multiplet, 68H, —CH₂—); 1.61-1.66 (multiplet, 4H, —CH₂—CH₂—CONH—); 1.98-2.02 (multiplet, 8H, —CH₂—CH═CH—CH₂—); 2.23 (t, 4H, —CH₂—CH₂—CONH—, J=7.0 Hz); 3.25-3.42 (multiplet, 4H, —CONH—CH₂—CH—CH₂—); 3.73-3.80 (m, 1H, —CONH—CH₂—CH—CH₂—); 5.30-5.41 (multiplet, 4H, —CH₂—CH═CH—CH₂—); 6.36 (multiplet, 2H, —NHCO—).

MS (MALDI-TOF): M+1=619 (M+H⁺); M+23=641 (M+Na⁺); M+39=657 (M+K⁺)

### Example 8

### Preparation of 1,3-di(tetradecylthioacetylamino)propan-2-ol

This compound was synthesized according to the method described hereinabove (example 7) from 1,3-diaminopropan-2-ol and tetradecylthioacetic acid (example 1).

Yield: 94%

Rf (dichloromethane/methanol 95:5): 0.44

IR: νNH 3275 cm⁻¹; νCO amide 1660 and 1633 cm⁻¹

MP: 101-104° C.

NMR (¹H, CDCl₃): 0.89 (t, 6H, —CH₃, J=6.3 Hz); 1.28 (multiplet, 44H, —CH₂—); 1.57-1.62 (multiplet, 4H, —CH₂—CH₂—S—CH₂—CONH—); 2.55 (t, 4H, —CH₂—CH₂—S—CH₂—CONH—, J=7.2 Hz); 3.26 (s, 4H,—S—CH₂—CONH—); 3.32-3.36 (multiplet, 2H, —CONH—CHₐH_{b}—CH—CHₐH_{b}—NHCO—); 3.43-3.49 (multiplet, 2H, —CONH—CHₐH_{b}—CH—CHₐH_{b}—NHCO—); 3.82-3.84 (m, 1H, —CONH—CH₂—CH—CH₂—NHCO—); 7.44 (sl, 2H, —NHCO).

MS (MALDI-TOF): M+23=653 (M+Na⁺); M+39=669 (M+K⁺)

### Example 9

### Preparation of 1,3-di(stearoylamino)propan-2-ol

This compound was synthesized according to the method described hereinabove (example 7) from 1,3-diaminopropan-2-ol and stearic acid.

Yield: 73%

Rf (dichloromethane/methanol 95:5): 0.28

IR: νNH 3306 cm⁻¹; νCO amide 1647 and 1630 cm⁻¹

MP: 123-130° C.

MS (MALDI-TOF): M+23=645 (M+Na⁺)

### Example 10

### Preparation of 1,3-diamino-2-(tetradecylthioacetyloxy)propane dihydrochloride

### Preparation of 1,3-di(tert-butyloxycarbonylamino)propan-2-ol (example 10a)

1,3-diaminopropan-2-ol (3 g, 0.033 mol) was dissolved in methanol (300 ml) followed by the addition of triethylamine (33 ml dropwise) and di-tert-butyl dicarbonate [(BOC)₂O] (21.793 g, 0.100 mol) wherein BOC corresponds to tertbutyloxycarbonyl. The reaction medium was heated at 40-50° C. for 20 min then stirred at room temperature for 1 hour. After evaporation of the solvent, the colorless oily residue was purified by chromatography on silica gel (eluent:dichloromethane/methanol 95:5). The reaction yielded a colorless oil which crystallized slowly.

Yield: quantitative

Rf (dichloromethane/methanol 95:5): 0.70

IR: νNH 3368 cm⁻¹; νCO carbamate 1690 cm⁻¹

MP: 98-100° C.

NMR (¹H, CDCl₃): 1.45 (multiplet, 18H, —CH₃— (BOC)); 3.02 (sl, 1H, OH); 3.15-3.29 (multiplet, 4H, BOCNH—CH₂—CH—CH₂—NHBOC); 3.75 (m, 1H, BOCNH—CH₂—CH—CH₂—NHBOC); 5.16 (multiplet, 2H, —NHBOC).

MS (MALDI-TOF): M+1=291 (M+H⁺); M+23=313 (M+Na⁺); M+39=329 (M+K⁺)

### Preparation of 1,3-di(tert-butyloxycarbonylamino)-2-(tetradecylthioacetyloxy)-propane (example 10b)

1,3-(di-tert-butoxycarbonylamino)-propan-2-ol (example 10a) (1 g, 3.45 mmol), tetradecylthioacetic acid (example 1) (0.991 g, 3.45 mmol) and dimethylaminopyridine (0.042 g, 0.34 mmol) were dissolved in dichloromethane (40 ml) at 0° C. Dicyclohexylcarbodiimide (0.709 g, 3.45 mmol) diluted in dichloromethane was then added dropwise and the mixture was stirred at 0° C. for 30 min, then brought to room temperature. After 20 hours of reaction, the dicyclohexylurea precipitate was filtered and the filtrate was dried. The oily residue was purified by chromatography on silica gel (eluent:dichloromethane/cyclohexane 5:5 followed by dichloromethane/ethyl acetate 98:2).

Yield: 52%

Rf (dichloromethane/ethyl acetate 95:5): 0.43

IR: νNH 3369 cm⁻¹; νCO carbamate 1690 cm⁻¹; νCO ester 1719 cm⁻¹

NMR (¹H, CDCl₃): 0.89 (t, 3H, CH₃, J=6.3 Hz); 1.26 (multiplet, 22 H, —CH₂—); 1.45 (multiplet, 18H, —CH₃— (BOC)); 1.56-1.66 (m, 2H, —CH₂—CH₂—S—CH₂—CO); 2.64 (t, 2H, —CH₂—CH₂—S—CH₂—CO, J=7.5 Hz); 3.20 (s, 2H, CH₂—S—CH₂—CO); 3.35 (multiplet, 4H, BOCNH—CH₂—CH—CH₂—NHBOC); 4.89 (m, 1H, BOCNH—CH₂—CH—CH₂—NHBOC); 5.04 (multiplet, 2H, —NHBOC).

MS (MALDI-TOF): M+23=583 (M+Na⁺); M+39=599 (M+K⁺)

### Preparation of 1,3-diamino-2-(tetradecylthioacetyloxy)propane dihydrochloride (example 10)

1,3-(ditert-butoxycarbonylamino)-2-tetradecylthioacetyloxypropane (example 10b) (0.800 g, 1.43 mmol) was dissolved in diethyl ether (50 ml) saturated with gaseous hydrochloric acid. The reaction medium was stirred at room temperature for 20 hours. The precipitate which formed was then filtered and washed with ether. The product was obtained as the dihydrochloride.

Yield: 88%

Rf (dichloromethane/methanol 7:3): 0.37

IR: νNH₂ 3049 and 3099 cm⁻¹; νCO ester 1724 cm⁻¹

MP: 224° C. (decomposition)

NMR (¹H, CDCl₃): 0.86 (t, 3H, CH₃, J=6.3 Hz); 1.24 (multiplet, 22 H, —CH₂—); 1.48-1.55 (m, 2H, —CH₂—CH₂—S—CH₂—CO); 2.57 (t, 2H, —CH₂—CH₂—S—CH₂—CO, J=7.2 Hz); 3.16 (multiplet, 4H, BOCNH—CH₂—CH—CH₂—NH); 3.56 (s, 2H, CH₂—S—CH₂—CO); 5.16 (m, 1H, BOCNH—CH₂—CH—CH₂—NH); 8.43 (multiplet, 6H, —NH₂.HCl).

MS (MALDI-TOF): M+1=361 (M+H⁺); M+23=383 (M+Na⁺); M+39=399 (M+K⁺)

### Example 11

### Preparation of 1,3-ditetradecylthioacetylamino-2-(tetradecylthioacetyloxy)propane

1,3-diamino-2-tetradecylthioacetyloxypropane dihydrochloride (example 10) (0.400 g, 0.92 mmol) and tetradecylthioacetic acid (example 1) (0.532 g, 1.84 mmol) were dissolved in dichloromethane (50 ml) at 0° C. followed by the addition of triethylamine (0.3 ml, 2.1 mmol), dicyclohexylcarbodiimide (0.571 g, 2.77 mmol) and hydroxybenzotriazole (HOBT) (0.249 g, 1.84 mmol). The reaction medium was stirred at 0° C. for 1 hour then brought to room temperature for 48 hours. The dicyclohexylurea precipitate was filtered and washed with dichloromethane. The filtrate was vacuum evaporated. The residue obtained (1.40 g) was purified by chromatography on silica gel (eluent:dichloromethane followed by dichloromethane/ethyl acetate 9:1).

Yield: 74%

Rf (dichloromrthane/ethyl acetate 8:2): 0.25

IR: νNH 3279, 3325 cm⁻¹; νCO ester 1731 cm⁻¹; νCO amide 1647, 1624 cm⁻¹

MP: 87-89° C.

NMR (¹H, CDCl₃): 089 (t, 9H, CH₃, J=6.6 Hz); 1.26 (multiplet, 66H, —CH₂—); 1.55-1.60 (multiplet, 6H, —CH₂—CH₂—S—CH₂—CO); 2.55 (t, 4H, —CH₂—CH₂—S—CH₂—CONH—, J=7.2 Hz); 2.65 (t, 2H, —CH₂—CH₂—S—CH₂—COO—, J=7.2 Hz); 3.21 (s, 2H, —CH₂—S—CH₂—COO—); 3.25 (s, 4H, —CH₂—S—CH₂—CONH—); 3.40-3.49 (m, 2H, —CONH—CHₐH_{b}—CH—CHₐH_{b}—NHCO—); 3.52-3.61 (m, 2H, —CONH—CHₐH_{b}—CH—CHₐH_{b}—NHCO—); 4.96 (m, 1H, —CONH—CH₂—CH—CH₂—NHCO—); 7.42 (multiplet, 2H, —NHCO—).

MS (MALDI-TOF): M+1=901 (M+H⁺); M+23=923 (M+Na⁺); M+39=939 (M+K⁺)

### Example 12

### Preparation of 1,3-dioleylamino-2-(tetradecylthioacetyloxy)propane

This compound was synthesized according to the method described in example 11 from 1,3-diamino-2-tetradecylthioacetyloxypropane dihydrochloride (example 10) and oleic acid.

Yield: 15%

Rf (dichloromethane/ethyl acetate 8:2): 0.38

IR: νNH 3325 cm⁻¹; νCO ester 1729 cm⁻¹; νCO amide 1640 and 1624 cm⁻¹

MP: 57-59° C.

NMR (¹H, CDCl₃): 0.89 (t, 9H, CH₃, J=6.6 Hz); 1.26 (multiplet, 62H, —CH₂—); 1.59-1.74 (multiplet, 6H, —CH₂—CH₂—S—CH₂—CO); 1.92-2.03 (multiplet, 8H, —CH₂—CH═CH—CH₂—); 2.22 (t, 4H, —CH₂—CH₂—S—CH₂—CONH, J=7.2 Hz); 2.65 (t, 2H, —CH₂—CH₂—S—CH₂—COO—, J=7.4 Hz); 3.19 (s, 2H, —CH₂—S—CH₂—COO—); 3.25-3.34 (m, 2H, —CONH—CHₐH_{b}—CH—CHₐH_{b}—NHCO—); 3.56-3.65 (m, 2H, —CONH—CHₐH_{b}—CH—CHₐH_{b}—NHCO—); 4.87 (m, 1H, —CONH—CH₂—CH—CH₂—NHCO—); 5.34 (multiplet, 4H, —CH₂—CH═CH—CH₂—); 6.27 (multiplet, 2H, —NHCO—).

MS (MALDI-TOF): M+1=889 (M+H⁺); M+23=912 (M+Na⁺)

### Example 13

### Preparation of 2,3-ditetradecylthioacetylaminopropan-1-ol

### Preparation of methyl 2.3-diaminopropanoate dihydrochloride (example 13a)

2,3-diaminopropionic acid hydrochloride (1 g, 7 mmol) was dissolved in methanol (40 ml). The medium was cooled in an ice bath, followed by dropwise addition of thionyl chloride (2.08 ml, 28 mmol). The medium was brought to room temperature then refluxed for 20 hours. The solvent was evaporated and the residue was triturated in heptane. The resulting precipitate was filtered, washed and dried to give a yellowish-white solid.

Yield: 94%

Rf: (dichloromethane/methanol 9:1): 0.03

IR: νNH₂ 2811 cm⁻¹; νCO ester 1756 cm⁻¹

MP: 170-180° C. (decomposition)

NMR (¹H, CDCl₃): 3.78 (s, 3H, —CH₃); 4.33 (m, 3H, —CH₂— et —CH—); 8.77 (m, 3H, —NH₂.HCl); 9.12 (m, 3H, —NH₂.HCl)

### Preparation of methyl 2,3-ditetradecylthioacetylaminopropanoate (example 13b)

Methyl 2,3-diaminopropanoate dihydrochloride (example 13a) (0.500 g, 2.62 mmol) and tetradecylthioacetic acid (example 1) (1.51 g, 5.23 mmol) were dissolved in dichloromethane (80 ml) at 0° C. followed by the addition of triethylamine (0.79 ml), dicyclohexylcarbodiimide (1.62 g, 7.85 mmol) and hydroxybenzotriazole (0.707 g, 5.23 mmol). The reaction medium was stirred at 0° C. for 1 hour then brought to room temperature for 48 hours. The dicyclohexylurea precipitate was filtered and washed with dichloromethane and the filtrate was evaporated. The residue obtained (3.68 g) was purified by chromatography on silica gel (eluent:dichloromethane/ethyl acetate 95:5) to give the desired compound in the form of a white powder.

Yield: 96%

Rf: (dichloromethane/methanol 98:2): 0.63

IR: νNH amide 3276 cm⁻¹; νCO ester 1745 cm⁻¹; νCO amide 1649 cm⁻¹

MP: 81.5-82.5° C.

NMR (¹H, CDCl₃): 0.89 (t, 6H, CH₃, J=6.6 Hz); 1.26-1.37 (multiplet, 44H, —CH₂—); 1.56-1.61 (m, 4H, —CH₂—CH₂—S—CH₂—CONH); 2.50-2.60 (m, 4H, —CH₂—CH₂—S—CH₂—CONH—); 3.22 (s, 2H, —CH₂—S—CH₂—CONH—); 3.25 (s, 2H, —CH₂—S—CH₂—CONH—); 3.74 (m, 2H, H₃CO(CO)—CH—CH₂—NHCO—); 3.79 (s, 3H, —COOCH₃); 4.64-4.70 (m, 1H, H₃CO(CO)—CH—CH₂—NHCO—); 7.79 (d, 2H, —NHCO—, J=7.3 Hz).

MS (MALDI-TOF): M+1=659 (M+H⁺); M+23=681 (M+Na⁺); M+39=697 (M+K⁺)

### Preparation of 2,3-ditetradecylthioacetylaminopropan-1-ol (example 13)

Sodium borohydride (316 mg, 8.4 mmol) was dissolved in tetrahydrofuran (40 ml). The reaction mixture was cooled in an ice bath and methyl 2,3-ditetradecylthioacetylaminopropanoate (example 13b) (500 mg, 0.76 mmol) was then added in small portions. The mixture was brought to room temperature and stirred. After 4 days of reaction, 20 ml of water were added. The product, which precipitated, was filtered, washed with water then dried in a dessicator to give a white powder.

Yield: 76%

Rf: (dichloromethane/methanol 95:5): 0.53

IR: νOH alcohol 3436 cm⁻¹; νNH amide 3313 and 3273 cm⁻¹; νCO amide 1648 and 1622 cm⁻¹

MP: 100.2-102.2° C.

NMR (¹H, CDCl₃): 0.89 (t, 6H, CH₃, J=6.2 Hz); 1.26 (multiplet, 44H, —CH₂—); 1.59 (m, 4H, —CH₂—CH₂—S—CH₂—CONH); 2.50-2.56 (m, 4H, —CH₂—CH₂—S—CH₂—CONH—); 3.23 (s, 2H, —CH₂—S—CH₂—CONH—); 3.27 (s, 2H, —CH₂—S—CH₂—CONH—); 3.50-3.91 (multiplet, 5H, —OCO—CH₂—CH—CH₂—NHCO—); 7.38 (d, 2H, —NHCO—, J=7.1 Hz).

MS (MALDI-TOF): M+1=631 (M+H⁺); M+23=653 (M+Na⁺); M+39=669 (M+K⁺)

### Example 14

### Preparation of 2,3-ditetradecylthioacetylamino-1-tetradecylthioacetyloxypropane

2,3-ditetradecylthioacetylaminopropan-1-ol (example 13) (0.200 g, 0.32 mmol) was dissolved in tetrahydrofuran (40 ml) followed by the addition of dicyclohexylcarbodiimide (65 mg, 0.32 mmol), dimethylaminopyridine (39 mg, 0.32 mmol) and tetradecylthioacetic acid (example 1) (91 mg, 0.32 mmol). The mixture was stirred at room temperature for 20 hours. The dicyclohexylurea precipitate was filtered, washed with tetrahydrofuran and the filtrate was evaporated. The residue obtained (1 g) was purified by flash chromatography (eluent:dichloromethane) to produce the desired compound in the form of a white powder.

Yield: 59%

Rf: (dichloromethane/ethyl acetate 8:2): 0.49

IR: νNH amide 3281 cm⁻¹; νCO ester 1736 cm⁻¹; νCO amide 1641 cm⁻¹

MP: 95.4-97.3° C.

NMR (¹H, CDCl₃): 0.89 (t, 9H, CH₃, J=6.4 Hz); 1.27-1.34 (multiplet, 66H, —CH₂—); 1.54-163 (m, 6H, —CH₂—CH₂—S—CH₂—CO—); 2.53 (t, 4H, —CH₂—CH₂—S—CH₂—CONH—, J=7.2 Hz); 2.65 (t, 2H, —CH₂—CH₂—S—CH₂—COO—, J=7.2 Hz); 3.21 (s, 2H, —CH₂—S—CH₂—CONH—); 3.23 (s, 2H, —CH₂—S—CH₂—CONH—); 3.25 (s, 2H, —CH₂—S—CH₂—COO—); 3.46-3.56 (m, 2H, —OCO—CH₂—CH—CH₂—NHCO—); 4.22-4.25 (m, 2H, —OCO—CH₂—CH—CH₂—NHCO—); 4.29-4.39 (m, 1H, —OCO—CH₂—CH—CH₂—NHCO—); 7.29 (t, 1H, —NHCO—); 7.38 (d, 1H, —NHCO—, J=7.6 Hz).

MS (MALDI-TOF): M+1=901 (M+H⁺)

### Example 15

### Preparation of 1,3-diamino-2-(tetradecylthioacetylthio)propane dihydrochloride

### Preparation of 1,3-di(tert-butyloxycarbonylamino)-2-(p-toluenesulfonyloxy) propane (example 15a)

1,3-di(tert-butyloxycarbonylamino)propan-2-ol (example 10a) (2.89 g, 10 mmol) and triethylamine (2.22 ml, 16 mmol) were dissolved in anhydrous dichloromethane (100 ml). The reaction mixture was cooled in an ice bath followed by dropwise addition of tosyl chloride (2.272 g, 12 mmol) dissolved in dichloromethane (30 ml). The reaction mixture was then, stirred at room temperature for 72 hours. One equivalent of chloride and 1.6 of triethylamine were added after 48 hours. Water was added to stop the reaction and the medium was allowed to settle. The organic phase was washed several times with water. The aqueous phases were combined and extracted again with dichloromethane. The organic phase was dried on magnesium sulfate, filtered and the solvent was evaporated. The residue obtained (6.44 g) was purified by chromatography on silica gel (eluent:dichloromethane followed by dichloromethane/methanol 99:1) to yield the desired compound as a white solid.

Yield: 48%

Rf (dichloromethane/methanol 98:2): 0.70

IR: νNH 3400 cm⁻¹; νCO ester 1716 cm⁻¹; νCO carbamate 1689 cm⁻¹

MP: 104-111 ° C.

NMR (¹H, CDCl₃): 1.42 (s, 18H, CH₃ (BOC)); 2.46 (s, 3H, CH₃); 3.22 and 3.41 (multiplet, 4H, BOCNH—CH₂—CH—CH₂—NHBOC); 4.56 (m, 1H, BOCNH—CH₂—CH—CH₂—NHBOC); 5.04-5.11 (multiplet, 2H, —NHBOC); 7.36 (d, 2H, aromatics, J=8.5 Hz); 7.36 (d, 2H, aromatics, J=8.5 Hz).

MS (MALDI-TOF): M+23=467 (M+Na⁺); M+39=483 (M+K⁺)

### Preparation of 1,3-di(tert-butyloxycarbonylamino)-2-acetylthiopropane (example 15b)

1,3-(ditert-butoxycarbonylamino)-2-(p-toluenesulfonyloxy)propane (example 15a) (0.500 g, 1.12 mmol) and potassium thioacetate (0.161 g, 1.41 mmol) were dissolved in acetone and the medium was refluxed for 48 hours. One equivalent of potassium thioacetate was added after 24 hours of reflux. The reaction was brought to room temperature and the solvent evaporated. The residue was taken up in diethyl ether and filtered on Celite®. The filtrate was evaporated. The product obtained (0.48 g) was purified by chromatography on silica gel (eluent:dichloromethane/ethyl acetate 98:2) to give the desired compound as an ochre solid.

Yield: 84%

Rf (dichloromethane/methanol 98:2): 0.45

IR: νNH 3350 cm⁻¹; νCO ester 1719 cm⁻¹; νCO carbamate 1691 cm⁻¹

MP: 93-96° C.

NMR (¹H, CDCl₃): 1.45 (s, 18H, CH₃ (BOC)); 2.34 (s, 3H, CH₃); 3.23-3.32 (m, 2H, BOCNH—CHₐH_{b}—CH—CHₐH_{b}—NHBOC); 3.38-3.43 (m, 2H, BOCNH—CHₐH_{b}—CH—CHₐH_{b}—NHBOC); 3.58-3.66 (m, 1H, BOCNH—CH₂—CH—CH₂—NHBOC); 5.22 (multiplet, 2H, —NHBOC).

MS (MALDI-TOF): M+23=371 (M+Na⁺)

### Preparation of 1,3-di(tert-butyloxycarbonylamino)-2-mercaptopropane (example 15c)

1,3-di(tert-butoxycarbonylamino)-2-(acetylthio)propane (example 15b) (0.380 g, 1.2 mmol) diluted in methanol (10 ml) was added to a 20% potassium carbonate solution in methanol (2.14 ml, 12.4 mmol), deoxygenated under a stream of nitrogen. The reaction mixture was stirred under nitrogen at room temperature for 20 hours, then acidified to pH 6 with acetic acid. The solvents were vacuum evaporated. The residue was taken up in water and extracted with chloroform. The organic phases were combined, dried on magnesium sulfate, then filtered and dried to give the desired product in the form of a white solid which was promptly used in the next reaction.

Yield: 90%

Rf (dichloromethane/methanol 98:2): 0.56

IR: νNH 3370 cm⁻¹; νCO carbamate 1680 cm⁻¹

NMR (¹H, CDCl₃): 1.46 (s, 18H, CH₃ (BOC)); 2.98-3.12 (multiplet, 3H, BOCNH—CHₐH_{b}—CH—CHₐH_{b}—NHBOC and BOCNH—CH₂—CH—CH₂—NHBOC); 3.46-3.50 (m, 2H, BOCNH—CHₐH_{b}—CH—CHₐH_{b}—NHBOC); 5.27 (multiplet, 2H, —NHBOC).

### Preparation of 1,3-di(tert-butyloxycarbonylamino)-2-(tetradecylthioacetylthio) propane (example 15d)

1,3-[di(tert-butoxycarbonylamino)]-2-mercaptopropane (example 15c) (0.295 g, 0.963 mmol) was dissolved in dichloromethane (40 ml). Dicyclohexylcarbodiimide (0.199 g, 0.963 mmol), dimethylaminopyridine (0.118 g, 0.963 mmol) and tetradecylthioacetic acid (example 1) (0.278 g, 0.963 mmol) were then added. The reaction mixture was stirred at room temperature and the progress of the reaction was monitored by thin-layer chromatography. After 20 hours of reaction, the dicyclohexylurea precipitate was filtered, washed with dichloromethane and the filtrate was evaporated. The residue obtained (0.73 g) was purified by chromatography on silica gel (eluent:dichloromethane) to give the desired comopund in the form of a white powder.

Yield: 72%

Rf (dichloromethane/ethyl acetate 95:5): 0.29

IR: νNH 3328 cm⁻¹; νCO thioester 1717 cm⁻¹; νCO carbamate 1687 cm⁻¹

MP: 47-51° C.

NMR (¹H, CDCl₃): 0.88 (t, 9H, CH₃, J=6.1 Hz); 1.26 (multiplet, 22H, —CH₂—); 1.44 (s, 18H, CH₃ (BOC)); 1.53-1.65 (m, 2H, —CH₂—CH₂—S—CH₂—CO); 2.59 (t, 2H, —CH₂—CH₂—S—CH₂—COS—, J=7.8 Hz); 3.21-3.30 (m, 2H, BOCNH—CHₐH_{b}—CH—CHₐH_{b}—NHBOC); 3.40 (s, 2H, CH₂—S—CH₂—COS—); 3.42-3.49 (m, 2H, BOCNH—CHₐH_{b}—CH—CHₐH_{b}—NHBOC); 3.62-3.65 (m, 1H, BOCNH—CH₂—CH—CH₂—NHBOC); 5.24 (multiplet, 2H, —NHBOC).

MS (MALDI-TOF): M+23=599 (M+Na⁺); M+39=615 (M+K⁺)

### Preparation of 1,3-diamino-2-(tetradecylthioacetylthio)propane dihydrochloride (example 15)

1,3-[di(tert-butoxycarbonylamino)]-2-tetradecylthioacetylthiopropane (example 15d) (0.300 g, 0.52 mmol) was dissolved in ether saturated in gaseous hydrochloric acid (55 ml). The mixture was stirred at room temperature. After 96 hours of reaction, the precipitate which formed was filtered, washed several times with diethyl ether and dried to give the desired compound as the dihydrochloride (white powder).

Yield: 59%

Rf (dichloromethane/methanol 9:1): 0.11

IR: νNH.HCl 2700-3250 cm⁻¹; νCO thioester 1701 cm⁻¹

MP: 181° C. (decomposition)

NMR (¹H, CDCl₃): 0.86 (t, 3H, CH₃, J=6 Hz); 1.24 (multiplet, 22H, —CH₂—); 1.49-1.54 (m, 2H, —CH₂—CH₂—S—CH₂—CO); 2.59 (m, 2H, —CH₂—CH₂—S—CH₂—COS-); 2.80-2.84 (m, 1H, BOCNH—CHₐH_{b}—CH—CHₐH_{b}—NHBOC); 3.03-3.09 (m, 1H, BOCNH—CHₐH_{b}—CH—CHₐH_{b}—NHBOC); 3.14 (s, 2H, CH₂—S—CH₂—COS—); 3.27-3.38 (m, 2H, BOCNH—CHₐH_{b}—CH—CHₐH_{b}—NHBOC); 3.86-3.90 (m, 1H, BOCNH—CH₂—CH—CH₂—NHBOC); 8.21 and 8.52 (2m, 2H+4H, NH₂.HCl).

### Example 16

### Preparation of 1,3-ditetradecylthioacetylamino-2-(tetradecylthioacetylthio)propane

1,3-diamino-2-tetradecylthioacetylthiopropane dihydrochloride (example 15) (100 mg, 0.225 mmol) and tetradecylthioacetic acid (example 1) (130 mg, 0.450 mmol) were dissolved in dichloromethane (30 ml) at 0° C. followed by the addition of triethylamine (68 μl), dicyclohexylcarbodiimide (139 mg, 0.675 mmol) and hydroxybenzotriazole (61 mg, 0.450 mmol). The reaction mixture was stirred at 0° C. for 1 hour then brought to room temperature for 48 hours. The dicyclohexylurea precipitate was filtered and washed with dichloromethane and the filtrate was evaporated. The residue obtained (430 mg) was purified by chromatography on silica gel (eluent:dichloromethane/ethyl acetate 95:5) to give the desired compound in the form of a white powder.

Yield: 82%

Rf (dichloromethane/methanol 98:2): 0.54

IR: νCO thioester 1660 cm⁻¹; νCO amide 1651 cm⁻¹

MP: 83-85° C.

NMR (¹H, CDCl₃): 0.89 (t, 9H, CH₃, J=6.6 Hz); 1.26 (multiplet, 66H, —CH₂—); 1.56-1.62 (multiplet, 6H, —CH₂—CH₂—S—CH₂—CO); 2.56 (t, 4H, —CH₂—CH₂—S—CH₂—CONH—, J=7.5 Hz); 2.61 (t, 2H, —CH₂—CH₂—S—CH₂—COS—, J=7 Hz); 3.26 (s, 4H, CH₂—S—CH₂—CONH—); 3.42 (s, 2H, CH₂—S—CH₂—COS—); 3.44-3.49 (m, 2H, —CONH—CHₐH_{b}—CH—CHₐH_{b}—NH—CO); 3.55-3.61 (m, 2H, —CONH—CHₐH_{b}—CH—CHₐH_{b}—NHCO—); 3.70-3.71 (m, 1H, BOCNH—CH₂—CH—CH₂—NHBOC); 7.58-7.62 (m, 2H, NHCO).

MS (MALDI-TOF): M+1=917 (M+H⁺); M+23=939 (M+Na⁺)

### Example 17

### Preparation of 1-amino-2,3-di(tetradecylthioacetylthio)propane hydrochloride

Preparation of 1-(tert-butyloxycarbonylamino)propane-2,3-diol (example 17a)

1-aminopropane-2,3-diol (5 g, 55 mmol) was dissolved in methanol (200 ml) followed by dropwise addition of triethylamine (0.5 ml per mmol of amine) and di-tert-butyl dicarbonate [(BOC)2O] wherein BOC corresponds to tertbutyloxycarbonyl (17.97 g, 82 mmol). The reaction medium was heated at 40-50° C. for 20 min then stirred at room temperature for 1 hour. After evaporation of the solvent, the colorless oily residue was purified by chromatography on silica gel (eluent:dichloromethane/methanol 95:5) to give the desired compound in the form of a colorless oil which crystallized slowly.

Yield: 99%

Rf (dichloromethane/methanol 9:1): 0.39

IR: νNH 3350 cm⁻¹; νCO ester 1746 cm⁻¹; νCO amide 1682 cm⁻¹

MP<15° C.

NMR (¹H, CDCl₃): 1.44 (s, 9H, CH₃ (BOC)); 3.16-3.31 (m, 2H, BOCNH—CH₂—CH—CH₂—OH); 3.44 (multiplet, 2H, OH); 3.16-3.31 (m, 2H, BOCNH—CH₂—CH—CH₂—OH); 3.71-3.78 (m, 1H, BOCNH—CH₂—CH—CH₂—OH); 5.24 (m, 1H, —NHBOC).

MS (MALDI-TOF): M+23=214 (M+Na⁺)

### Preparation of 1-(tert-butyloxycarbonylamino)-2,3-di(p-toluenesulfonyloxy) propane (example 17b)

This compound was synthesized according to the method described hereinabove (example 15a) from 1-(tert-butyloxycarbonylamino)-propane-2,3-diol (example 17a) and p-toluenesulfonyl chloride. The reaction produced a white powder.

Yield: 45%

Rf (dichloromethane/methanol 98:2): 0.49

IR: νNH 3430 cm⁻¹; νCO ester and carbamate 1709 cm⁻¹

MP: 112-116° C.

NMR (¹H, CDCl₃): 1.40 (s, 9H, CH₃ (BOC)); 2.46 (s, 6H, CH₃); 3.26-3.45 (m, 2H, BOCNH—CH₂—CH—CH₂—OTs); 4.04-4.14 (m, 2H, BOCNH—CH₂—CH—CH₂—OTs); 4.68 (m, 1H, BOCNH—CH₂—CH—CH₂—OTs); 4.71 (s, 1H, —NHBOC); 7.34 (d, 4H, aromatics, J=8.5 Hz); 7.69 (d, 2H, aromatics, J=8.1 Hz); 7.76 (d, 2H, aromatics, J=8.1 Hz).

MS (MALDI-TOF): M+23=522 (M+Na⁺); M+39=538 (M+K⁺)

### Preparation of 1-(tert-butyloxycarbonylamino)-2,3-di(acetylthio)propane (example 17c)

This compound was synthesized according to the method described hereinabove (example 15b) from 1-(tert-butyloxycarbonylamino)-2,3-di(p-toluenesulfonyloxy)-propane (example 17b) and potassium thioacetate. The reaction produced a white solid.

Yield: 59%

Rf (dichloromethane/ethyl acetate 95:5): 0.55

IR: νNH 3430 cm⁻¹; νCO thioester 1718 cm⁻¹; νCO carbamate 1690 cm⁻¹

MP: 62-63° C.

NMR (¹H, CDCl₃): 1.45 (s, 9H, CH₃ (BOC)); 2.35 (s, 3H, CH₃); 2.37 (s, 3H, CH₃); 3.12-3.38 (multiplet, 4H, BOCNH—CH₂—CH—CH₂—SCO—); 3.69-3.78 (m, 1H, BOCNH—CH₂—CH—CH₂—SCO—); 5.02 (s, 1H, —NHBOC).

MS (MALDI-TOF): M+23=330 (M+Na⁺)

### Preparation of 1-(tert-butyloxycarbonylamino)-2,3-dimercaptopropane (example 17d)

This compound was synthesized according to the method described hereinabove (example 1 5c) by saponification of 1-(tert-butyloxycarbonylamino)-2,3-di(acetylthio)-propane (example 17c). The reaction produced a white solid which was promptly used in the next reaction.

Yield: 95%

Rf (dichloromethane/ethyl acetate 95:5): 0.45

IR: νNH 3368 cm⁻¹; νCO carbamate 1688 cm⁻¹

MP: 62-63° C.

NMR (¹H, CDCl₃): 1.46 (s, 9H, CH₃ (BOC)); 3.04-3.11 (m, 1H, BOCNH—CH₂—CHSH—CH₂—SH); 3.26-3.35 (m, 2H, BOCNH—CH₂—CHSH—CH₂—SH); 3.43-3.52 (m, 2H, BOCNH—CH₂—CH—CH₂—SH); 4.91 (m, 2H, SH); 5.08 (s, 1H, —NHBOC).

### Preparation of 1-(tert-butyloxycarbonylamino)-2,3-di(tetradecylthioacetylthio) propane (example 17e)

This compound was synthesized according to the method described hereinabove (example 1 5d) from 1-(tert-butyloxycarbonylamino)-2,3-dimercaptopropane (example 17d) and tetradecylthioacetic acid (example 1). The reaction produced a white solid.

Yield: 50%

Rf (dichloromethane): 0.38

IR: νNH 3421 cm⁻¹; νCO thioester 1721 cm⁻¹; νCO carbamate 1683 cm⁻¹

MP: 60-62° C.

NMR (¹H, CDCl₃): 0.87 (t, 6H, CH₃, J=6.3 Hz); 1.26 (multiplet, 44H, —CH₂—); 1.45 (s, 9H, CH₃ (BOC)); 1.57-1.62 (m, 4H, —CH₂—CH₂—S—CH₂—COS—); 2.60 (t, 4H, —CH₂—CH₂—S—CH₂—COS—, J=6.9 Hz); 3.17-3.29 (m, 2H, BOCNH—CHₐH_{b}—CH—CHₐH_{b}—NHBOC); 3.29-3.38 (m, 2H, BOCNH—CHₐH_{b}—CH—CHₐH_{b}—NHBOC); 3.41 (s, 2H, CH₂—S—CH₂—COS—); 3.43 (s, 2H, CH₂—S—CH₂—COS—); 3.76-3.80 (m, 1H, BOCNH—CH₂—CH—CH₂—NHBOC); 5.03 (s, 1H, —NHBOC).

MS (MALDI-TOF): M+23=786 (M+Na⁺)

### Preparation of 1-amino-2,3-di(tetradecylthioacetylthio)propane hydrochloride (example 17)

This compound was synthesized according to the method described hereinabove (example 15) from 1-(tert-butyloxycarbonylamino)-2,3-ditetradecylthioacetyl-thiopropane (example 17e). The product was obtained as the hydrochloride (white solid).

Yield: 43%

Rf (dichloromethane): 0.19

IR: νNH.HCl 2700-3250 cm⁻¹; νCO thioester 1701 and 1676 cm⁻¹

MP: 117-128° C.

NMR (¹H, CDCl₃): 0.86 (t, 6H, CH₃, J=6 Hz); 1.24 (multiplet, 44H, —CH₂); 1.51 (m, 4H, —CH₂—CH₂—S—CH₂—COS—); 2.61 (m, 4H, —CH₂—CH₂—S—CH₂—COS—); 2.93-3.04 (m, 2H, S—CHₐH_{b}—CH—CHₐH_{b}—NH₂.HCl); 3.11-3.20 (m, 2H, S—CHₐH_{b}—CH—CHₐH_{b}—NH₂.HCl); 3.59-3.63 (multiplet, 4H, CH₂—S—CH₂—COS—); 3.72-3.84 (m, 1H, S—CH₂—CH—CH₂—NH₂.HCl); 8.12 (m, 3H, NH₂.HCl).

### Example 18

### Preparation of 1-tetradecylthioacetylamino-2,3-di(tetradecylthioacetylthio)propane

1-amino-2,3-ditetradecylthioacetylthiopropane hydrochloride (example 17) (100 mg, 0.140 mmol) and tetradecylthioacetic acid (example 1) (62 mg, 0.210 mmol) were dissolved in dichloromethane (40 ml) at 0° C. followed by the addition of triethylamine (43 ml), dicyclohexylcarbodiimide (59 mg, 0.28 mmol) and hydroxybenzotriazole (29 mg, 0.210 mmol). The reaction mixture was stirred at 0° C. for 1 hour then brought to room temperature for 24 hours. It was then heated under gentle reflux for 48 hours, then dried. The residue obtained 310 mg) was purified by chromatography on silica gel (eluent:dichloromethane/cyclohexane 8:2) and produced the desired compound as a white powder.

Yield: 96%

Rf (dichloromethane): 0.20

IR: νNH amide 3306 cm⁻¹; νCO thioester 1674 cm⁻¹; νCO amide 1648 cm⁻¹

MP: 78-80° C.

NMR (¹H, CDCl₃): 0.89 (t, 9H, CH₃, J=6.6 Hz); 1.26 (multiplet, 66H, —CH₂); 1.58-1.62 (multiplet, 6H, —CH₂—CH₂—S—CH₂—COS—); 2.56 (t, 4H, —CH₂—CH₂—S—CH₂—COS—, J=7.5 Hz); 2.61 (t, 2H, —CH₂—CH₂—S—CH₂—CONH—, J=7 Hz); 3.26 (s, 4H, CH₂—S—CH₂—COS—); 3.42 (s, 2H, CH₂—S—CH₂—CONH—); 3.44-3.49 (m, 2H, S—CHₐH_{b}—CH—CHₐH_{b}—NHCO—); 3.55-3.61 (m, 2H, —S—CHₐH_{b}—CH—CHₐH_{b}—NHCO—); 3.70-3.71 (m, 1H, —S—CH₂—CH—CH₂—NHCO—); 7.58-7.62 (m, 1H, NHCO).

MS (MALDI-TOF): M+1=934 (M+H⁺); M+23=956 (M+Na⁺); M+39=972 (M+K⁺)

### Example 19

### Preparation of 1-tetradecylthioacetylthio-2,3-di(tetradecylthioacetylamino)propane

### Preparation of 2,3-di(tetradecylthioacetylamino)-1-iodopropane (example 19a)

2,3-ditetradecylthioacetylaminopropan-1-ol (example 13) (0.200 g, 0.317 mmol) was dissolved in toluene (30 ml). Imidazole (0.054 g, 0.792 mmol), triphenylphosphine (0.208 g, 0.792 mmol) and iodine (0.161 g, 0.634 mmol) were then added in that order and the reaction was heated at 75-80° C. with stirring. After 6 hours of reaction, the solvent was evaporated and the residual product was used without further purification.

Rf (dichloromethane/methanol 98:2): 0.55

### Preparation of 2,3-di(tetradecylthioacetylamino)-1-mercaptopropane (example 19b)

Sodium hydrogen sulfide (0.089 g, 1.59 mmol) was added to 2,3-ditetradecylthioacetylamino-1-iodopropane (example 19a) (0.235 g, 0.32 mmol) dissolved in acetone (80 ml). The reaction medium was heated at 70° C. for 16 hours. The solvent was evaporated and the residue taken up in water and extracted with chloroform. The aqueous phase was acidified to pH 6 with acetic acid, then extracted again with chloroform. The organic phases were dried on magnesium sulfate and filtered and the solvent was evaporated. The residue obtained was used without further purification.

### Preparation of 1-tetradecylthioacetylthio-2,3-di(tetradecylthioacetylamino) propane (example 19)

2,3-ditetradecylthioacetylamino-1-mercaptopropane (example 19b) (0.205 g, 0.32 mmol) was dissolved in tetrahydrofuran (50 ml). Dicyclohexylcarbodiimide (98 mg, 0.47 mmol), dimethylaminopyridine (58 mg, 0.47 mmol) and tetradecylthioacetic acid (example 1) (137 mg, 0.47 mmol) were then added. The mixture was stirred at room temperature for 20 hours. The dicyclohexylurea precipitate was filtered, washed with tetrahydrofuran and the filtrate was evaporated. The residue obtained (1.14 g) was purified by chromatography on silica gel (eluent:dichloromethane) to give the desired compound in the form of an ochre powder.

Yield: 10%

Rf (dichloromethane/ethyl acetate 98:2): 0.19

IR: νCO thioester 1711-1745 cm⁻¹; νCO amide 1651 cm⁻¹

MP: 48.8-49.8° C.

NMR (¹H, CDCl₃): 0.89 (t, 9H, CH₃, J=6.3 Hz); 1.26 (multiplet, 66H, —CH₂); 1.58 (m, 6H, —CH₂—CH₂—S—CH₂—COS—); 2.46-55 (m, 4H, —CH₂—CH₂—S—CH₂—CONH); 2.65 (t, 2H, —CH₂—CH₂—S—CH₂—COS—, J=7.4 Hz); 3.24 (s, 2H, CH₂—S—CH₂—CONH—); 3.26 (s, 2H, CH₂—S—CH₂—CONH—); 3.66 (t, 2H, —COS—CH₂—CH—CH₂—NHCO); 3.79 (t, 2H, CH₂—S—CH₂—COS—, J=6.3 Hz); 4.31-4.41 (m, 2H, —COS—CH₂—CH—CH₂—NHCO); 5.00-5.05 (m, 1H, —COS—CH₂—CH—CH₂—NHCO); 7.33 (sl, 1H, NHCO); 9.27 (d, 1H, NHCO, J=8.6 Hz).

MS (MALDI-TOF): M+1=917 (M+H⁺); M+23=939 (M+Na⁺); M+39=955 (M+K⁺)

### Example 20

### Preparation of 3-tetradecylthioacetylamino-2-tetradecylthioacetylthiopropan-1-ol

### Preparation of 3-tetradecylthioacetylamino-1-triphenylmethyloxypropan-2-ol (example 20a)

Chlorotriphenylmethane (2.833 g, 10.16 mmol) was added to a solution of 3-tetradecylthioacetylaminopropane-1,2-diol (example 2) (3 g, 8.30 mmol) in pyridine (2.5 ml). The reaction mixture was stirred at 50° C. for 24 hours and the solvent was then vacuum evaporated. The residue was taken up in water and extracted with dichloromethane. The organic phase was washed with 1N aqueous hydrochloric acid then with an aqueous solution saturated in sodium chloride. It was dried on magnesium sulfate, filtered and the solvent was evaporated. The residue obtained (6.36 g) was purified by chromatography on silica gel (eluent:dichloromethane/ethyl acetate 98:2) to give the desired compound in the form of a white powder.

Yield: 69%

Rf (dichloromethane/ethyl acetate 8:2): 0.61

IR: νNH amide 3225 cm⁻¹; νCO amide1654 cm⁻¹

MP: 62.6-65.4° C.

NMR (¹H, CDCl₃): 0.89 (t, 3H, CH₃, J=6.7 Hz); 1.26 (multiplet, 22H, —CH₂); 1.50-1.57 (m, 2H, —CH₂—CH₂—S—CH₂—CONH—); 2.48 (t, 2H, —CH₂—CH₂—S—CH₂—CONH, J=7.2 Hz); 3.01 (m, 1H, OH); 3.17 (s, 2H, CH₂—S—CH₂—CONH—); 3.19 (m, 2H, —O—CH₂—CH—CH₂—NHCO or trityl-O—CH₂—CH—CH₂—NHCO); 3.27-3.36 (m, 1H, —O—CH₂—CH—CH₂—NHCO or trityl-O—CH₂—CH—CH₂—NHCO); 3.54-3.62 (m, 1H, —O—CH₂—CH—CH₂—NHCO or trityl-O—CH₂—CH—CH₂—NHCO); 3.93 (m, 1H, —O—CH₂—CH—CH₂—NHCO); 7.16 (t, 1H, NHCO, J=5.7 Hz); 7.23-7.35 (multiplet, 9H, aromatic H); 7.41-7.45 (multiplet, 6H, aromatic H).

MS (MALDI-TOF): M+23=626 (M+Na⁺)

### Preparation of 2-iodo-3-tetradecylthioacetylamino-1-triphenylmethyloxypropane (example 20b)

3-tetradecylthioacetylamino-1-triphenylmethyloxypropan-2-ol (example 20a) (2 g, 3.31 mmol) was dissolved in toluene (100 ml). Imidazole (0.564 g, 8.28 mmol), triphenylphosphine (2.171 g, 8.28 mmol) and iodine (1.681 g, 6.62 mmol) were then added in that order. The reaction medium was stirred at room temperature for 20 hours. A saturated sodium bisulfite solution was added until complete blanching of the reaction medium. The phases were separated and the aqueous phase was extracted with toluene. The organic phases were combined, washed with saturated sodium chloride solution, dried on magnesium sulfate and filtered. The residue obtained after evaporation of the solvent (4.65 g) was purified by chromatography on silica gel (eluent:dichlromethane) to give the desired compound in the form of a yellow oil.

Yield: 21%

Rf (dichloromethane/ethyl acetate 95:5): 0.58

IR: νCO amide 1668 cm⁻¹; νCH arom. monosubstituted 748 and 698 cm⁻¹

NMR (¹H, CDCl₃): 0.89 (t, 3H, CH₃, J=6.5 Hz); 1.26 (multiplet, 20H, —CH₂); 1.53-1.63 (m, 2H, —CH₂—CH₂—CH₂—S—CH₂—CONH—); 2.63 (m, 2H, —CH₂—CH₂—CH₂—S—CH₂—CONH); 3.13-3.30 (m, 2H, —CH₂—CH₂—S—CH₂—CONH); 3.34 (s, 2H, CH₂—S—CH₂—CONH—); 3.67-3.71 (m, 2H, —O—CH₂—CH—CH₂—NHCO or trityl-O—CH₂—CH—CH₂—NHCO); 3.88-3.94 (m, 2H, —O—CH₂—CH—CH₂—NHCO or trityl-O—CH₂—CH—CH₂—NHCO); 4.76 (m, 1H, —O—CH₂—CH—CH₂—NHCO); 7.25-7.36 (multiplet, 9H, aromatic H); 7.45-7.49 (multiplet, 6H, aromatic H).

MS (MALDI-TOF): M-127=586 (M−I)

### Preparation of 2-mercapto-3-tetradecylthioacetylamino-1-triphenylmethyloxypropane (example 20c)

Sodium hydrogen sulfate hydrate (38 mg, 0.68 mmol) was prepared as a suspension in ethanol (20 ml) followed by the addition of 2-iodo-3-tetradecylthioacetylamino-1-triphenylmethyloxypropane (example 20b) (200 mg, 0.28 mmol). The reaction medium was heated at 70° C. 238 mg of sodium hydrogen sulfate hydrate were added over several days. After 6.5 days, the solvent was evaporated and the residue taken up in dichloromethane and washed with water. The aqueous phase was re-extracted and the combined organic phases were washed with 0.5 N hydrochloric acid then with saturated sodium chloride solution, then dried on magnesium sulfate. The salt was filtered and the solvent evaporated. The residue obtained was used without further purification.

Rf (dichloromethane/ethyl acetate 95:5): 0.33

### Preparation of 3-tetradecylthioacetylamino-2-tetradecylthioacetylthio-1-triphenylmethyloxy-propane (example 20d)

2-mercapto-3-tetradecylthioacetylamino-1-triphenylmethyloxypropane (example 20c) (174 mg, 0.28 mmol) was dissolved in tetrahydrofuran (20 ml). Dicyclohexylcarbodiimide (88 mg, 0.42 mmol), dimethylaminopyridine (51 mg, 0.42 mmol) and tetradecylthioacetic acid (121 mg, 0.42 mmol) were then added and the reaction medium was stirred at room temperature. After 20 hours of reaction, the solvent was evaporated and the residue obtained (450 mg) was purified by flash chromatography (eluent:dichloromethane/cyclohexane 3:7 to 5-5) to give the desired compound in the form of a white powder.

Yield: 76%

Rf (dichloromethane): 0.39

IR: νCO thioester and amide 1745 to 1640 cm⁻¹

MP: 48.5-51.9° C.

NMR (¹H, CDCl₃): 0.89 (t, 6H, CH₃, J=6.3 Hz); 1.26 (multiplet, 44H, —CH₂); 1.62 (m, 4H, —CH₂—CH₂—S—CH₂—CO—); 2.42 (t, 2H, —CH₂—CH₂—S—CH₂—CONH—, J=7.5 Hz); 2.68 (t, 2H, —CH₂—CH₂—S—CH₂—COS—, J=7.5 Hz); 3.14 (s, 2H, CH₂—S—CH₂—CONH—); 3.25 (s, 2H, CH₂—S—CH₂—COS—); 3.50-3.59 (m, 1H, —O—CH₂—CH—CH₂—NHCO or trityl-O—CH₂—CH—CH₂—NHCO); 3.66-3.72 (m, 1H, —O—CH₂—CH—CH₂—NHCO or trityl-O—CH₂—CH—CH₂—NHCO); 3.96 (m, 1H, —O—CH₂—CH—CH₂—NHCO or trityl-O—CH₂—CH—CH₂—NHCO); 3.54-3.62 (m, 1H, —O—CH₂—CH—CH₂—NHCO or trityl-O—CH₂—CH—CH₂—NHCO); 5.16 (m, 1H, —O—CH₂—CH—CH₂—NHCO); 7.04 (m, 1H, NHCO, J=5.7 Hz); 7.25-7.34 (multiplet, 9H, aromatic H); 7.42-7.45 (multiplet, 9H, aromatic H).

MS (MALDI-TOF): M+23=889 (M+Na⁺)

### Preparation of 3-tetradecylthioacetylamino-2-tetradecylthioacetylthiopropan-1-ol (example 20)

3-tetradecylthioacetylamino-2-tetradecylthioacetylthio-1-triphenylmethyloxy-propane (example 20d) (187 mg, 0.21 mmol) was dissolved in ether saturated with gaseous hydrochloric acid (12 ml). The reaction medium was stirred at room temperature for 20 hours. The precipitate which-formed was filtered and washed with diethyl ether to give the desired compound in the form of a white powder.

Yield: 52%

Rf (dichloromethane/methanol 98:2): 0.48

IR: νCO thioester 1704cm⁻¹; νCO amide 1646 cm⁻¹

MP: 88.4-94.1° C.

NMR (¹H, CDCl₃): 0.89 (t, 6H, CH₃, J=6.4 Hz); 1.26-1.37 (multiplet, 44H, —CH₂); 1.55-1.61 (m, 4H, —CH₂—CH₂—S—CH₂—CO—); 2.55 (t, 2H, —CH₂—CH₂—S—CH₂—CONH—, J=7 Hz); 2.65 (t, 2H, —CH₂—CH₂—S—CH₂—COS—, J=7 Hz); 3.26 (s, 2H, CH₂—S—CH₂—CONH—); 3.27 (s, 2H, CH₂—S—CH₂—COS—); 3.36-3.38 (m, 1H, —O—CH₂—CH—CH₂—NHCO); 3.58-3.64 (m, 1H, —O—CH₂—CH—CH₂—NHCO); 4.02 (m, 1H, —O—CH₂—CH—CH₂—NHCO); 4.11-4.25 (m, 2H, HO—CH₂—CH—CH₂—NHCO); 7.34 (m, 1H, NHCO).

MS (MALDI-TOF): M+23=670 (M+Na⁺)

### Example 21

### Preparation of 3-tetradecylthioacetylamino-1-tetradecylthiacetyloxy-2-tetradecylthioacetylthiopropane

3-tetradecylthioacetylamino-2-tetradecylthioacetylthiopropan-1-ol (example 20) (64 mg, 0.10 mmol) was dissolved in tetrahydrofuran (7 ml). Dicyclohexylcarbodiimide (31 mg, 0.15 mmol), dimethylaminopyridine (18 mg, 0.15 mmol) and tetradecylthioacetic acid (example 1) (43 mg, 0.15 mmol) were then added. The mixture was stirred at room temperature for 20 hours. The dicyclohexylurea precipitate was filtered and the filtrate was evaporated. The residue obtained (140 mg) was purified by flash chromatography (eluent:dichloromethane) to give the desired compound in the form of a white powder.

Yield: 17%

Rf (dichloromethane/ethyl acetate 98:2): 0.23

IR: νCO ester 1730 cm⁻¹; νCO thioester 1671 cm⁻¹; νCO amide 1645 cm⁻¹

MP: 59.0-63.4° C.

NMR (¹H, CDCl₃): 0.89 (t, 9H, CH₃, J=6.5 Hz); 1.26-1.37 (multiplet, 66H, —CH₂); 1.58-1.63 (m, 6H, —CH₂—CH₂—S—CH₂—CO—); 2.53 (t, 2H, —CH₂—CH₂—S—CH₂—CONH—, J=7.6 Hz); 2.61-2.67 (m, 4H, —CH₂—CH₂—S—CH₂—COS— and —CH₂—CH₂—S—CH₂—COO— ); 3.23 (s, 4H, CH₂—S—CH₂—CONH— and CH₂—S—CH₂—COO—); 3.24 (s, 2H, CH₂—S—CH₂—COS—); 3.50-3.57 (m, 1H, —O—CH₂—CH—CH₂—NHCO); 3.63-3.72 (m, 1H, —O—CH₂—CH—CH₂—NHCO); 4.19-4.25 (m, 1H, —O—CH₂—CH—CH₂—OCO); 3.63-3.72 (m, 1H, —O—CH₂—CH—CH₂—OCO); 5.19 (m, 1H, —O—CH₂—CH—CH₂—NHCO); 7.20 (m, 1H, NHCO).

MS (MALDI-TOF): M+23=940 (M+Na⁺)

### Example 22

### Preparation of 3-amino-2-tetradecylthioacetyloxy-1-tetradecylthioacetylthiopropane hydrochloride

### Preparation of 1-tert-butyloxycarbonylamino-3-iodopropane-2-ol (example 22a)

1-[(tert-butyloxycarbonyl)amino]propane-2,3-diol (example 17a) (3.88 g, 20 mmol) was dissolved in toluene (250 ml). Imidazole (1.73 g, 25 mmol), triphenylphosphine (6.65 g, 25 mmol) and iodine (5.15 g, 20 mmol) were then added in that order. The reaction medium was stirred at room temperature for 17 hours and 0.5 equivalents of imidazole, triphenylphosphine and iodine were added. After 21 hours of reaction, a saturated sodium sulfite solution was added until complete blanching of the reaction medium. The phases were allowed to settle and the aqueous phase was extracted twice with toluene. The combined organic phases were washed with saturated sodium chloride solution, dried on magnesium sulfate, filtered and the solvent evaporated. The residue obtained (11.02 g) was purified by chromatography on silica gel (eluent:dichloromethane/ethyl acetate 95:5) to give the desired compound as a yellow paste which was promptly used in the next reaction.

Yield: 41%

Rf (dichloromethane/methanol 98:2): 0.24

IR: νNH amide 3387 cm⁻¹; νCO carbamate 1678 cm⁻¹

### Preparation of 3-acetylthio-1-tert-butyloxycarbonylaminopropan-2-ol (example 22b)

1-(tert-butyloxycarbonylamino)-3-iodopropan-2-ol (example 22a) (2 g, 6.64 mmol) and potassium thioacetate (0.948 g, 8.30 mmol) were dissolved in acetone (30 ml) and the medium was refluxed for 16 hours. The solvent was vacuum evaporated and the residue was taken up in diethyl ether, then filtered on Celite®. The filtrate was evaporated. The residue obtained (1.69 g) was purified by chromatography on silica gel (eluent:dichloromethane/ethyl acetate 98:2) then repurified by flash chromatography (eluent:dichloromethane) to give the desired compound in the form of a yellow oil.

Yield: 27%

Rf (dichloromethane/ethyl acetate 95:5): 0.31

IR: νNH amide 3367 cm⁻¹; νCO thioester 1744 cm⁻¹; νCO carbamate 1697 cm⁻¹

NMR (¹H, CDCl₃): 1.26 (m, 9H, CH₃ (boc)); 2.37 (s, 3H, COCH₃); 3.04 (m, 1H, —NH—CH₂—CH—CH₂—S— or —NHCH₂—CH—CH₂—S—); 3.24 (m, 1H, —NH—CH₂—CH—CH₂—S— or —NHCH₂—CH—CH₂—S—); 3.30-3.41 (m, 2H, —NH—CH₂—CH—CH₂—S— or —NHCH₂—CH—CH₂—S—); 4.86 (sl, 1H, OH); 4.96 (m, 1H, —NH—CH₂—CH—CH₂—S—).

### Preparation of 1-tert-butyloxycarbonylamino-3-mercaptopropan-2-ol (example 22c)

3-acetylthio-1-tert-butyloxycarbonylaminopropan-2-ol (example 22b) (0.307 g, 1.23 mmol) diluted in a minimum of methanol (7 ml) was added to a 20% potassium carbonate solution (3.49 ml, 12.31 mmol) in methanol, deoxygenated under a stream of nitrogen. The medium was stirred at room temperature under a stream of nitrogen for 20 hours, then acidified to pH 6 with acetic acid and concentrated to dryness. The residue obtained was taken up in water and extracted with dichloromethane. The organic phase was dried on magnesium sulfate, filtered and concentrated. The oily residue obtained was used immediately in the next reaction without further purification.

Yield: 78%

Rf (dichloromethane/ethyl acetate): 0.07

### Preparation of 1-tert-butyloxycarbonylamino-2-tetradecylthioacetyloxy-3-tetradecylthioacetylthiopropane (example 22d)

1-(tert-butyloxycarbonylamino)-3-mercaptopropan-2-ol (example 22c) (0.200 g, 96 mmol) was dissolved in dichloromethane (50 ml). Dicyclohexylcarbodiimide (0.398 g, 1.93 mmol), dimethylaminopyridine (0.236 g, 1.93 mmol) and tetradecylthioacetic acid (example 1) (0.557 g, 1.93 mmol) were then added. The mixture was stirred at room temperature for 20 hours. The dicyclohexylurea precipitate was filtered, washed with dichloromethane and the filtrate was evaporated. The residue obtained (1.2 g) was purified by chromatography on silica gel (eluent:dichloromethane) to give the desired compound in the form of a white paste.

Yield: 47%

Rf (dichloromethane): 0.26

IR: νNH amide 3314 cm⁻¹; νCO ester, amide and thioester 1682 to 1744 cm⁻¹

NMR (¹H, CDCl₃): 0.89 (t, 6H, CH₃, J=6.5 Hz); 1.27 (multiplet, 40H, CH₂); 1.45 (multiplet, 9H, CH₃ (BOC)); 1.56-1.63 (m, 4H, —CH₂—CH₂—CH₂—S—CH₂—CO—); 2.65 (m, 4H, —CH₂—CH₂—S—CH₂—CO—); 2.92 (s, 4H, —CH₂—S—CH₂—CO—); 2.96 (m, 4H, —CH₂—S—CH₂—CO—); 3.24-3.40 (m, 2H, —NH—CH₂—CH—CH₂—S— or —NHCH₂—CH—CH₂—S); 3.44-3.51 (m, 2H, —NH—CH₂—CH—CH₂—S— or —NHCH₂—CH—CH₂—S—); 4.91 (m, 1H, —NH—CH₂—CH—CH₂—S—); 5.19 (m, 1H, NHCO).

MS (MALDI-TOF): M+23=770 (M+Na⁺)

### Preparation of 1-amino-2-tetradecylthioacetyloxy-3-tetradecylthioacetylthio propane hydrochloride (example 22)

1-(tert-butoxycarbonylamino)-2-tetradecylthioacetyloxy-3-tetradecylthioacetyl-thiopropane (example 22d) (300 mg, 0.40 mmol) was dissolved in diethyl ether saturated with gaseous hydrochloric acid (70 ml) and the reaction medium was stirred at room temperature for 72 hours. The precipitate which formed was filtered, washed with diethyl ether and dried to give the desired compound in the form of a white powder.

Yield: 42%

IR: νCO ester 1733 cm⁻¹; νCO thioester 1692 cm⁻¹

MP: 82° C. (decomposition)

NMR (¹H, CDCl₃): 0.86 (t, 6H, CH₃, J=6.6 Hz); 1.24 (multiplet, 44H, —CH₂); 1.52 (m, 4H, —CH₂—CH₂—S—CH₂—CO—); 2.52-2.62 (m, 4H, —CH₂—CH₂—S—CH₂—CO—); 3.07-3.15 (multiplet, 4H, —S—CH₂—CH—CH₂—NH₂); 3.40 (s, 2H, CH₂—S—CH₂—COO—); 3.61 (s, 2H, CH₂—S—CH₂—COS—); 5.12 (m, 1H, —S—CH₂—CH—CH₂-NH₂); 8.01 (m, 3H, —NH₂.HCl)

### Example 23

### Preparation of 1-tetradecylthioacetylamino-2-tetradecylthiacetyloxy-3-tetradecylthioacetylthiopropane

1-amino-2-tetradecylthioacetyloxy-3-tetradecylthioacetyl-thiopropane hydrochloride (example 22) (100 mg, 0.15 mmol) and tetradecylthioacetic acid (example 1) (63 mg, 0.22 mmol) were dissolved in dichloromethane (30 ml) at 0° C. followed by the addition of triethylamine (0.044 ml), dicyclohexylcarbodiimide (60 mg, 0.29 mmol) and hydroxybenzotriazole (30 mg, 0.22 mmol). The reaction medium was stirred at 0° C. for 1 hour then brought to room temperature for 48 hours. The dicyclohexylurea precipitate was filtered, washed with dichloromethane and the filtrate was evaporated. The residue obtained (263 mg) was purified by flash chromatography (eluent:dichloromethane/ethyl acetate 98:2) to give the desired compound in the form of a white powder.

Yield: 98%

Rf (dichloromethane/ethyl acetate 95:5): 0.38

IR: νNH amide 3340 cm⁻¹; νCO ester 1727 cm⁻¹; νCO amide and thioester 1655 and 1669 cm⁻¹

MP: 63.9-67.1° C.

NMR (¹H, CDCl₃): 0.89 (t, 9H, CH₃, J=6.2 Hz); 1.26 (multiplet, 66H, —CH₂); 1.54-1.66 (m, 6H, —CH₂—CH₂—S—CH₂—CO—); 2.52-2.67 (m, 6H, —CH₂—CH₂—S—CH₂—CO—); 3.08 (m, 1H, —S—CH₂—CH—CH₂—NHCO or —S—CH₂—CH—CH₂—NHCO); 3.21 (s, 2H, CH₂—S—CH₂—CONH—); 3.23 (s, 2H, CH₂—S—CH₂—COO—); 3.27 (m, 1H, —S—CH₂—CH—CH₂—NHCO or —S—CH₂—CH—CH₂—NHCO); 3.43 (s, 2H, CH₂—S—CH₂—COS—); 3.50 (m, 1H, —S—CH₂—CH—CH₂—NHCO or —S—CH₂—CH—CH₂—NHCO); 3.62 (m, 1H, —S—CH₂—CH—CH₂—NHCO or —S—CH₂—CH—CH₂—NHCO); 5.06 (m, 1H, —COS—CH₂—CH—CH₂—NHCO); 7.24 (t, 1H, —NHCO, J=6.7 Hz)

MS (MALDI-TOF): M+1=918 (M+H⁺); M+23=940 (M+Na⁺)

### Example 24

### Method of Preparation of the Inventive Compounds

To perform the in vitro experiments described in the following examples, the inventive compounds were prepared in the form of an emulsion as described below.

An emulsion comprising an inventive compound and phosphatidylcholine (PC) was prepared as described by Spooner et al. (Spooner, Clark et al. 1988). The inventive compound was mixed with PC in a 4:1 (m/m) ratio in chloroform, the mixture was dried under nitrogen, then vacuum evaporated overnight; the resulting powder was taken up in 0.16 M KCl containing 0.01 M EDTA and the lipid particles were then dispersed by ultrasound for 30 minutes at 37° C. The liposomes so formed were then separated by ultracentrifugation (XL 80 ultracentrifuge, Beckman Coulter, Villepinte, France) at 25,000 rpm for 45 minutes to recover liposomes having a size greater than 100 nm and close to that of chylomicrons. Liposomes composed only of PC were prepared concurrently to use as negative control.

The composition of the liposomes in the inventive compound was estimated by using the enzyme colorimetric triglyceride assay kit. The assay was carried out against a standard curve, prepared with the lipid calibrator CFAS, Ref. 759350 (Boehringer Mannheim GmbH, Germany). The standard curve covered concentrations ranging from 16 to 500 μg/ml. 100 μl of each sample dilution or calibration standard were deposited per well on a titration plate (96 wells). 200 μl of triglyceride reagents, ref. 701912 (Boehringer Mannheim GmbH, Germany) were then added to each well, and the entire plate was incubated at 37° C. for 30 minutes. Optical densities (OD) were read on a spectrophotometer at 492 nm. Triglyceride concentrations in each sample were calculated from the standard curve plotted as a linear function y=ax+b, where y represents OD and x represents triglyceride concentrations.

Liposomes containing the inventive compounds, prepared in this manner, were used for in vitro experiments described in the following examples.

### Example 25

### Evaluation of PPAR Activation in vitro

The inventive compounds which were tested are the compounds whose preparation is described in examples 2 to 23 hereinabove.

Nuclear receptors of the PPAR subfamily which are activated by two major pharmaceutical classes—fibrates and glitazones, widely used in the clinic for the treatment of dyslipidemias and diabetes—play an important role in lipid and glucose homeostasis. The following experimental data show that the inventive compounds activate PPARα in vitro.

PPAR activation was tested in vitro in RK13 fibroblast cell lines or in a hematocyte line HepG2 by measuring the transcriptional activity of chimeras composed of the DNA binding domain of the yeast gal4 transcription factor and the ligand binding domain of the different PPARs. The example below is given for HepG2 cells.

A—Culture Protocols:

HepG2 cells were from ECACC (Porton Down, UK) and were grown in DMEM medium supplemented with 10% (V/V) fetal calf serum, 100 U/ml penicillin (Gibco, Paisley, UK) and 2 mM L-glutamine (Gibco, Paisley, UK). The culture medium was changed every two days. Cells were kept at 37° C. in a humidified 95% air/5% CO₂ atmosphere.

B—Description of Plasmids used for Transfection:

The plasmids pG5TkpGL3, pRL-CMV, pGal4-hPPARα, pGal4-hPPARγ and pGal4-f have been described by Raspe et al. (Raspe, Madsen et al. 1999). The pGal4-mPPARα and pGal4-hPPARβ constructs were obtained by cloning PCR-amplified DNA fragments corresponding to the DEF domains of the mouse PPARα and human PPARα nuclear receptors, respectively, into the pGal4-f vector.

C—Transfection:

HepG2 cells were seeded in 24-well culture dishes at 5×10⁴ cells/well and transfected for 2 hours with the reporter plasmid pG5TkpGL3 (50 ng/well), the expression vectors pGal4-f, pGal4-mPPARα, pGal4-hPPARα, pGal4-hPPARγ, or pGal4-hPPARβ (100 ng/well) and the transfection efficiency control vector pRL-CMV (1 ng/well) according to the previously described protocol (Raspe, Madsen et al. 1999), then incubated for 36 hours with the test compounds. At the end of the experiment, the cells were lysed (Gibco, Paisley, UK) and luciferase activity was determined with a Dual-Luciferase™ Reporter Assay System kit (Promega, Madison, Wisc., USA) according to the supplier's instructions. The protein content of the cell extracts was then measured with the Bio-Rad Protein Assay kit (Bio-Rad, Munich, Germany) as directed by the supplier.

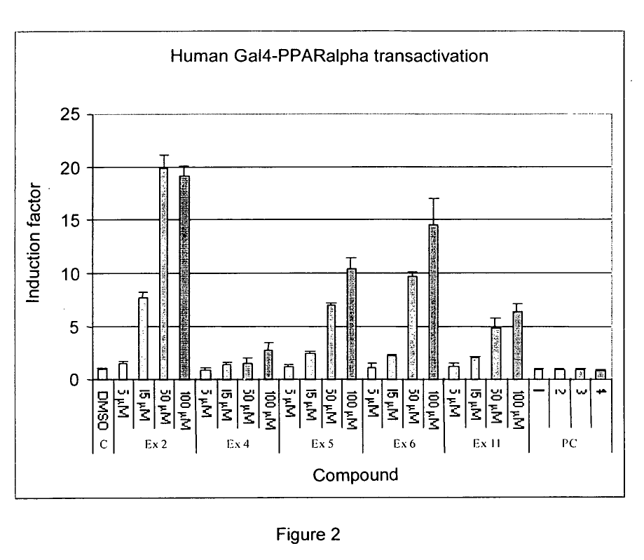

The inventors demonstrate an increase in luciferase activity in cells treated with the inventive compounds and transfected with the pGal4-hPPARα plasmid. Said induction of luciferase activity indicates that the inventive compounds are activators of PPARα. FIG. 2 gives an example of the results obtained with the inventive compounds.

FIG. 2: HepG2 cells transfected with Gal4/PPARα plasmids were incubated with different concentrations (5, 15, 50 and 100 μM) of the inventive comopunds (Ex 2, Ex 4, Ex 5, Ex 6, Ex 11) for 24 h and with different concentrations of the vehicle (PC) noted 1, 2, 3, 4 as controls for the 5, 15, 50 and 100 μM concentrations of the inventive compounds (according to the 4:1 (m/m) ratio described in example 24 (Method of preparation of the inventive compounds)). The results are expressed as the induction factor (luminescent signal of treated cells divided by luminescent signal of untreated cells) after the different treatments. The higher the induction factor the more potent the PPARα agonist activity. The results show that inventive compound Ex 2 produced a maximum 19.8-fold induction of the luminescent signal at 50 μM, 19.2 at 100 μM, 7.7 at 15 μM and 1.5 at 5 μM. Inventive compound Ex 5 also showed a dose-dependent increase in the induction factor of 10.5 at 100 μM, 7 at 50 μM, 2.5 at 15 μM and 1.2 at 5 μM. Inventive compound Ex 6 also induced an increase in the luminescent signal, revealing an activity on the PPARα nuclear receptor. The induction factors for inventive compound Ex 6 were 14.5 at 100 μM, 9.6 at 50 μM, 2.2 at 15 μM and 1.1 at 5 μM. In contrast, when the cells were incubated with the vehicle (PC liposome), no significant induction was observed.

These results demonstrate that the inventive compounds tested exhibit significant PPARα ligand activity and therefore enable the transcriptional activation thereof.

D—RNA Analysis:

Messenger RNA was extracted from HepG2 cells using the reagents in the Absolutely RNA RT-PCR miniprep kit (Stratagene, France) according to the supplier's instructions, then assayed by spectrophotometry and quantified by semi-quantitative or quantitative RT-PCR using the Light Cycler Fast Start DNA Master Sybr Green I kit (Hoffman-La Roche, Basel, Switzerland) on a Light Cycler System (Hoffman-La Roche, Basel, Switzerland). Primer pairs specific for the ACO and Apo AI genes, which are target genes of PPARα were used as probes. Primer paris specific for the 36B4, beta-actin and GAPDH genes were used as control probes (see Table 1 below).

TABLE I

| | | Semi quantitative | | Quantitative | | |
|---|---|---|---|---|---|---|
| | | PCR | | PCR | | |
| | | | No. | | No. cycles | |
| Name | Sequence | Tm | cycles | Tm | removed | Gene |
| ApoAl_r_1_s 741 | GCCTGAATCTCCT | 58° C. | 25 | 58° C. | 18 to 20 | Apo Al |
| | GGACAACTG | | | | | |
| ApoAl_r_1_as 742 | ATGCCTTTGCATCT | | | | | |
| | CCTTCG | | | | | |
| ApoB_r_1_s 743 | ATACAGCCTGAGT | 55° C. | 30 | X | X | Apo B |
| | GAGCCTCTTCAG | | | | | |
| ApoB_r_1_as 744 | CCAGGGAGTTGGA | | | | | |
| | GACCGTG | | | | | |
| GAPDH_h_1_s 390 | GACATCAAGAAGG | 55° C. | 25 | 55° C. | 20 | GAPDH |
| | TGGTGAA | | | | (variable) | |
| GAPDH_h_1_as | CCACATACCAGGA | | | | | |
| 389 | AATGAGC | | | | | |
| beta-actin_h_1_s | TTCAACTCCATCAT | 55° C. | 25 | 55° C. | variable | β actin |
| 189 | GAAGTGTGAC | | | | | |
| beta-actin_h_1_as | TCGTCATACTCCTT | | | | | |
| 188 | GCTTGCTGATCC | | | | | |
| CPT1_r_1_s 517 | GCTGGCTTATCGT | 60° C. | 25 | 60° C. | 20 to 25 | CPT-I |
| | GGTGGTG | | | | | |
| CPT1_r_1_as 516 | GACCTGAGAGGAC | | | | | |
| | CTTGACC | | | | | |
| 36B4_h_1_s 177 | CATGCTCAACATCT | X | X | 55° C. | 23 | 36B4 |
| | CCCCCTTCTCC | | | | | |
| 36B4_h_1_as 178 | GGGAAGGTGTAAT | | | | | |
| | CCGTCTCCACAG | | | | | |
| ACOX1_r_1_as | CGCATCCATTTCTC | 60° C. | 25 | 60° C. | 18 to 24 | ACO |
| 457 | CTGCTG | | | | | |
| ACOX1_r_1_s 458 | TTCTGTCGCCACCT | | | | | |
| | CCTCTG | | | | | |
| ApoCIII_r_1_s 797 | ATGCAGCCCCGAA | 55° C. | 30 | 55° C. | 28 to 30 | Apo CIII |
| | TGCTCCTCATCGT | | | | | |
| | GG | | | | | |
| ApoCIII_r_1_as | TCACGGCTCAAGA | | | | | |
| 798 | GTTGGTGTTAC | | | | | |
| CPT2_r_1_s_725 | CAGAAGCCTCTCTT | 55° C. | 25 | X | X | CPT-II |
| | GGATGACAG | | | | | |
| CPT2_r_1_as_726 | TTGGTTGCCCTGG | | | | | |
| | TAAGCTG | | | | | |
| ABCA1_h_2_s | CTGAGGTTGCTGC | 65° C. | 21 | X | X | ABCA1 |
| | TGTGGAAG | | | | | |
| ABCA1_h_2_as | CATCTGAGAACAG | | | | | |
| | GCGAGCC | | | | | |

The results obtained confirm that the compounds tested are capable of very potent activation of the PPAR α nuclear receptor (results not shown).

### Example 26

### Evaluation of the Effects on Lipid Metabolism in vivo

The inventive compounds which were tested are the compounds the preparation of which is described in examples 2 to 23 hereinabove.

Fibrates, widely used in human medicine for the treatment of dyslipidemiae involved in the development of atherosclerosis, one of the leading causes of morbidity and mortality in industrialized countries, are potent activators of the PPARα nuclear receptor. The latter regulates the expression of genes involved in the transport (apolipoproteins such as Apo AI, ApoAII and ApoC-III, membrane transporters such as FAT (Fatty Acid Transporter) or catabolism of lipids (ACO (Acyl CoA Oxidase), CPT-I or CPT-II (Carnitine Palmitoyl Transferase I and II)). Treatment of rodents with PPARα activators therefore leads to a decrease in plasma cholesterol and triglyceride levels.

The following protocols were designed to demonstrate a decrease in circulating triglyceride and cholesterol levels, and also highlight the interest of the inventive compounds for preventing and/or treating cardiovascular diseases.

1) Treatment of Animals

Sprague-Dawley rats weighing 200 to 230 g (Charles River, L'Arbresle, France) were housed in a 12-hour light/dark cycle at a constant temperature of 20±3° C. After a 1-week acclimatization period, rats were weighed and distributed into groups of 8 animals selected such that the distribution of plasma cholesterol and triglyceride levels was uniform. The test compounds were suspended in a vehicle (0.5% carboxymethylcellulose (CMC) and 0.1% Tween) and administered by intragastric gavage at the indicated doses, once a day for 15 days. Animals had access to food and water ad libitum. At the end of the experiments, animals were weighed and sacrificed under anesthesia after a 5-hour fast. Blood was collected on EDTA. Plasma was isolated by centrifugation at 3000 rpm for 20 minutes. Liver samples were removed and stored frozen in liquid nitrogen for subsequent analysis.

The carboxymethylcellulose used is a sodium salt of intermediate viscosity carboxymethylcellulose (Ref. C4888, Sigma-Aldrich, France). Tween used is Polyoxyethylenesorbitan Monooleate (Tween 80, Ref. P8074, Sigma-Aldrich, France)

2) Determination of Serum Lipids and Apolipoproteins

Lipid concentrations in plasma (total cholesterol and triglycerides) were determined by a calorimetric assay (Bio-Mérieux, Marcy I'Etoile, France) according to the supplier's instructions. Plasma concentrations of apolipoproteins AII, AI and CIII were determined as previously described (Raspe, Madsen et al. 1999) and (Asset, Staels et al. 1999).

To separate the lipoproteins according to size, 300 μl of plasma were loaded on a Sepharose 6HR 10/30 column (Pharmacia, Uppsala, Sweden) and eluted at a constant flow rate (0.2 ml/minute) in PBS (pH 7.2). Optical density of the eluent was recorded at 280 nm. 0.3 ml fractions were collected. Lipid concentrations in the different fractions were determined by a colorimetric assay (Bio-Mérieux, Marcy I'Etoile, France) according to the supplier's instructions.

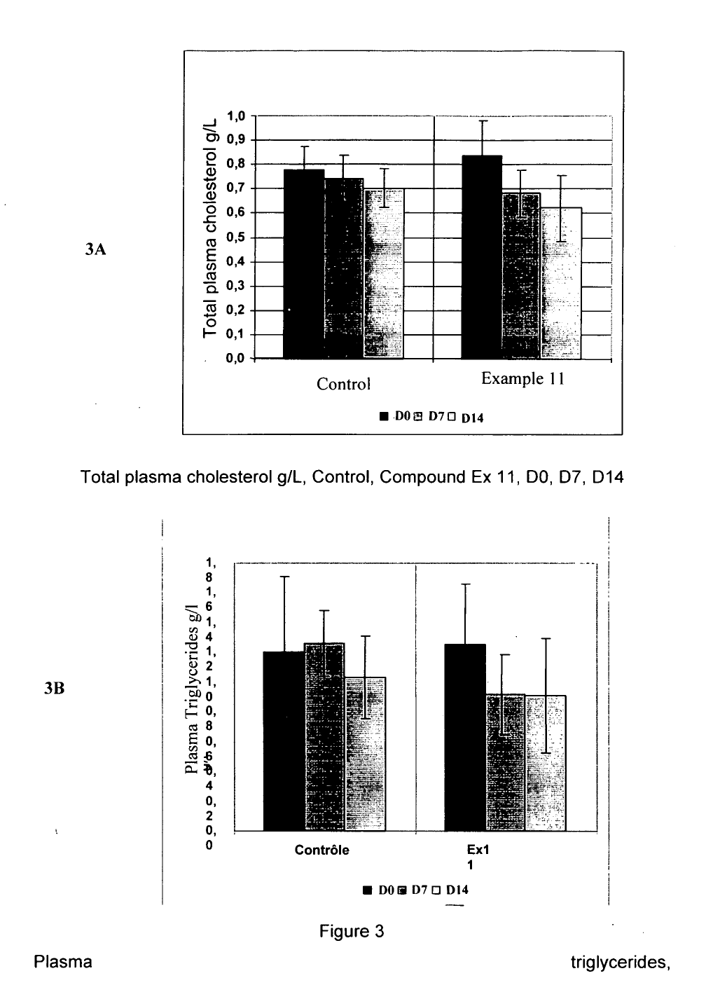

The results are presented in FIGS. 3A and 3B.

FIG. 3A shows the effects of treating Sprague-Dawley rats with the inventive compound of example 11 (300 mg/kg/day) on total plasma cholesterol. FIG. 3A shows that total plasma cholesterol levels were lowered by treating the animals with the inventive compound of example 11.

FIG. 3B shows the effects of treating Sprague-Dawley rats with the inventive compound of example 11 (300 mg/kg/day) on plasma triglycerides. FIG. 3B shows that plasma triglyceride levels were lowered by treating the animals with the inventive compound of example 11.

To separate the lipoproteins according to size, 300 μl of plasma were loaded on a Sepharose 6HR 10/30 column (Pharmacia, Uppsala, Sweden) and eluted at a constant flow rate (0.2 ml/minute) in PBS (pH 7.2). Optical density of the eluent was recorded at 280 nm. 0.3 ml fractions were collected. Lipid concentrations in the different fractions were determined by a calorimetric assay (Bio-Mérieux, Marcy I'Etoile, France) according to the supplier's instructions.

The results obtained confirm the in vivo effects of the inventive compounds. Moreover, a decrease in cholesterol in the different lipoparticle classes was seen, particularly in large particles (VLDL) and small particles (HDL). In addition, a typical distribution of triglycerides primarily into large lipoparticles was observed, and the triglyceride concentrations therein were markedly decreased. This decrease is characteristic of the effects of PPARα activators.

3) RNA Analysis

Total RNA was isolated from the liver fragments by extraction with a mixture of guanidine thiocyanate/phenol acid/chloroform as previously described (Raspé, Madsen et al., 1999). Messenger RNA was quantified by semi-quantitative or quantitative RT-PCR with the Light Cycler Fast Start DNA Master Sybr Green I kit (Hoffman-La Roche, Basel, Switzerland) on a Light Cycler System (Hoffman-La Roche, Basel, Switzerland). Primer pairs specific for the ACO, Apo CIII, Apo AI, CPT-I and CPT-II genes were used as probes. Primer pairs specific for the 36B4, β-actin and GAPDH genes were used as control probes.

The inventors thereby demonstrate an increase in the expression of genes involved in lipid transport or catabolism, confirming the previous results (PPAR activation and reduction of plasma cholesterol and triglyceride concentrations).

### Example 27

### Evaluation of the Antioxidant Properties of the Inventive Compounds

A—Protection Against LDL Oxidation Induced by Copper:

Oxidation of LDL is an important modification which plays a major role in the onset and development of atherosclerosis (Jurgens, Hoff et al. 1987). The following protocol allows demonstration of the antioxidant properties of compounds. Unless otherwise indicated, all reagents were from Sigma (St Quentin, France).

LDL were prepared as described in Lebeau et al. (Lebeau, Furman et al. 2000). The solutions of the test compounds were prepared at 10⁻² M in ethanol and diluted in PBS so that the final concentration ranged from 0.1 to 100 μM with a total ethanol concentration of 1% (VN).

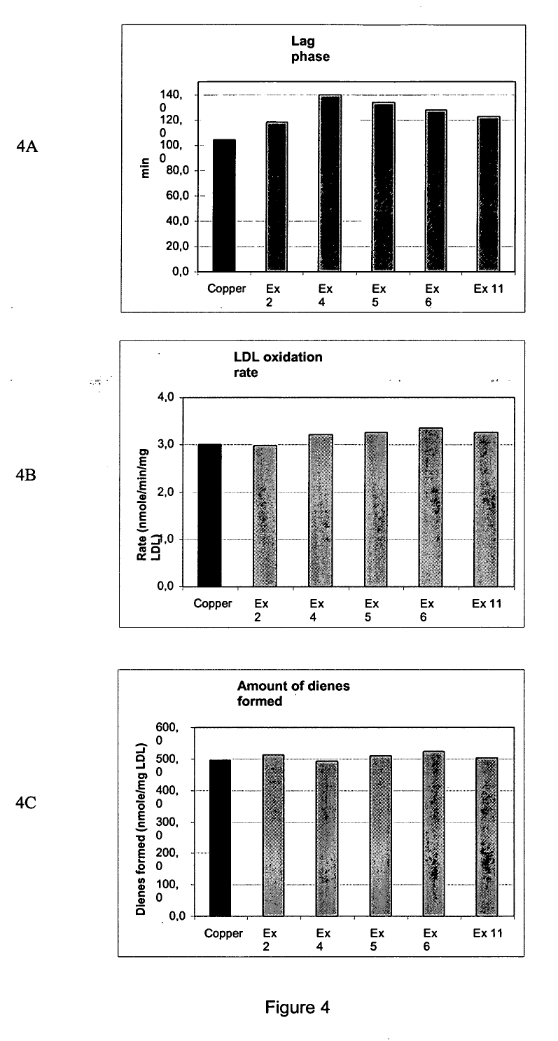

Before oxidation, EDTA was removed from the LDL preparation by dialysis. The oxidation reaction was then carried out at 30° C. by adding 100 μl of 16.6 μM CuSO₄ to 800 μl of LDL (125 μg protein/ml) and 100 μl of a test compound solution. The formation of dienes, the species to be followed, was measured by the optical density at 234 nm in the samples treated with the compounds in the presence or absence of copper. Optical density at 234 nm was measured every 10 minutes for 8 hours on a thermostated spectrophotometer (Kontron Uvikon 930). The analyses were carried out in triplicate. A compound was considered to have antioxidant activity when it shifted the lag phase latency relative to the control sample. The inventors demonstrate that the inventive compounds delayed LDL oxidation (induced by copper), indicating that the inventive compounds possess intrinsic antioxidant activity. FIG. 4 presents an example of the results obtained with the inventive compounds.

FIG. 4 shows that inventive compounds Ex 2, 4, 5, 6 and 11 exhibit intrinsic antioxidant properties and also promote a slowing of the rate of LDL oxidation induced by copper. In fact, the inventive compounds induced a shift in the lag phase latency, which was delayed by 13.4% when cells were treated with compound Ex 2 and up to 34.3% for compound Ex 4 (FIG. 4A). The inventive compounds did not appear to modify the oxidation rate (FIG. 4b) or the amount of dienes formed (FIG. 4C).

B—Evaluation of the Protection Conferred by the Inventive Compounds Against Lipid Peroxidation:

LDL oxidation was measured by the TBARS method (Thiobarbituric Acid Reactive Substances).

According to the same principle as that described hereinabove, LDL were oxidized in the presence of CuSO₄ and lipid peroxidation was evaluated as follows:

TBARS were measured by a spectrophotometric method, lipid hydroperoxidation was measured by using lipid peroxide-dependent oxidation of iodide to iodine. The results are expressed as nmol of malondialdehyde (MDA) or as nmol hydroperoxide/mg protein.

The results obtained by measuring the inhibition of conjugated diene formation, were confirmed by the experiments measuring LDL lipid peroxidation. Thus, the inventive compounds also afforded efficient protection of LDL against lipid peroxidation induced by copper (an oxidizing agent).

### Example 28

### Measurement of the Antioxidant Properties of the Inventive Compounds on Cell Cultures

A—Culture Protocol:

Neuronal, neuroblastoma (human) and PC12 cells (rat) were the cell lines used for this type of study. PC12 cells were prepared from a rat pheochromocytoma and have been characterized by Greene and Tischler (Greene and Tischler, 1976). These cells are commonly used in studies of neuron differentiation, signal transduction and neuronal death. PC12 cells were grown as previously described (Farinelli, Park et al. 1996) in complete RPMI medium (Invitrogen) supplemented with 10% horse serum and 5 % fetal calf serum.

Primary cultures of endothelial and smooth muscle cells were also used. Cells were obtained from Promocell (Promocell GmBH, Heidelberg) and cultured according to the supplier's instructions.

The cells were treated for 24 hours with different doses of the inventive compounds ranging from 5 to 100 μM. The cells were then recovered and the increase in expression of the target genes was evaluated by quantitative PCR.

B—mRNA Measurement:

mRNA was extracted from the cultured cells treated or not with the inventive compounds. Extraction was carried out with the reagents of the Absolutely RNA RT-PCR miniprep kit (Stratagene, France) as directed by the supplier. mRNA was then assayed by spectrometry and quantified by quantitative RT-PCR with a Light Cycler Fast Start DNA Master Sybr Green I kit (Roche) on a Light Cycler System (Roche, France). Primer pairs specific for the genes encoding the antioxidant enzymes superoxide dismutase (SOD), catalase and glutathione peroxidase (GPx) were used as probes. Primer pairs specific for the β-actin and cyclophilin genes were used as control probes.

An increase in mRNA expression of the antioxidant enzyme genes, measured by quantitative RT-PCR, was demonstrated in the different cell types used, when the cells were treated with the inventive compounds.

C—Control of Oxidative Stress:

Measurement of Oxidizing Species in the Cultured Cells:

The antioxidant properties of the compounds were also evaluated by means of a fluorescent tag the oxidation of which is followed by appearance of a fluorescence signal. The reduction in the intensity of the emitted fluorescence signal was determined in cells treated with the compounds in the following manner: PC12 cells cultured as described earlier (black 96-well plates, transparent bottom, Falcon) were incubated with increasing doses of hydrogen peroxide (0.25 mM-1 mM) in serum-free medium for 2 and 24 hours. After incubation, the medium was removed and the cells were incubated with 10 μM dichlorodihydrofluorescein diacetate solution (DCFDA, Molecular Probes, Eugene, USA) in PBS for 30 min at 37° C. in a 5 % CO₂ atmosphere. The cells were then rinsed with PBS. The fluorescence emitted by the oxidation tag was measured on a fluorimeter (Tecan Ultra 384) at an excitation wavelength of 495 nm and an emission wavelength of 535 nm. The results are expressed as the percentage of protection relative to the oxidized control.

Fluorescence intensity was lower in the cells incubated with the inventive compounds than in untreated cells. These findings indicate that the inventive compounds promote inhibition of the production of oxidative species in cells subjected to oxidative stress. The previously described antioxidant properties are also effective at inducing antiradical protection in cultured cells.

D—Measurement of Lipid Peroxidation:

The different cell lines (cell models noted hereinabove) and the primary cell cultures were treated as described earlier. The cell supernatant was recovered after treatment and the cells were lysed and recovered for determination of protein concentration. Lipid peroxidation was detected as follows: lipid peroxidation was measured by using thiobarbituric acid (TBA) which reacts with lipid peroxidation of aldehydes such as malondialdehyde (MDA). After treatment, the cell supernatant was collected (900 μl) and 90 μl of butylated hydroxytoluene were added (Morliere, Moysan et al. 1991). One milliliter of 0.375% TBA solution in 0.25 M hydrochloric acid containing 15% trichloroacetic acid was also added to the reaction medium. The mixture was heated at 80° C. for 15 min, cooled on ice and the organic phase was extracted with butanol. The organic phase was analyzed by spectrofluorimetry (λexc=515 nm and λem=550 nm) on a Shimazu 1501 spectrofluorimeter (Shimadzu Corporation, Kyoto, Japan). TBARS are expressed as MDA equivalents using tetra-ethoxypropane as standard. The results were normalized for protein concentration.

The decrease in lipid peroxidation observed in the cells treated with the inventive compounds confirms the previous results.

The inventive compounds advantageously exhibit intrinsic antioxidant properties allowing to slow and/or inhibit the effects of an oxidative stress. The inventors also show that the inventive compounds are capable of inducing the expression of genes encoding antioxidant enzymes. These particular features of the inventive compounds allow cells to more effectively fight against oxidative stress and therefore be protected against free radical-induced damage.

### Example 29

### Evaluation of Effects on the Expression of Enzymes Involved in Mitochondrial and Peroxisomal β-Oxidation

The inventive compounds which were tested are the compounds whose preparation is described in examples 2 to 23 hereinabove.

Fatty acids are an essential reservoir of energy. Mitochondrial and peroxisomal β-oxidation of fatty acids are the main catabolic pathways whereby this energy is mobilized. These two processes therefore play a key role in controlling serum levels of free fatty acids and in regulating triglyceride synthesis. The rate-limiting enzyme for peroxisomal B-oxidation is ACO. Mitochondrial β-oxidation is limited by the transport of fatty acids into the mitochondria, which depends on the activity of the enzymes CPT-I and CPT-II. Regulation of the expression of enzymes ACO, CPT-I and CPT-II is a crucial step in controlling peroxisomal and mitochondrial α-oxidation, respectively.

The inventive compounds induce the expression of ACO, CPT-I and CPT-II. Said activity was demonstrated in the following manner:

Rat hepatocytes were isolated by perfusing the livers of male Wistar OFA rats (Charles River, L'Arbresle, France) weighing between 175 and 225 g with a mixture of collagenase and thermolysin (Blendzyme 3, Roche, Basel, Switzerland). Rats were anesthetized with pentobarbital and the liver was perfused via the portal vein, first with 100 ml of wash buffer (Liver perfusion medium, Gibco, Paisley, UK) followed by 200 ml of the following digestion medium: HBSS depleted of calcium chloride and magnesium sulfate (Sigma, St Louis, Mich., USA) supplemented with 10 mM Hepes, pH 7.6, 4 mM calcium chloride and 7 mg of Blendzyme 3 according to a modification of the protocol described by (Raspe, Madsen et al. 1999). When cell viability as determined by the Trypan blue test (Sigma, St Louis, Mich., USA) exceeded 80%, the hepatocytes were spread on 6-well culture dishes at 10⁵ cells/cm² for quantification of messenger RNA. The cells were seeded and incubated for 4 hours in Williams E culture medium supplemented with 100 U/ml penicillin (Gibco, Paisley, UK), 2 mM L-glutamine (Gibco, Paisley, UK), 2% (V/V) UltroSER SF (Biosepra, Cergy St-Christophe, France), 0.2% (mN) bovine serum albumin (Sigma, St Louis, Mo., USA), 1 μM dexamethasone (Sigma, St Louis, Mμ, USA) and 100 nM T3 (Sigma, St Louis, Mo., USA). The experiment was then continued in the same culture medium depleted of Ultroser. The test compounds were added at the indicated concentration directly in the culture medium.

Total RNA was isolated from the liver fragments by extraction with a mixture of guanidine thiocyanate/phenol acid/chloroform as previously described (Raspé, Madsen et al., 1999). Messenger RNA was quantified by semi-quantitative or quantitative RT-PCR with the Light Cycler Fast Start DNA Master Sybr Green I kit (Hoffman-La Roche, Basel, Switzerland) on a Light Cycler System (Hoffman-La Roche, Basel, Switzerland). Primer pairs specific for the ACO, Apo CIII, Apo AI, CPT-I and CPT-II genes were used as probes. Primer pairs specific for the 36B4, β-actin and GAPDH genes were used as control probes (see table 1). RNA isolated from hepatocytes in primary culture described hereinabove or from liver fragments harvested from rats treated with the test compounds was quantified by semi-quantitative or quantitative RT-PCR as described in examples 25 and 26 with the help of primer pairs specific for the ACO, CPT-I and CPT-II genes.

### Example 30

### Evaluation of Fatty Acid Oxidation Capacities

The inventive compounds which were tested are the compounds whose preparation is described in examples 2 to 23 hereinabove.

The oxidation capacities of fatty acids determine serum levels of free fatty acids as well as the potential for triglyceride synthesis. Accumulation of free fatty acids in blood or of triglycerides outside of adipose tissue predisposes to insulin resistance. Furthermore, elevated plasma triglyceride levels are now thought to be a risk factor for cardiovascular diseases. An increase in fatty acid oxidation capacities is therefore of therapeutic interest.

The inventive compounds activate fatty acid oxidation by mitochondria and peroxisomes. Said ability was demonstrated as follows:

Mitochondrial CPT-I and CPT-II activity was tested according to the method described by Madsen et al. (Madsen et al., 1999).

ACO activity was measured as in Asiedu et al. (Asideu et al., 1995).

Mitochondrial and peroxisomal β-oxidation of fatty acids was evaluated as described by Hovik et al. (Hovik et al, 1990).

### Example 31

### Evaluation of the Effects on Reverse Cholesterol Transport

The inventive compounds which were tested are the compounds whose preparation is described in examples 2 to 23 hereinabove.

The negative correlation between HDL-cholesterol levels and cardiovascular diseases is now well established. The ability of a compound to increase reverse cholesterol transport (RCT) is considered a mechanism whereby HDL protect against atherosclerosis.

RCT is a process which allows excess cholesterol present in extrahepatic tissues to be recovered and exported to the liver where it undergoes transformation to bile acids which are then excreted in the bile.

The presence of macrophage-derived foam cells characterizes the first steps in the formation of atherosclerotic lesions.

Cholesterol outflow from macrophages is therefore a critical phase for preventing the formation of foam cells and, consequently, acts protectively against the development of atherosclerosis. The critical step of RCT is the transfer of excess cholesterol and cell membrane phospholipids to naiscent HDL. In this respect, the ABCA1 (ATP binding cassette A1) transporter plays a key role in this process and the expression thereof is correlated with a reduction in atherosclerotic plaque development through stimulation of cholesterol outflow from macrophages.

It was also recently shown that ABCA1 is a target gene of the LXRα nuclear receptor, itself a target gene of the PPARα and PPARγ receptors.

The inventive compounds induce the expression of LXRα and ABCA1 and stimulate cholesterol outflow in two in vitro models of primary and THP1 macrophages.

1—Measurement of ABCA1 and LXRα Expression:

a—Differentiation and treatment of primary and THP-1 human macrophages

THP-1 monocytes (ATCC, Rockville, Md., USA) were placed in 6-well culture dishes in the presence of PMA (phorbol myristate acetate) and fetal calf serum and incubated at 37° C. for 6 days to allow them to differentiate to macrophages. To obtain primary macrophages, mononuclear cells were isolated from human blood as previously described (Chinetti et al., 2001), placed in 6-well culture dishes and grown for 10 days in the presence of human serum to enable adherence and differentiation of the primary monocytes to macrophages.

Treatment with the different compounds was carried out for 48 hours in medium without human or fetal calf serum but supplemented with 1% Nutridoma HU serum (Boehringer).

b—Messenger RNA quantification

Total RNA was extracted from treated macrophages with the mini RNeasy kit (Qiagen, Hilden, Germany) according to the supplier's instructions, assayed by spectrometry and quantified by quantitative RT-PCR with the Light Cycler Fast DNA Master Green I kit (Hoffman-La Riche, Basel, Switzerland) on a Light Cycler System (Hoffman-La Riche, Basel, Switzerland). Primer pairs specific for the ABCA1 and LXRα genes were used as probes.

2—Measurement of Cholesterol Outflow:

a—Differentiation and treatment of primary and human THP-1 macrophages Macrophages were differentiated from THP-1 or primary monocytes as in the previous experiment (1—measurement of ABCA1 and LXRα expression).

b—Macrophages were pretreated for 24 hours with the compounds, but also every 24 hours throughout the duration of the experiment. Cholesterol loading was accomplished by incubation for 48 hours in the presence of acetylated LDL (50 μg/ml containing tritium-labelled cholesterol) in RPMI 1640 medium supplemented with 1% Nutridoma HU (Boehringer).

After this step, cells were washed twice with PBS and incubated for 24 hours in RPMI medium without Nutridoma, with or without apolipoprotein A-1. On completion of this step, the medium was recovered and intracellular lipids were extracted with a mixture of hexane/isopropanol, then dried under nitrogen. Outflow was quantified on a Tri-Carb® 2100 TR scintillation counter (Packard, Meriden, Conn., USA) by dividing the number of disintegrations counted in the medium by the total number of disintegrations counted in the medium and in the cells.

### Example 32

### Evaluation of the Effects on Metabolic Syndrome (Syndrome X) and Diabetes

The inventive compounds which were tested are the compounds whose preparation is described in examples 2 to 23 hereinabove.

Insulin resistance is the underlying basis of metabolic syndrome, which is characterized by glucose intolerance, hyperinsulinemia, dyslipidemia and hypertension. The combination of several cardiovascular risk factors which leads to an increased risk of cardiovascular disease secondary to atherosclerosis is responsible for most of the morbidity and mortality associated with type 2 diabetes. Pharmacological treatments of metabolic syndrome are therefore targeted chiefly at insulin resistance.

The inventive compounds attenuate the manifestations of metabolic syndrome (syndrome X), such as elevation of free fatty acids, hyperinsulinemia, hyperglycemia and the insulinemic response to glucose (glucose tolerance test), and of diabetes in two animal models of insulin resistance linked to metabolic syndrome: C57BL/6 mice maintained on a high fat diet, and obese Zucker rats (fa/fa). These properties were demonstrated as follows:

1) Treatment of Animals

Male C57BL/6 mice (Charles River, L'Arbresle, France) aged 6 weeks at the start of the experiment were randomly divided into groups of 6 animals such that body weight distribution was uniform. Mice were given a low-fat diet (UAR AO4), a high-fat diet (29% (m/m) coconut oil) or the same enriched diet supplemented with the test compounds. Obese (fa/fa) or non obese (fa/+) male Zucker rats aged 5 or 21 weeks (Charles River, L'Arbresle, France) were divided into groups of 8 animals selected such that the distribution of plasma cholesterol and triglyceride levels was uniform, and maintained on a standard diet. Animals were housed in a 12 hour light/dark cycle at a constant temperature of 20° C.±3° C. Animals had access to food and water ad libitum. Food intake and weight increase were recorded. The test compounds were suspended in a vehicle (0.5% carboxymethylcellulose (CMC) and 0.1% Tween) and administered by intragastric gavage at the indicated doses, once a day for 15 days. At the end of treatment, some animals underwent a glucose tolerance test as described hereinbelow. At the end of the experiment the other animals were weighed and sacrificed under anesthesia after a 5 hour fast. Blood was collected on EDTA. Plasma was prepared by centrifugation at 3000 rpm for 20 minutes. Liver samples were removed and stored frozen in liquid nitrogen for subsequent analysis.

2) Assay of Free Fatty Acids and Lipids

Free fatty acid levels vary in diabetic rats. Free fatty acid concentrations in serum or plasma were determined by a calorimetric enzymatic reaction “NEFA/FFA” WAKO (Labo Immuno Systems, Neuss, Germany) on serum or plasma.

Plasma lipid concentrations (total cholesterol and triglycerides) were determined by a calorimetric assay (Bio-Mérieux, Marcy I'Etoile, France) according to the supplier's instructions.

3) Glycemia Assay

Blood glucose was determined by a calorimetric enzymatic assay (Sigma Aldrich, St Louis, Mo., USA).

4) Insulin Assay

To demonstrate hyperinsulinemia, insulin levels were assayed with a radioassay kit (Mercodia, Uppsala, Sweden). Insulinemia was assayed on serum or plasma collected on EDTA.

5) Glucose Tolerance Test

Animals were anaesthetized after an 8 hour fast by intraperitoneal injection of pentobarbital sodium (50 mg/kg). To initiate the glucose tolerance test, glucose (1 g/kg) was injected into the peritoneal cavity before collecting blood samples from the caudal vein into heparinized tubes at 0, 15, 30, 45, and 60 minutes after the glucose load. Samples were stored on ice, the plasma was isolated and stored at −20° C. pending analysis.

### Example 33

### Evaluation of the Effects on Obesity

The inventive compounds which were tested are the compounds whose preparation is described in examples 2 to 23 hereinabove.

Obesity is accompanied by an increase in insulin resistance, type 2 diabetes and an increased risk of cardiovascular disease and cancer. It therefore plays a central role in some of the pathologies prevalent in the industrialized world and, for this reason, poses a major pharmacological challenge.

The inventive compounds reduce weight gain in two animal models of obesity: C57BL/6 mice fed a high-fat diet, and obese Zucker rats (fa/fa). These properties were demonstrated as follows:

1) Treatment of Animals

Male C57BL/6 mice (Charles River, L'Arbresle, France) aged 6 weeks at the start of the experiment were randomly divided into groups of 6 animals such that body weight distribution was uniform. Mice were given a low-fat diet (UAR AO4), a high-fat diet (29% (m/m) coconut oil) or the same enriched diet supplemented with the test compounds. Obese male Zucker rats (fa/fa) aged 5 weeks (Charles River, L'Arbresle, France) were divided into groups of 8 animals selected such that the distribution of plasma cholesterol and triglyceride levels was uniform, and maintained on a standard diet supplemented with the test compounds for 15 days. Animals were housed in a 12 hour light/dark cycle at a constant temperature of 20° C.±3° C. Animals had access to food and water ad libitum. Food intake and weight increase were recorded. At the end of the experiment the animals were weighed and sacrificed under anaesthesia. Plasma was prepared by centrifugation at 3000 rpm for 20 minutes. Liver and adipose tissue samples were removed, weighed and stored frozen in liquid nitrogen for subsequent analysis.

2) Assay of Leptin

Leptin, an obesity marker, was measured by the “Rat Leptin assay” kit from Linco Research (St Charles, Mich., USA).

### Example 34

### Evaluation of the Effects on Cell Growth

The inventive compounds which were tested are the compounds whose preparation is described in examples 2 to 23 hereinabove.

The inventive compounds decrease the growth of tumor cells.

This activity was observed by using the protocol described by Hvattum et al. (Hvattum et al. 1993).

### Example 35

### Evaluation of the Effects of the Compounds on Restenosis

The inventive compounds which were tested are the compounds whose preparation is described in examples 2 to 23 hereinabove.

Proliferation of smooth muscle cell is one of the principal components of atherogenesis, restenosis and hypertension associated with cardiovascular disease. The identification of inhibitors of said proliferation is therefore a worthwhile challenge in pharmacology.

The inventive compounds decrease the growth of vascular smooth muscle cells in vitro and reduce restenosis in vivo in a rat balloon angioplasty model. These properties were demonstrated as follows:

1) Measurement of Smooth Muscle Cell Proliferation.

Smooth muscle cells from the coronary artery or aorta were from Promocell (Heidelberg, Germany) and were grown according to the supplier's instructions in a special smooth muscle cell culture medium supplemented with 10% fetal calf serum. Cells grown to 50% confluence were made quiescent by omitting the serum for 24 hours. Cells were then treated for 3 to 6 days in the presence of mitogens (10% serum, 20 ng/ml βFGF or 2 U/ml a-thrombin) and the inventive compounds. At the end of the experiment, cells were trypsinized and counted on a hemocytometer.

2) Measurement of Restenosis in a Rat Balloon Coronary Angioplasty Model.

Adult Sprague-Dawley rats weighing 200 to 300 g (Iffa Credo, L'Arbresle, France) were housed in a 12 hour light/dark cycle at a constant temperature of 20° C.±3° C. After a 1-week acclimatization period, rats were weighed and divided into groups of 6 animals selected such that body weight distribution was uniform. The left internal coronary artery was damaged with a balloon as previously described (Ruef et al., 2000). The inventive compounds were suspended in a vehicle (0.5% carboxymethylcellulose (CMC) and 0.1% Tween) and administered by intragastric gavage at different doses, once a day for 4, 10 and 21 days. Treatment commenced one day before the balloon intervention. Animals had access to food and water ad libitum. Animals were then sacrificed and the coronary arteries fixed and analyzed as previously described (Ruef et al. 2000).

### Example 36

### Evaluation of the Effects of the Compounds on Hypertension

The inventive compounds which were tested are the compounds whose preparation is described in examples 2 to 23 hereinabove.

Hypertension is a major risk factor for cardiovascular disease and represents an important pharmacological challenge.

The inventive compounds lower blood pressure in vivo when administered to spontaneously hypertensive rats (SHR rats) used as a model of hypertension. These properties were demonstrated in the following manner:

1) Treatment of Animals

Adult SHR rats weighing 200 to 300 g (Harlan France, Gannat, France) were housed in a 12-hour light/dark cycle at a constant temperature of 20° C.±3° C. After a 1-week acclimatization period, rats were weighed and divided into groups of 6 animals selected such that body weight distribution was uniform. The inventive compounds were suspended in a vehicle (0.5% carboxymethylcellulose (CMC) and 0.1% Tween) and administered by intragastric gavage at different doses, once a day for 7 days. Animals had access to food and water ad libitum.

2) Blood Pressure Measurement

Blood pressure was measured as previously described (Siragy and Carey 1997).

### Example 37

### Evaluation of Antioxidant Properties on Cell Cultures

The inventive compounds which were tested are the compounds whose preparation is described in examples 2 to 23 hereinabove.

a) Procurement and Culture of Normal Human Keratinocytes

Normal human keratinocytes (NHK) were cultured from skin samples. The sample was first washed four times in PBS (Phosphate Buffered Saline—Invitrogen, France), then decontaminated by immersion for 30 seconds in two successive baths of 70% ethanol. Strips 3 mm wide were then cut, taking care to remove as much adipose tissue and dermis as possible. The strips were then incubated in a 0.25% trypsin solution (Invitrogen, France) at 37° C. for 4 hours.

After separation of epidermis from dermis, the epidermal preparation was filtered and centrifuged at 1000 rpm for 5 minutes. The pellet was taken up in KHN-D medium (DMEM+10% fetal calf serum (FCS)+hydrocortisone 0.4 μg/ml+EGF 10 ng/ml+10⁻⁹ M cholera toxin (Sigma, St Quentin, France)). Cells were counted, then seeded at 10×10⁶ cells/75 cm².

After 24 hours of culture, the medium was changed, cells were washed in PBS and K-SFM culture growth medium (Invitrogen, France) was then subsequently used. Cells were seeded at the desired density. Cells were grown in a 5 % CO₂ atmosphere at 37° C. and the culture medium was changed every 48 hours. Treatment with or without the inventive compounds took place before the cells reached confluence (70-80%), at which time the compounds were added directly to the culture medium at concentrations ranging from 1 to 100 μM.

b) Procurement and Culture of Human Fibroblasts

Normal human fibroblasts were cultured from skin samples. The samples were first washed 4 times in PBS (Phosphate Buffered Saline—Invitrogen, France), then decontaminated by immersion for 30 seconds in two successive baths of 70% ethanol. Pieces of dermis having an area of about 5 mm² were placed on the bottom of a Petri dish. Once the pieces adhered to the support (approximately 5 minutes), they were covered with 4 ml of DMEM medium supplemented with 20% FCS. The medium was replaced every two days. Cells migrated from the explant after one week and colonized the Petri dish. Once the cells had colonized the support, they were trypsinized, reseeded and cultured in DMEM+10% FCS (Invitrogen, France) at 37° C. in a 5% CO₂ atmosphere. Cells were treated when they reached confluence, the inventive compounds being added directly to the culture medium at concentrations ranging from 1 to 100 μM.

c) Messenger RNA Quantification

mRNA was extracted from the normal human keratinocyte and fibroblast cultures treated or not with the inventive compounds. Extraction was carried out with the reagents in the Absolutely RNA RT-PCR miniprep kit (Stratagene, France) according to the supplier's instructions. mRNA was then assayed by spectrometry and quantified by quantitative RT-PCR using the Light Cycler Fast Start DNA Master Sybr Green I kit (Roche, France) on a Light Cycler System (Roche, France). Primer pairs specific for the genes encoding superoxide dismutase (SOD) and glutathione peroxidase (GPx), two antioxidant enzymes, were used as probes. Primer pairs specific for the 36B4, β-actin and GAPDH genes were used as controls (see Table I).

d) Determination of Glutathione Peroxidase (GPx) Activity

Glutathione peroxidase activity was measured on protein extracts of cells (keratinocytes, fibroblasts) treated or not with the inventive compounds at concentrations ranging from 1 to 100 μM. GPx activity was also determined under conditions of cellular stress (0.5 mM paraquat or 0.6 mM H₂O₂, which induce the formation of reactive oxygen species). Activity in the protein extracts was measured with the Glutathione Peroxidase Cellular Activity Assay Kit (Sigma) according to the supplier's instructions. Indirect determination is based on oxidation of glutathione to oxidized glutathione catalyzed by glutathione peroxidase. Reconversion to the non-oxidized form is catalyzed by glutathione reductase and NADPH (β-nicotinamide adenine dinucleotide phosphate). The decrease in NADPH absorbance was measured at 340 nm on a Shimazu 1501 spectrofluorimeter (Shimadzu Corporation, Kyoto, Japan) and reflects GPx activity, since GPx is the limiting factor in this reaction.

e) Determination of Lipid Peroxidation

Reagents were from Sigma (St Quentin, France) unless otherwise indicated.

Lipid peroxidation was measured by assaying malondialdehyde (MDA) using thiobarbituric acid (TBA). After the treatments, the cell supernatant was collected (900 μl) and 90 μl of butylated hydroxytoluene were added (Morliere P. et al., 1991). One milliliter of a 0.375% solution of TBA in 0.25 M HCl containing 15% trichloroacetic acid was also added to the supernatant. The mixture was heated at 80° C. for 15 minutes, cooled on ice and the organic phase was extracted with butanol. The organic phase was analyzed by spectrofluorimetry (λex=515 nm and λem=550 nm), on a Shimazu 1501 spectrofluorimeter (Shimadzu Corporation, Kyoto, Japan). TBARS were expressed as MDA equivalents using tetra-ethoxypropane as standard. The results were normalized against the protein content of the cells. Lipid peroxidation was induced by treating the cells with 0.5 mM paraquat (inducer of reactive oxygen species) or 0.6 mM hydrogen peroxide for 4 hours. The anti-radical protection provided by the inventive compounds at concentrations of 1 to 100 μM was evaluated by a 24-hour pretreatment, before induction of lipid peroxidation.

Treatment of the cells (keratinocytes and fibroblasts) with the inventive compounds promoted an increase in the expression of mRNA encoding the antioxidant enzymes SOD and GPx. This increase in transcriptional activity was also manifested as an increase in the activity of said enzymes. Incubation of the cells with the inventive compounds reduces lipid peroxidation induced by an oxidizing agent.

In this manner the antioxidant properties of the inventive compounds on cell cultures were demonstrated.

### Example 38

### Evaluation of Anti-Inflammatory Properties on Reconstructed Epidermis

Reconstructed epidermis was supplied by SkinEthic (Nice, France). Epidermis was used at day 17 (0.63 cm²) when the horny layer was present and the epithelial ultrastructure resembled that of human epidermis in vivo. Reconstructed epidermis was maintained in culture as instructed by the supplier. The reconstructed epidermis was treated with the inventive compounds at doses ranging from 2 to 10 mg/cm² for 24 and 72 hours.

The inventive compounds which were tested are the compounds whose preparation is described in examples 2 to 23 hereinabove.

a) Measurement of Anti-Inflammatory Properties

The reconstructed epidermis was preincubated with the inventive compounds at concentrations ranging from 2 to 10 mg/cm² for 24 hours, then treated with 0.4% SDS or 1 μg of TPA (12-O-tetradecanoylphorbol-13-acetate) for 6 hours. The anti-inflammatory potential of the compounds was evaluated by an ELISA method. The culture media (underneath) of the control or treated epidermis were collected and frozen at −20° C. Interleukin 1-α (IL1-α) was quantified with the ELISA IL1-α Kit (R&D System, UK) according to the supplier's instructions.

b) Messenger RNA Quantification

mRNA was extracted from the reconstructed epidermis treated or not with the inventive compounds as described hereinabove. Extraction was carried out with the reagents of the Absolutely RNA RT-PCR Miniprep Kit (Stratagene) according to the supplier's instructions and mRNA was then assayed by spectrometry and quantified by quantitative RT-PCR using the Light Cycler Fast Start DNA Master Sybr Green I kit (Roche) on a Light Cycler System (Roche). Primer pairs specific for the IL1 (interleukin 1) and IL6 genes were used as probes. Primer pairs specific for the 36B4, β-actin and GAPDH genes were used as control probes (see Table I).

Application of the inventive compounds on reconstructed epidermis led to a significant reduction in the secretion of the inflammatory cytokine, interleukin 1, after an inflammatory stress. This decrease in secretion was correlated with a decrease in expression of the mRNA encoding this cytokine, as measured by quantitative RT-PCR. These findings concerning treatment of reconstructed epidermis with the inventive compounds indicate that the latter have anti-inflammatory properties.

### Example 39

### Evaluation of Antioxidant Properties on Reconstructed Epidermis

The inventive compounds which were tested are the compounds whose preparation is described in examples 2 to 23 hereinabove.

Reconstructed epidermis was supplied by SkinEthic (Nice, France). Epidermis was used at day 17 (0.63 cm²) when the horny layer was present and the epithelial ultrastructure resembled that of human epidermis in vivo. Reconstructed epidermis was maintained in culture as instructed by the supplier. The reconstructed epidermis was treated with the inventive compounds at doses ranging from 2 to 10 mg/cm² for 24 and 72 hours.

a) Messenger RNA Quantification

mRNA was extracted from keratinocytes (from the reconstructed epidermis treated or not with the inventive compounds). Extraction was carried out with the reagents of the Absolutely RNA RT-PCR Miniprep Kit (Stratagene) according to the supplier's instructions and mRNA was then assayed by spectrometry and quantified by quantitative RT-PCR using the Light Cycler Fast Start DNA Master Sybr Green I kit (Roche) on a Light Cycler System (Roche). Primer pairs specific for the genes encoding superoxide dismutase (SOD) and glutathione peroxidase (GPx), two antioxidant enzymes, were used as probes. Primer pairs specific for the 36B4, β-actin and GAPDH genes were used as controls (see Table I).

b) Determination of Glutathione Peroxidase (GPx) Activity

Glutathione peroxidase activity was measured on protein extracts of reconstructed epidermis treated or not with the inventive compounds (2 to 10 mg/cm²). GPx activity was also determined under conditions of cellular stress (0.5 mM paraquat, an inducer of reactive oxygen species). Activity in the protein extracts was measured with the Glutathione Peroxidase Cellular Activity Assay Kit (Sigma) according to the supplier's instructions. Indirect determination is based on oxidation of glutathione to oxidized glutathione catalyzed by glutathione peroxidase. Reconversion to the non-oxidated form is catalyzed by glutathione reductase and NADPH (β-nicotinamide adenine dinucleotide phosphate). The decrease in NADPH absorbance was measured at 340 nm on a Shimazu 1501 spectrofluorimeter (Shimadzu Corporation, Kyoto, Japan) and reflects GPx activity, since GPx is the limiting factor in this reaction.

Quantitative RT-PCR analysis showed that the expression of antioxidant genes (SOD and GPx) was significantly increased. The increase in the amount of mRNA coding for GPx may be correlated with the increase in the activity of this antioxidant enzyme in reconstructed epidermis. These results highlight the antioxidant properties of the inventive compounds.

### Example 40

### Cosmetic Composition: Anti-Age Daytime Facial Cream

| | |
|---|---|
| Glyceryl stearate + PEG-100 stearate | 6.00% |
| Squalane | 3.00% |
| Hydrogenated polyisobutene | 3.00% |
| Glycerol tricaprylate/caprate | 3.00% |
| Glycerin | 2.00% |
| Octyl methoxycinnamate | 2.00% |
| Beeswax | 1.50% |
| Ketostearyl octanoate | 1.50% |
| Cetyl alcohol | 1.00% |
| Stearyl alcohol | 1.00% |
| Dimethicone | 1.00% |
| Xanthan gum | 0.20% |
| Carbomer | 0.15% |
| 1,3-ditetradecylthioacetylamino-2- | 0.10% |
| (tetradecylthioacetyloxy)propane | |
| Neutralizer | qs. |
| Preservatives | qs. |
| Fragrance, Coloring agents | qs. |
| Water | q.s. 100.00% |

### Example 41

### Cosmetic Composition: Anti-Wrinkle Facial Emulsion-Gel

| | |
|---|---|
| Glycerin | 5.00% |
| Caprylic/capric/Succinic triglycerides | 3.00% |
| Octyl methoxycinnamate | 1.00% |
| 3-tetradecylthioacetylamino-1,2- | 0.50% |
| (ditetradecylthioacetyloxy)propane | |
| Acrylates/C10-30 alkyl acrylate crosspolymer | 0.50% |
| Wheat protein hydrolysate | 0.50% |
| Dimethicone copolyol | 0.50% |
| Neutralizer | q.s. |
| Preservatives | q.s. |
| Fragrance, coloring agents | q.s. |
| Water | q.s. 100.00% |

### BIBLIOGRAPHIC REFERENCES

Adams, E. P., F. P. Doyle, et al. (1960). “Antituberculous sulphur compounds. Part IV. Some dimercaptopropyl esters and related dithiouronium bromides.” J Chem Soc: 2674-80.

Antoniadou-Vyzas, A., G. B. Foscolos, et al. (1986). “Di-adamantane derivatives of a,o-polymethylenediamines with antimicrobial activity.” Eur J Med Chem Chim Ther 21 (1): 73-74.

Asiedu, D. K., A. al-Shurbaji, et al. (1995). “Hepatic fatty acid metabolism as a determinant of plasma and-liver triacylglycerol levels. Studies on tetradecylthioacetic and tetradecylthiopropionic acids.” Eur J Biochem 227(3): 715-22.

Asset, G., B. Staels, et al. (1999). “Effects of Pinus pinaster and Pinus koraiensis seed oil supplementation on lipoprotein metabolism in the rat.” Lipids 34(1): 39-44.

Bhatia, S. K. and J. Hajdu (1987). “Stereospecific synthesis of 2-thiophosphatidylcholines; a new class of biologically active phospholipid analogues.” Tetrahedron Lett 28(33): 3767-3770.

Chinetti, G., S. Lestavel, et al. (2001). “PPAR-alpha and PPAR-gamma activators induce cholesterol removal from human macrophage foam cells through stimulation of the ABCA1 pathway.” Nat Med 7(1): 53-8.

Farinelli, S. E., D. S. Park, et al. (1996). “Nitric oxide delays the death of trophic factor-deprived PC12 cells and sympathetic neurons by a cGMP-mediated mechanism.” J Neurosci 16(7): 2325-34.

Gaffney, P. R. J. and C. B. Reese (1997). “Preparation of 2-O—arachidonoyl-1-O-stearoyl-sn-glycerol and other di-O-acyl glycerol derivatives.” Tetrahedron Lett 38(14): 2539-2542.

Greene, L. A. and A. S. Tischler (1976). “Establishment of a noradrenergic clonal line of rat adrenal pheochromocytoma cells which respond to nerve growth factor.” Proc Natl Acad Sci USA 73(7): 2424-8.

Gronowitz, S., B. Herslöf, et al. (1978). “Syntheses and chroptical properties of some derivatives of 1-thioglycerol.” Chem Phys Lipids 22: 307-320.

Hovik, R., H. Osmundsen, et al. (1990). “Effects of thia-substituted fatty acids on mitochondrial and peroxisomal beta-oxidation. Studies in vivo and in vitro.” Biochem J 270(1): 167-73.

Hvattum, E., H. J. Grav, et al. (1993). “Hormonal and substrate regulation of 3-thia fatty acid metabolism in Morris 7800 C1 hepatoma cells.” Biochem J 294(Pt 3): 917-21.

Jurgens, G., H. F. Hoff, et al. (1987). “Modification of human serum low density lipoprotein by oxidation—characterization and pathophysiological implications.” Chem Phys Lipids 45(2-4): 315-36.

Kitchin, J., R. C. Bethell, et al. (1994). “Synthesis and structure-activity relationships of a series of penicillin-derived HIV proteinase inhibitors: heterocyclic ring systems containing P1′ and P2′ substituents.” J Med Chem 37(22): 3707-16.

Kotsovolou, S., A. Chiou, et al. (2001). “Bis-2-oxo amide triacylglycerol analogues: a novel class of potent human gastric lipase inhibitors.” J Org Chem 66(3): 962-7.

Lebeau, J., C. Furman, et al. (2000). “Antioxidant properties of di-tert-butylhydroxylated flavonoids.” Free Radic Biol Med 29(9): 900-12.

Madsen, L., A. Garras, et al. (1999). “Mitochondrial 3-hydroxy-3-methylglutaryl coenzyme A synthase and carnitine palmitoyltransferase II as potential control sites for ketogenesis during mitochondrion and peroxisome proliferation.” Biochem Pharmacol 57(9): 1011-9.

Marx, M. H., C. Piantadosi, et al. (1988). “Synthesis and evaluation of neoplastic cell growth inhibition of 1-N-alkylamide analogues of glycero-3-phosphocholine.” J Med Chem 31(4): 858-63.

Morliere, P., A. Moysan, et al. (1991). “UVA-induced lipid peroxidation in cultured human fibroblasts.” Biochim Biophys Acta 1084(3): 261-8.

Morris, A. D., G. Atassi, et al. (1997). “The synthesis of novel melphalan derivatives as potential antineoplastic agents.” Eur J Med Chem 32(4): 343-50.

Murata, M., S. Ikoma, et al. (1991). “New synthesis of 2-thio-PAF and related compounds as substrates of PAF acetylhydrolase and phospholipase A2.” Chem Pharm Bull 39(5): 1335-1336.

Nazih, A., Y. Cordier, et al. (1999). “Synthesis and stability study of the new pentaammonio lipidpcTG90, a gene transfer agent.” Tetrahedron Lett 40(46): 8089-92.

Nazih, A., Y. Cordier, et al. (2000). “One-pot transformation of a t-butyl carbamate to a bromoacetamide in the synthesis of the gene transfer agent pcTG201.” Synlett 5: 635-6.

Rahman, M. D., D. L. Ziering, et al. (1988). “Effects of sulfur-containing analogues of stearic acid on growth and fatty acid biosynthesis in the protozoan Crithidia fasciculata.” J Med Chem 31(8): 1656-9.

Ramalingan, K., N. Raju, et al. (1995). “Synthesis of nitroimidazole substituted 3,3,9,9-tetramethyl-4,8-diaza-undecane-2,10-dione dioximes (propylene amine oximes, PnAOs): ligands for technetium-99m complexes with potential for imageing hypoxic tissue.” Tetrahedron 51(10): 2875-94.

Raspe, E., L. Madsen, et al. (1999). “Modulation of rat liver apolipoprotein gene expression and serum lipid levels by tetradecylthioacetic acid (TTA) via PPARalpha activation.” J Lipid Res 40(11): 2099-110.

Ruef, J., S. Q. Liu, et al. (2000). “Involvement of aldose reductase in vascular smooth muscle cell growth and lesion formation after arterial injury.” Arterioscler Thromb Vasc Biol 20(7): 1745-52.

Shealy, Y. F., J. L. Frye, et al. (1984). “Synthesis and properties of some 13—Cis- and all-trans-retinamides.” J Pharm Sci 73(6): 745-51.

Siragy, H. M. and R. M. Carey (1997). “The subtype 2 (AT2) angiotensin receptor mediates renal production of nitric oxide in conscious rats.” J Clin Invest 100(2): 264-9.

Spooner, P. J., S. B. Clark, et al. (1988). “The ionization and distribution behavior of oleic acid in chylomicrons and chylomicron-like emulsion particles and 30 the influence of serum albumin.” J Biol Chem 263(3): 1444-53.

Urakami, C. and K. Kakeda (1953). “Derivatives of dl-aminopropanediols.” Bull Chem Soc Jpn 26(5): 276-278.

1-18. (canceled)

19. A method for the treatment of a pathology involving a deregulation of lipid and/or glucose metabolism, a pathology related to inflammation, and/or a pathology related to cell proliferation and/or differentiation, by administering into a subject in need of such treatment an effective amount of at least one compound of the invention represented by general formula (I) 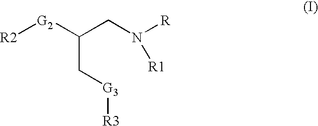
in which:
G2 and G3 independently represent an oxygen atom, a sulfur atom or a N—R4 group, G2 and G3 not simultaneously representing a N—R4 group,
R and R4 independently represent a hydrogen atom or a linear or branched alkyl group, saturated or not, optionally substituted, containing from 1 to 5 carbon atoms,
R1, R2 and R3, which are the same or different, represent a hydrogen atom, a CO—R5 group or a group corresponding to the formula CO—(CH2)2n+1-X—R6, at least one of the groups R1, R2 or R3 being a group corresponding to the formula CO—(CH2)2n+1-X—R6,
R5 is a linear or branched alkyl group, saturated or not, optionally substituted, possibly comprising a cyclic group, the main chain of which contains from 1 to 25 carbon atoms,
X is a sulfur atom, a selenium atom, a SO group or a SO2 group,
n is a whole number comprised between 0 and 11,
R6 is a linear or branched alkyl group, saturated or not, optionally substituted, possibly comprising a cyclic group, the main chain of which contains from 3 to 23 carbon atoms, preferably 10 to 23 carbon atoms and optionally one or more heterogroups, selected in the group consisting of an oxygen atom, a sulfur atom, a selenium atom, a SO group and SO2 group.,
the optical and geometrical isomers, racemates, salts, hydrates thereof and the mixtures thereof.

20. The method according to claim 19, wherein a single one of the groups R1, R2 or R3 represents a hydrogen atom.

21. The method according to claim 19, wherein, in the CO—(CH2)2n+1-X—R6 group, X represents a sulfur or selenium atom and advantageously a sulfur atom.

22. The method according to claim 19, wherein, in the CO—(CH2)2n+1-X—R6 group, n is comprised between 0 and 3, more specifically comprised between 0 and 2 and in particular is equal to 0.

23. The method according to claim 19, wherein R6 contains one or more heterogroups, preferably 0, 1 or 2, more preferably 0 or 1, selected in the group consisting of an oxygen atom, a sulfur atom, a selenium atom, a SO group and a SO2 group.

24. The method according to claim 19, wherein the group having the formula CO—(CH2)2n+1-X—R6 is the CO—CH2-S—C14H29 group.

25. The method according to claim 19, wherein at least one of the groups R1, R2 and R3 represents a CO—(CH2)2n+1-X—R6 group in which X represents a sulfur or selenium atom and preferably a sulfur atom and/or R6 is a saturated and linear alkyl group containing from 3 to 23 carbon atoms, preferably 13 to 20 carbon atoms, preferably 14 to 17, more preferably 14 to 16, and even more preferably 14 carbon atoms.

26. The method according to claim 19, wherein at least two of the groups R1, R2 and R3 are CO—(CH2)2n+1-X—R6 groups, which are the same or different, in which X represents a sulfur or selenium atom and preferably a sulfur atom.

27. The method according to claim 19, wherein G2 represents an oxygen or sulfur atom, and preferably an oxygen atom.

28. The method according to claim 19, wherein R2 represents a group corresponding to the formula CO—(CH2)2n+1-X—R6.

29. The method according to claim 19, wherein:
G3 is a N—R4 group in which R4 is a hydrogen atom or a methyl group, and G2 is an oxygen atom; and/or
R2 represents a CO—(CH2)2n+1-X—R6 group.

30. The method according to claim 19, wherein R1, R2 and R3, which are the same or different, preferably the same, represent a CO—(CH2)2n+1-X—R6 group, in which X represents a sulfur or selenium atom and preferably a sulfur atom and/or R6 is a saturated and linear alkyl group containing from 13 to 17 carbon atoms, preferably 14 to 17, even more preferably 14 carbon atoms, in which n is preferably comprised between 0 and 3, and in particular is equal to 0, more specifically, R1, R2 and R3, the same or different, representing CO—CH2-S—C14H29 groups.

31. The method according to claim 19, wherein the compound represented by formula (I) is selected in the group consisting of:
3-(tetradecylthioacetylamino)propane-1,2-diol;
1-tetradecylthioacetylamino-2,3-(dipalmitoyloxy)propane;
3-tetradecylthioacetylamino-1,2-(ditetradecylthioacetyloxy)propane;
3-palmitoylamino-1,2-(ditetradecylthioacetyloxy)propane;
1,3-di(tetradecylthioacetylamino)propan-2-ol;
1,3-diamino-2-(tetradecylthioacetyloxy)propane;
1,3-ditetradecylthioacetylamino-2-(tetradecylthioacetyloxy)propane;
1,3-dioleylamino-2-(tetradecylthioacetyloxy)propane;
1,3-ditetradecylthioacetylamino-2-(tetradecylthioacetylthio)propane; and
1-tetradecylthioacetylamino-2,3-di(tetradecylthioacetylthio)propane.

32. The method according to claim 19, wherein the pathology related to deregulations of lipid and/or glucose metabolism is selected in the group consisting of syndrome X, diabetes, atherosclerosis and obesity.

33. The method according to claim 19, wherein the pathology related to inflammation is selected in the group consisting of atherosclerosis, an allergy, asthma, eczema, psoriasis and pruritus.

34. The method according to claim 19, wherein the pathology related to cell proliferation and/or differentiation is selected in the group consisting of carcinogenesis, psoriasis and atherosclerosis.

35. The method according to claim 19, wherein the pathology is selected in the group consisting of cardiovascular diseases, syndrome X, restenosis, type I or II diabetes, preferably type II, obesity, hypertension, in particular arterial hypertension, cancers, in particular cancer of the anus, rectum, colon, intestine, duodenum, stomach, prostate, testicles, bladder, kidney, pancreas, liver, larynx, breast, lungs, leukemia and melanomas, and dermatological diseases.

36. The method according to claim 19, to prevent or treat the effects of intrinsic or extrinsic skin ageing.
